(19) United States
(12) Patent Application Publication
(10) Pub. No.: US 2022/0196642 A1
(43) Pub. Date: Jun. 23, 2022

(54) ENGINEERED OPIOID BIOSENSORS

(71) Applicants: California Institute of Technology, Pasadena, CA (US); Howard Hughes Medical Institute, Chevy Chase, MD (US)

(72) Inventors: Anand K. Muthusamy, Pasadena, CA (US); Henry A. Lester, Pasadena, CA (US); Loren Looger, Ashburn, VA (US); Jonathan S. Marvin, Ashburn, VA (US)

(21) Appl. No.: 17/553,725

(22) Filed: Dec. 16, 2021

Related U.S. Application Data
(60) Provisional application No. 63/126,991, filed on Dec. 17, 2020.
(60) Provisional application No. 63/287,008, filed on Dec. 7, 2021.

(51) Int. Cl. G01N 33/542; C07K 14/435; G01N 33/94

(52) U.S. Cl. CPC G01N 33/542; C07K 2319/20; G01N 33/9486; C07K 14/43595

(57) ABSTRACT

Disclosed herein include engineered opioid biosensors, and related compositions, vectors, cells, and systems. Also disclosed include methods that provide opioid biosensors with sensitivity and selectivity suitable for continuous opioid monitoring as well as the use of the opioid biosensors for detecting one or more specific opioids. The opioid biosensors, which are capable of undergoing a detectable conformational change upon binding to an opioid, can each comprise a first periplasmic binding protein (PBP) domain and a second PBP domain connected to the first PBP domain, wherein at least one of the first PBP domain and the second PBP domain comprises one or more mutations.

### RELATED APPLICATIONS

The present application claims priority under 35 U.S.C. § 119(e) to U.S. Provisional Application No. 63/126,991, filed Dec. 17, 2020 and U.S. Provisional Application No. 63/287,008, filed Dec. 7, 2021, the contents of which are herein expressly incorporated by reference in their entireties.

### STATEMENT REGARDING FEDERALLY SPONSORED R&D

This invention was made with government support under Grant No(s). GM123582 and DA049140 awarded by the National Institutes of Health. The government has certain rights in the invention.

### REFERENCE TO SEQUENCE LISTING

The present application is being filed along with a Sequence Listing in electronic format. The Sequence Listing is provided as a file entitled Sequence_Listing_30KJ_302436_US, created Dec. 16, 2021, which is 135 kilobytes in size. The information in the electronic format of the Sequence Listing is incorporated herein by reference in its entirety.

### BACKGROUND

### Field

The present disclosure relates generally to the field of protein engineering and the uses of the engineered proteins. Most particularly, the application relates to engineered biosensors, the methods, and compositions for expressing the engineered biosensors, and the uses of the engineered biosensors.

### Description of the Related Art

Opioid use poses significant risks to human health. Synthetic opioids in particular have driven an “opioid epidemic” of opioid use disorder (OUD) and overdose death over the past decade. OUD is marked by the development of tolerance that drives increasing drug consumption and the risk of uncontrolled consumption (Williams '13; Evans and Cahill '16). The rise of opioid use disorder and death by overdose necessitates new tools for both neuropharmacology and clinical diagnostic methods.

The pharmacokinetics of opioids can be defined at the whole-body level which provides the means to personalize opioid regimens and at the subcellular level which addresses cellular tolerance. The whole-body level pharmacokinetics is conventionally addressed with blood draw and LC-MS quantification, but this method is laborious and requires patients to visit a clinical setting. The subcellular level pharmacokinetic measurement requires an in-situ readout with subcellular resolution. Several minimally invasive technologies exist for continuous analyte monitoring for endogenous analytes, but no suitable opioid-binding moieties exist to provide continuous opioid monitoring. Physicians routinely employ “opioid rotations” cycling through multiple drugs to find the idea fit for an individual or to exploit incomplete cross-tolerance (Knotkova '09).

### SUMMARY

Disclosed herein include a polypeptide comprising a first mutated periplasmic binding protein (PBP) domain and a second PBP domain connected to the first PBP domain, wherein at least one of the first and second PBP domain comprises at least one amino acid substitution mutation selected from the positions functionally equivalent to K10, N11, Q15, T43, T68, T325, K330, D341, Y357, A360, E391, R395, V405, F436, H455, and D490 of the amino acid sequence of SEQ ID NO: 29. In some embodiments, the polypeptide is an isolated or substantially purified polypeptide. In some embodiments, the polypeptide is in solution or in a lyophilized form. Disclosed herein include a composition comprising any of the polypeptide disclosed herein, and one or more of excipients, surfactants, and salts,

Disclosed herein include opioid biosensors. In some embodiments, the opioid biosensor comprises a first mutated PBP domain and a second PBP domain connected to the first PBP domain, wherein the opioid biosensor is capable of undergoing a detectable conformational change upon binding to an opioid, wherein at least one of the first and second PBP domain comprises at least one amino acid substitution mutation selected from the positions functionally equivalent to K10, N11, Q15, T43, T68, T325, K330, D341, Y357, A360, E391, R395, V405, F436, H455, and D490 of the amino acid sequence of SEQ ID NO: 29.

The opioid can be, for example, an opioid specific to a kappa opioid receptor, delta opioid receptor, or mu opioid receptor. In some embodiments, the opioid is codeine, morphine, morphinan, fentanyl, phenyl piperazine, hydrocodone, oxycodone, oxymorphone, heroin, or benzenoid monoamine. In some embodiments, the first PBP domain comprises an amino acid sequence at least 80% identical to position 1-75 of a sequence selected from SEQ ID NOs: 1-27 and the second PBP domain comprises an amino acid sequence at least 80% identical to positions 325-521 of a sequence selected from SEQ ID Nos: 1-27. In some embodiments, the first PBP domain comprises an amino acid sequence at least 80% identical to position 1-75 of SEQ ID NO: 29 and the second PBP domain comprises an amino acid sequence at least 80% identical to positions 325-521 of SEQ ID NO: 29. In some embodiments, the first PBP domain comprises an amino acid sequence at least 90% identical to position 1-75 of a sequence selected from SEQ ID NOs: 1-27 and the second PBP domain comprises an amino acid sequence at least 90% identical to positions 325-521 of a sequence selected from SEQ ID Nos: 1-27. In some embodiments, the first PBP domain comprises an amino acid sequence at least 90% identical to position 1-75 of SEQ ID NO: 29 and the second PBP domain comprises an amino acid sequence at least 90% identical to positions 325-521 of SEQ ID NO: 29. In some embodiments, the first PBP domain comprises an amino acid sequence of position 1-75 of a sequence selected from SEQ ID NOs: 1-25 and the second PBP domain comprises an amino acid sequence of positions 325-521 of a sequence selected from SEQ ID Nos: 1-25.

In some embodiments, the at least one amino acid substitution mutation comprises a substitution mutation at F436. In some embodiments, the at least one amino acid substitution mutation comprises a substitution mutation at N11. In some embodiments, the at least one amino acid substitution mutation comprises a substitution mutation of N11V or a substitution mutation homologous to N11V. In some embodiments, the polypeptide or the opioid biosensor comprises a substitution mutation of N11V and a Phe at position 436 and the opioid biosensor binds specifically to S-methadone. In some embodiments, the polypeptide or the opioid biosensor comprises at least one additional substitution mutation selected from: K10I, Q15G, T43E, T68H, T325S, K330G, D341R, Y357G, A360T, E391F, R395G, V405L, H455A, D490G, and a combination thereof. In some embodiments, the polypeptide or the opioid biosensor binds specifically to S-methadone and the first PBP domain comprises an amino acid sequence at least 90% identical to position 1-75 of SEQ ID NO: 4 or SEQ ID NO: 25 and the second PBP domain comprises an amino acid sequence at least 90% identical to positions 325-521 of SEQ ID NO: 4 or SEQ ID NO: 25. In some embodiments, the polypeptide or the opioid biosensor binds specifically to S-methadone and the first PBP domain comprises an amino acid sequence of positions 1-75 of SEQ ID NO: 4 or SEQ ID NO: 25 and the second PBP domain comprises an amino acid sequence of positions 325-521 of SEQ ID NO: 4 or SEQ ID NO: 25. In some embodiments, the at least one amino acid substitution mutation comprises a substitution mutation of N11E or a substitution mutation homologous to N11E. In some embodiments, the polypeptide or the opioid biosensor comprises substitution mutations of N11E and F436A and the opioid biosensor binds specifically to Tapentadol. In some embodiments, the polypeptide or the opioid biosensor comprises at least one additional substitution mutation selected from: K10I, Q15G, T43E, T68H, T325S, K330G, D341R, Y357G, A360T, E391F, R395G, V405L, H455A, D490L, and a combination thereof.

In some embodiments, the polypeptide or the opioid biosensor binds specifically to Tapentadol and the first PBP domain comprises an amino acid sequence of positions 1-75 of SEQ ID NO: 3 or SEQ ID NO: 24 and the second PBP domain comprises an amino acid sequence of positions 325-521 of SEQ ID NO: 3 or SEQ ID NO: 24. In some embodiments, the polypeptide or the opioid biosensor comprises a Gly at position 357. In some embodiments, the polypeptide or the opioid biosensor further comprising a substitution mutation of F436W. In some embodiments, the polypeptide or the opioid biosensor comprises a substitution mutation of F436W, a Asn at position 11 and a Gly at position 357 and the opioid biosensor binds specifically to fentanyl. In some embodiments, the polypeptide or the opioid biosensor comprises at least one additional substitution mutation selected from: K10A, Q15G, T43G, T68H, T325S, K330G, D341R, Y357G, A360T, E391F, R395A, V405L, H455A, D490G, D490L, and a combination thereof. In some embodiments, the polypeptide or the opioid biosensor binds specifically to Fentanyl and the first PBP domain comprises an amino acid sequence at least 90% identical to position 1-75 of SEQ ID NO: 1, SEQ ID NO: 2 or SEQ ID NO: 23 and the second PBP domain comprises an amino acid sequence at least 90% identical to positions 325-521 of SEQ ID NO: 1, SEQ ID NO: 2 or SEQ ID NO: 23. In some embodiments, the polypeptide or the opioid biosensor binds specifically to fentanyl and the first PBP domain comprises an amino acid sequence of position 1-75 of SEQ ID NO: 1, SEQ ID NO: 2 or SEQ ID NO: 23 and the second PBP domain comprises an amino acid sequence of positions 325-521 of SEQ ID NO: 1, SEQ ID NO: 2 or SEQ ID NO: 23.

The polypeptide or the opioid biosensor can comprise a reporter connected to the first PBP domain and the second PBP domain on either end. In some embodiments, the reporter is connected to the first PBP domain, the second PBP domain, or both via one or more linkers. In some embodiments, the reporter is a fluorescent protein, optionally a superfolder GFP. In some embodiments, the reporter comprises a TYG (Thr-Tyr-Gly) chromophore. In some embodiments, the reporter has an amino acid sequence of positions 79-320 of a sequence selected from SEQ ID NOs: 1-28 or an amino acid sequence having at least 90% sequence identity to position 75-320 of a sequence selected from SEQ ID NOs: 1-28. In some embodiments, the one or more linkers are about 4 amino acid in length, and optionally wherein the one or more linkers have a sequence selected from: FPEP, PPPS, PPPI, PPPA, PPPM, PPPV, APPP, and EPPS. In some embodiments, the biosensor comprises a first linker connects the first PBP domain with the reporter and a second linker connects the reporter with the second PBP domain; and optionally wherein the first linker has the amino acid sequence of FPEP and the second linker has an amino acid sequence selected from: PPPS, PPPI, PPPA, PPPM, PPPV, APPP, and EPPS.

Disclosed herein include nucleic acids. In some embodiments, a nucleic acid encoding the polypeptide or the opioid biosensor disclosed herein. In some embodiments, the vector is a viral vector or a plasmid. In some embodiments, the vector is an AAV vector.

Disclosed herein include a cell, tissue, tissue, organ, or host, comprising the polypeptide or the opioid biosensor the nucleic acid disclosed herein, the vector of disclosed herein, or a combination thereof. In some embodiments, wherein (1) the cell, tissue or organ is from or derived from an animal or (2) the host is an animal, optionally the animal is a mammal, and further optionally the mammal is a human, mice or rat.

Disclosed herein include a kit, comprising (1) one or more of the polypeptide or the opioid biosensor the vector disclosed herein; (3) the cell, tissue, organ or host disclosed herein; or a combination thereof.

Disclosed herein include a device comprising one or more of the polypeptide or the opioid biosensor the device disclosed herein, wherein the polypeptide or the opioid biosensor is attached to one or more test strips. In some embodiments, the device is a wearable device.

Disclosed herein include a method of detecting an opioid in a sample, the method comprising: providing a polypeptide or a opioid biosensor or the device disclosed herein; contacting the polypeptide, opioid biosensor or the device with a sample suspected of containing an opioid; and detecting the conformational change that can be trigged by the binding of the opioid, thereby determining the presence or absence of the opioid in the sample. In some embodiments, detecting the conformational change of the opioid biosensor comprises detecting a signal emitted by the opioid biosensor, and optionally the signal is emitted by the reporter of the opioid biosensor. In some embodiments, the signal comprises a level or an intensity of fluorescence emitted by the reporter of the opioid biosensor. In some embodiments, the signal comprises a signal decrease or increase in a signal emitted by the reporter upon the opioid biosensor binding to an opioid. In some embodiments, the method comprises correlating the level of fluorescence with a concentration of opioid.

In some embodiments, detecting the signal emitted by the reporter of the opioid biosensor comprises obtaining a S-slope of an opioid biosensor and an opioid. In some embodiments, the method comprises contacting the opioid biosensor with a target environment prior to the detecting the signal emitted by the reporter of the opioid biosensor. The target environment can comprise a cell sample, a cell line, a tissue sample, an organ, a body fluid, an environment sample, or a combination thereof. In some embodiments, the target environment comprises a biological fluid, for example the biological fluid comprises a bodily fluid.

In some embodiments, the biological fluid comprises sweat, serum, urine, blood, saliva, or a combination thereof. In some embodiments, the opioid is an opioid specific to a kappa opioid receptor, delta opioid receptor, or mu opioid receptor. In some embodiments, the opioid is an opioid selected from codeine, morphine, morphainan, fentanyl, phenyl piperazine, hydrocodone, oxycodone, oxymorphone, heroin, and benzenoid monoamine. In some embodiments, the opioid is present in the sample at a concentration no more than 20 μM. In some embodiments, the detecting comprises continuous monitoring the opioid in the sample.

### BRIEF DESCRIPTION OF THE DRAWINGS

FIG. 1A depicts an exemplary scheme for biosensor mechanism: in the unbound state, reporter moiety (e.g., GFP's chromophore) has a poor chemical environment for fluorescence. OpuBC binds S-methadone with a “Venus flytrap” conformational change that increases the brightness of the GFP chromophore. FIG. 1B depicts a non-limiting embodiment of a crystal structure of iNicSnFR3a (PDB: 7S7T) virtually mutated to iS-methadoneSnFR (mutations shown in dark gray spheres). All but one putative cation-π residue in iNicSnFR3a were maintained in iS-methadoneSnFR's binding pocket (including F12, Y65, and Y357, shown in spheres). The one aromatic mutation was a conservative W436F.

FIG. 2 shows a non-limiting heat map visualization of the “opioid panel x biosensor library” screen. ΔF/F₀ values 0-10 are coded on a color scale ranging from white to dark gray. Rows are organized by opioid class; p opioids are further classified by their structure subclass. Columns are organized by the biosensor evolution campaigns toward detecting other ligand classes.

FIG. 3 depicts an exemplary evolution history of fentanyl biosensors starting with the hit “AK1” and ending with the full characterization of “iFentanylSnFR2.0” in ˜10 months.

FIG. 4A shows an exemplary plot of purified iFentanylSnFR2.0 response vs. [fentanyl] (i.e. fentanyl concentration). The exemplary fentanyl biosensor has SEQ ID NO: 2. FIG. 4B shows an exemplary plot of purified iS-methadoneSnFR1.0 response vs. [S-methadone] (i.e. S-methadone concentration). The exemplary S-methadone biosensor has SEQ ID NO: 4. FIG. 4C shows an exemplary plot of purified iTapentadolSnFR1.0 response vs. [tapentadol] (i.e. tapentadol concentration). The exemplary tapentadol biosensor has SEQ ID NO: 3. FIG. 4D shows an exemplary plot of purified iLevorphanolSnFR1.0 response vs. [levorphanol] (i.e. levorphanol concentration). The exemplary levorphanol biosensor has SEQ ID NO: 5.

FIG. 5A-FIG. 5C show exemplary isothermal titration calorimetry data plot for (FIG. 5A) fentanyl vs iFentanylSnFR2.0; (FIG. 5B) S-methadone vs iS-methadoneSnFR1.0; and (FIG. 5C) tapentadol vs iTapentadolSnFR1.0.

FIG. 6A-FIG. 6D show non-limiting ΔF/F₀ plots of exemplary biosensor iFentanylSnFR2.0 (FIG. 6A), iS-methadoneSnFR1.0 (FIG. 6B), iTapentadolSnFR1.0 (FIG. 6C), and iLevorphanolSnFR1.0 (FIG. 6D) against an endogenous peptide met-enkephalin.

FIG. 7A-FIG. 7D show non-limiting ΔF/F₀ plots of exemplary biosensor iFentanylSnFR2.0 (FIG. 7A), iS-methadoneSnFR1.0 (FIG. 7B), iTapentadolSnFR1.0 (FIG. 7C), and iLevorphanolSnFR1.0 (FIG. 7D) against various neurotransmitters.

FIG. 8A-FIG. 8E show non-limiting ΔF/F₀ plots of exemplary biosensor iFentanylSnFR2.0 (FIG. 8A-FIG. 8B), iS-methadoneSnFR1.0 (FIG. 8C), iTapentadolSnFR1.0 (FIG. 8D), and iLevorphanolSnFR1.0 (FIG. 8E) against other opioids.

FIG. 9A-FIG. 9B show non-limiting plots of biosensor iFentanylSnFR2.0 dose response against fentanyl in mouse serum (FIG. 9A) and human sweat (FIG. 9B). FIG. 9C shows non-limiting plots of biosensor iS-methadoneSnFR1.0 dose response against S-methadone in human sweat, human saliva and mouse serum.

FIG. 10 shows an exemplary dose-response comparison between iFentanylSnFR1.0 490G solution stored at −80° C. and lyophilized powder stored at room temperature for 3 weeks in the dark and then dissolved in solution.

FIG. 11A-FIG. 11C shows exemplary dose responses of iFentanylSnFR1.0 localized to the PM or the ER of live HeLa cells during bath perfusion of fentanyl. FIG. 11D shows a highly sensitive dose response that uses iFentanylSnFR2.0-PM in HeLa.

FIG. 12A-FIG. 12D shows exemplary time-resolved signal and dose response of iTapentadolSnFR1.0 at the PM (FIG. 12A-FIG. 12B) and in the ER (FIG. 12C-FIG. 12D) of HeLa cells.

FIG. 13A-FIG. 13H show stopped-flow kinetics measured for four exemplary biosensors: iFentanylSnFR2.0 (FIG. 13A-FIG. 13B), iS-methadoneSnFR1.0 (FIG. 13C-FIG. 13D), iTapentadolSnFR1.0 (FIG. 13E-FIG. 13F), and iLevorphanolSnFR1.0 (FIG. 13G-FIG. 13H).

FIG. 14 shows the GFP fluorescence imaging of brain tissue injected with iFentanylSnFR2.0 under drug perfusion. The about 1 mm×1 mm injection location is outlined in gray square.

FIG. 15 shows the GFP fluorescence imaging of individual neuron cytoplasm responding to bath perfused fentanyl.

FIG. 16A-FIG. 16K show dose responses of exemplary biosensor leads tested against various opioids and analogs.

FIG. 17A illustrates an exemplary directed evolution strategy. FIG. 17B shows fluorescence responses of exemplary S-methadone biosensors to S-methadone. FIG. 17C shows the S-slope of iNicSnFR3a and iS-methadoneSnFR. All dose responses conducted in 3×PBS pH 7.0, n=3, SEM as error bars.

FIG. 18 shows the chiral resolution of racemic methadone (R-methadone and S-methadone).

FIG. 19 shows the fluorescence responses of OpuBC biosensors against methadone enantiomer.

FIG. 20 shows an exemplary evolution tree from iNicSnFR3a to iS-methadoneSnFR.

FIG. 21 illustrates S-methadone docked into iNicSnFR3a (PDB: 7S7T) (panel A) and the fluorescence dose response of various S-methadone biosensor variants (panels B-C).

FIG. 22 shows the fluorescence dose response of iS-methadoneSnFR vs other clinically used opioids

FIG. 23 shows plots of stopped flow kinetics for racemic methadone (left panel: raw data; right panel: 1 sec. steady-state fit).

FIG. 24 shows exemplary R-methadone titration plots in S-methadone response.

FIG. 25 shows pH-dependent iS-methadoneSnFR dose response against S-methadone.

FIG. 26A shows a non-limiting spinning disc confocal imaging of HeLa cells transfected with iS-methadoneSnFR_PM, _ER, and _Golgi (470 nm excitation, 535 nm emission, 100×1.4 NA objective). FIG. 26B shows exemplary time-resolved waveforms during “stuttered step” perfusion (1 min drug on, 1 min wash and each dose applied twice) during fluorescence imaging (40×, 1.0 NA 470 nm excitation). PM n=11; ER n=10; Golgi n=11.4 Hz framerate was smoothed by 4-frame adjacent averaging. SEM given as light-colored bounds. FIG. 26C shows exemplary S-slope plotted by organelle for response across 0-250 nM [S-methadone]. Response at each dose was averaged across the cells and corrected for the HBSS artifact (constant subtraction across all responses). SEM given as error bars.

FIG. 27A shows a non-limiting spinning disc confocal imaging of mouse hippocampal neuronal culture transduced with PHP.eb-hSyn-iS-methadoneSnFR-PM-WPRE. FIG. 27B shows exemplary time-resolved waveforms during bath perfusion of S-methadone (n=12, SEM as gray bounds). HBSS-corrected dose response Hill parameters were given.

FIG. 28A-FIG. 28B show dose response with mTurquoise variants of iFentanylSnFR2.0 and iTapentadolSnFR1.0 respectively.

FIG. 29 show sequences of exemplary opioid biosensors described herein.

FIG. 30 depicts X-ray crystallographic structure of an exemplary opioid biosensor iFentanylSnFR2.0 Panel (A): full length biosensor with a N-terminal His6-tag, 90% of residues identified in molecular replacement. Panel (B): residues at the opioid binding pocket.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Opioid use poses significant risks to human health. Opioid use disorder is the chronic use of opioids that causes clinically significant distress or impairment. Opioid use disorder affects over 16 million people worldwide, over 2.1 million in the United States, and there are over 120,000 deaths worldwide annually attributed to opioids. The rise of opioid use disorder and death by overdose necessitates new tools for both neuropharmacology and clinical diagnostic methods. Managing opioid use disorder is however complicated by incomplete knowledge of cellular tolerance mechanisms and variability in individual responses to opioids due to genetic variations, health condition, and environment.

The pharmacokinetics of opioids can be defined at the subcellular level which addresses cellular tolerance and at the whole-body level which provides the means to personalize opioid regimens. Currently, pharmacokinetic measurements of opioid in humans are rare and laborious. Several minimally invasive technologies exist for continuous analyte monitoring for endogenous analytes, but no suitable opioid-binding moieties exist to provide continuous opioid monitoring.

Disclosed herein include engineered biosensors, compositions, vectors, cells, systems, and methods that provide opioid biosensors with sensitivity and selectivity suitable for continuous opioid monitoring as well as the use of the opioid biosensors for detecting one or more specific opioids. In particular, the biosensors herein described are engineered to have improved sensitivity for an opioid ligand in its pharmacologically relevant concentration range, sensitivity against endogenous molecules, selectivity against exogenous drugs, including those of the same drug class, photostability for the duration of measurements, as well as physical stability outside cells and aqueous solution. The methods described herein also establish a general method of directed evolution toward sensitive and selective opioid biosensors.

Disclosed herein includes an opioid biosensor (e.g., nucleic acids, proteins, vectors, cells and kits). The opioid biosensor can comprise a first mutated periplasmic binding protein (PBP) domain and a second PBP domain connected to the first PBP domain. The opioid biosensor is capable of undergoing a detectable conformational change upon binding to an opioid. At least one of the first and second PBP domain comprises at least one amino acid substitution mutation selected from the positions functionally equivalent to K10, N11, Q15, T43, T68, T325, K330, D341, Y357, A360, E391, R395, V405, F436, H455, or D490 of an amino acid sequence of SEQ ID NO: 29. The opioid biosensor can further comprise a reporter connected to the first PBP domain and the second PBP domain on either end.

Disclosed herein also includes a method of detecting an opioid. The method can comprise detecting a signal emitted by the reporter of the opioid biosensor. The signal can comprise a signal decrease or increase in a signal emitted by the reporter upon the opioid biosensor binding to an opioid. The method can be used to detect an opioid in a target environment comprising a cell sample, a cell line, a tissue sample, an organ, a body fluid (e.g., sweat, serum, urine, blood, saliva), an environment sample, or a combination thereof.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The terms “nucleic acid” and “polynucleotide” also specifically include nucleic acids composed of bases other than the five biologically occurring bases (adenine, guanine, thymine, cytosine and uracil).

Unless specified otherwise, the left-hand end of any single-stranded polynucleotide sequence discussed herein is the 5′ end; the left-hand direction of double-stranded polynucleotide sequences is referred to as the 5′ direction.

The term “naturally occurring” as used herein refers to materials which are found in nature or a form of the materials that is found in nature.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990, the content of each of which is incorporated herein in its entirety.

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Arginine and lysine are considered as functionally equivalent residues to histidine.

The phrase “substantially identical,” in the context of two nucleic acids or polypeptides (e.g., nucleic acids encoding a biosensor or a portion thereof, or the amino acid sequence of a biosensor or a portion thereof) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, or over the full length of the two sequences to be compared.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

As used herein, the term “vector” can refer to a vehicle for carrying or transferring a nucleic acid. Non-limiting examples of vectors include viral vectors (for example, adenovirus vectors, adeno-associated virus (AAV) vectors, retrovirus vectors, lentiviral vectors, herpes virus vectors, phages, and poxvirus vectors); non-viral vectors such as liposomes, naked DNA, plasmids, cosmids; and the like.

As used herein, the term “construct” refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

As used herein, the term “element” refers to a separate or distinct part of something, for example, a nucleic acid sequence with a separate function within a longer nucleic acid sequence. The terms “transcription regulatory element” and “expression control element” are used to refer to nucleic acid molecules that can influence the expression (including at the transcription and/or translation level) of an operably linked coding sequence in a specific host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell. The promoter can be a specific promoter, e.g., cell type-specific and/or tissue-specific. The promoter can be constituent or inducible (e.g., by chemical agent, biological agent, temperature, and/or pH).

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

### Opioid Biosensor

Provided herein include genetically engineered opioid biosensors, i.e. nucleic acids encoding peptides and/or the encoded peptides for use as opioid biosensors. An opioid biosensor herein described can be employed in biological systems in vivo, in vitro, or ex vivo to detect and/or monitor opioids with improved selectivity and sensitivity due to its capability to undergo a detectable conformational change upon binding to its respective opioid ligand(s).

The term “opioid” includes naturally occurring compounds derived from opium as well as semisynthetic and/or synthetic compounds capable of modulating opioid receptors including, for example, mammalian μ, δ, and κ-opioid receptors. Mammalian μ, δ, and κ-opioid receptors transduce opioid ligand binding in defined circuits to elicit analgesia and reward (Darcq and Kieffer '18). Therefore, opioid can be categorized according to the type of opioid receptor it binds to. Exemplary opioids that can be detected by the biosensors herein described include, but are not limited to, κ opioids including U-69,593, salvinorin B, salvinorin A, nor-Binaltorphimine, ML138, GR-89696, BRL-52537, amentoflavone, δ opioids including ARM390, SNC80, naltrindole, naltriben, desmethylclozapine, 6′ guanidinonaltrindole (6′-GNTI), tianeptine, p opioids including codeine, oxymorphone, nalorphine, hydromorphone, morphine-6-glucuronide, hydrocodone, naltrexone, nalmefene, oxycodone, noroxymorphone, butorphanol, levallorphan, levorphanol, alfentanil, sufentanil, fentanyl, carfentanil, norfentanyl, meperidine, ML-335, loperamide, meptazinol, mitragynine, tapentadol, tramadol, desMe-Tramadol, BMS-986122, methadone (racemic) R-methadone, and S-methadone. Opioids that can be detected by the biosensors herein described can also include others identifiable to a person skilled in the art. In some embodiments, the opioid is an opioid specific to a kappa opioid receptor, delta opioid receptor, or mu opioid receptor. In some embodiments, the opioid is codeine, morphine, morphinan, fentanyl, phenyl piperazine, hydrocodone, oxycodone, oxymorphone, heroin, or benzenoid monoamine.

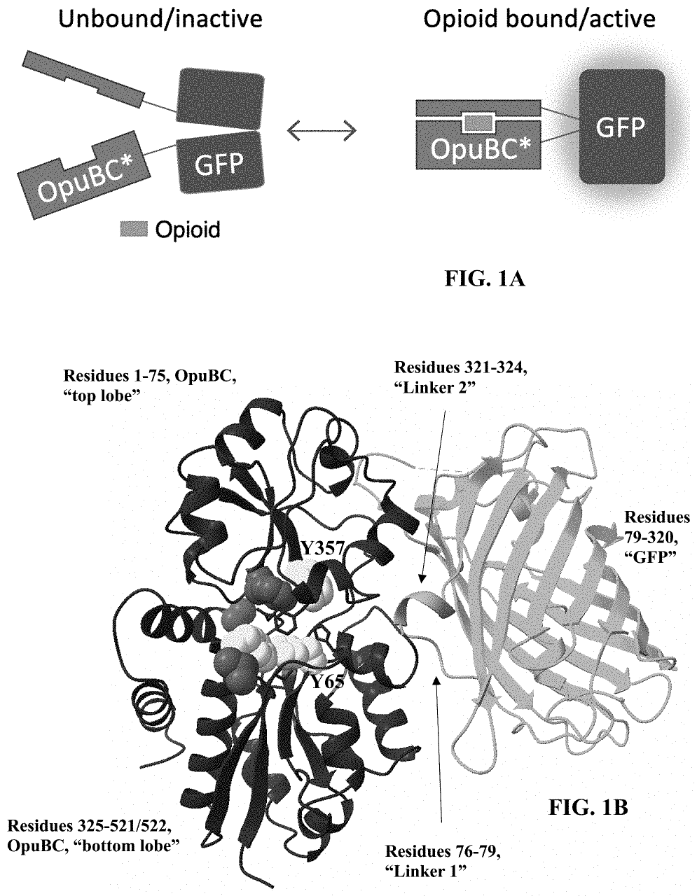

In some embodiments, the opioid biosensor herein described can comprise an opioid binding domain that can undergo a conformational change upon binding to an opioid (see, for example, FIG. 1A). When the opioid binding domain is attached to a reporter (e.g., a fluorescence protein), the conformational change of the opioid binding domain can allosterically modulate or regulate signaling by the reporter. For example, as shown in FIG. 1A, a mutated OpuBC clamps S-methadone in a “Venus flytrap” conformational change, thereby enforcing a highly fluorescent GFP chromophore to emit a signal.

In some embodiments, the opioid biosensor (e.g., opioid binding domain) comprises a modified periplasmic binding protein (PBP) or a portion or a variant thereof. PBPs are nonenzymatic receptors that bacteria use to sense small molecules and transport them into the cytoplasm. Most PBPs participate in the transport of solute molecules into the cytoplasm via ABC (ATP-binding cassette) transporters. These proteins can mediate a wide variety of fundamental processes including transport, chemotaxis, quorum sensing and other signaling pathways. The structure of PBPs is composed of two subdomains (or lobes), each consisting of a central beta-sheet and surrounding alpha-helices, linked by a rigid alpha-helix. The substrate binding site is located in a cleft between the two alpha/beta subdomains. Accordingly, the PBP domain of the biosensors herein described can comprise two subdomains: a first PBP domain and a second PBP domain. In some embodiments, the PBPs are amine-binding PBPs. In some embodiments, the PBPs can bind primary, secondary, or tertiary amine with molecular weight in a range from 100 to 600 Da or greater.

An opioid biosensor herein disclosed can comprise a first PBP domain and a second PBP domain connected to the first mutated PBP domain wherein at least one of the first and second PBP domain is a mutated PBP domain and wherein the opioid biosensor is capable of undergoing a detectable conformational change upon binding to an opioid. The connection between the first PBP domain and the second PBP domain can be direct (e.g., via a peptide bond between the N-terminal of one PBP domain and the C-terminal of the other PBP domain) or indirect via one or more linkers and other components or moiety (e.g., a reporter).

The PBP domains of an opioid biosensor herein described can be homologous to an OpuBC or a variant thereof (e.g., OpuBC from Thermoanaerobacter sp. X513 or its variants). The OpuBC or variants thereof can be a choline-binding protein or an OpuBC modified to detect ligands other than choline.

In some embodiments, the PBP domain(s) of the biosensors herein described is homologous to a OpuBC from Thermoanaerobacter sp. X513 having a SEQ ID NO: 28. The OpuBC from Thermoanaerobacter sp. X513 is a hyperthermophilic homologue of Bacillus subtilis OpuBC. B. subtilis can synthesize the compatible solute glycine betaine as an osmoprotectant from an exogenous supply of the precursor choline. Import of choline is mediated by two osmotically inducible ABC transport systems: OpuB and OpuC. OpuC catalyzes the import of various osmoprotectants, whereas OpuB is highly specific for choline. OpuBC is the substrate-binding protein of the OpuB transporter. In some embodiments, the PBP domains of an opioid biosensor herein described comprise sequences homologous to the PBP domains of OpuBC having SEQ ID NO: 28. Residues 1-75 of SEQ ID NO: 28 corresponds to a first PBP domain and residues 76-272 of SEQ ID NO: 28 corresponds to a second PBP. Therefore, in some embodiments, the first PBP domain of an opioid biosensor herein described can be homologous to residues 1-75 (the first PBP domain) of SEQ ID NO: 28 and the second PBP domain of an opioid biosensor can be homologous to residues 76-272 (the second PBP domain) of SEQ ID NO: 28. Table 1 provides the sequences of the first PBP domain and the second PBP domain of SEQ ID NO: 28.

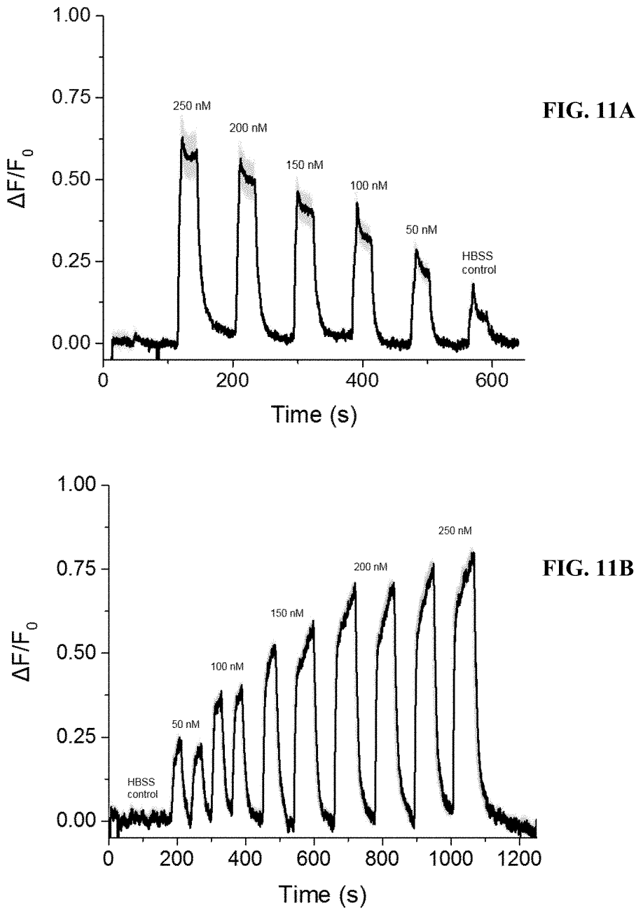

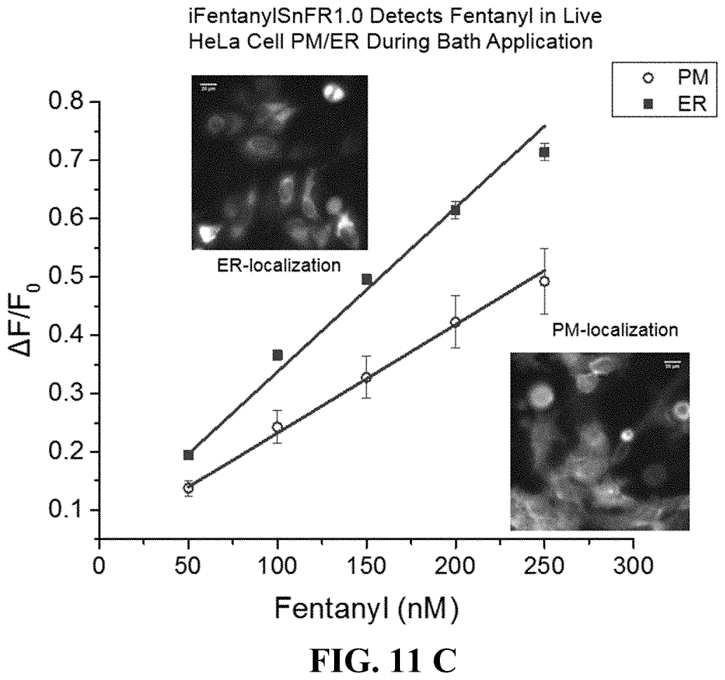

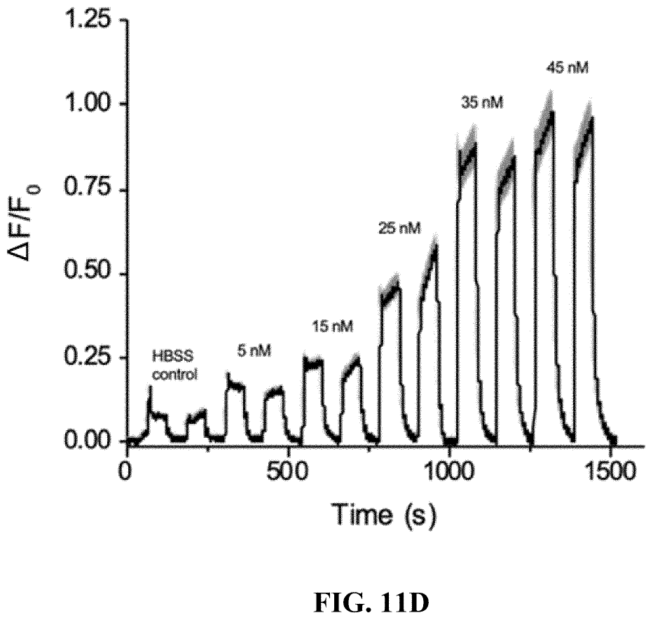

In some embodiments, an OpuBC or a variant thereof can comprise the PBP domain of iNicSnFR1 (SEQ ID NO: 26) which has been engineered to selectively bind nicotine. iNicSnFR1 comprises a PBP including a first PBP domain (positions 1-75 of SEQ ID NO: 26) and a second PBP domain (positions 325-522 of SEQ ID NO: 26) attached to a reporter via two linkers. In other embodiments, an OpuBC or a variant thereof can comprise the PBP domain of iNicSnFR3a (SEQ ID NO: 28) which has been engineered to selectively bind nicotine and acetylcholine. iNicSnFR3a comprises a PBP domain including a first PBP domain (positions 1-75 of SEQ TD NO: 27) (see, for example, top lobe in FIG. 1B) and a second PBP domain (positions 325-521 of SEQ ID NO: 27) (see, for example, bottom lobe in FIG. 1B) attached to a reporter (see, for example, GFP in FIG. 1B) via two linkers (see, for example, linker 1 and linker 2 in FIG. 11B). Sequences of exemplary PBP domains are provided in Table 1.

TABLE 1
Sequences of exemplary first PBP
and second PBP domains.

| | | SEQ |
|---|---|---|
| | | ID |
| Biosensor | Sequence | NO |
| First PBP of OpuBC | ANDTVVVGSKNFTEQIIVA | 30 |
| from Thermoanaerobacter | NMVAEMIEAHTDLKVVRKL | |
| sp. X513 (residues 1-75 | NLGGTNVNFEAIKRGGANN | |
| of SEQ ID NO: 28) | GIDIYVEYTGTGLVDILG | |
| Second PBP of OpuBC | TTDPEKAYETVKKEYKDKW | 31 |
| from Thermoanaerobacter | NIVWLKPLGFNNTYTLAVK | |
| sp. X513 (residues 76- | DELAKQYNLKTFSDLAKIS | |
| 272 of SEQ ID NO: 28) | DKLILGATMEFLERPDGYP | |
| | GLQKVYNFKFKHTKSMDMG | |
| | IRYTAIDNNEVQVIDAFAT | |
| | DGLLVSHKLKILEDDKHFF | |
| | PPYYAAPIIRQDVLDKHPE | |
| | LKDVLNKLANQISDEEMQK | |
| | LNYKVDGEGQDPAKVAKEF | |
| | LKEKGLI | |
| First PBP of iNicSnFR1 | ANDTVVVGSINFTEQIIVA | 32 |
| (residues 1-75 of | NMVAEMIEAHTDLKVVRKL | |
| SEQ ID NO: 26) | NLGGTNVNFEAIKRGGANN | |
| | GIDIYVEYTGTGLVDILG | |
| Second PBP of iNicSnFR1 | STDPEGAYETVKKEYKRKW | 33 |
| (residues 325-521 of | NIVWLKPLGFNNTYTLAVK | |
| SEQ ID NO: 26) | DELAKQYNLKTFSDLAKIS | |
| | DKLILGATMFFLEKPDGYP | |
| | GLQKLYNFKFKHTKSMDMG | |
| | IRYTAIDNNEVQVIDAFAT | |
| | DGLLVSHKLKILEDDKHFF | |
| | PPYYAAPIIRQDVLDKHPE | |
| | LKDVLNKLANQISDEEMQK | |
| | LNYKVDGEGQDPAKVAKEF | |
| | LKEKGLIL | |
| First PBP of iNicSnFR3a | ANDTVVVGSINFTEGIIVA | 34 |
| (residues 1-75 of | NMVAEMIEAHTDLKVVRKL | |
| SEQ ID NO: 27) | NLGGENVNFEAIKRGGANN | |
| | GIDIYVEYTGHGLVDILG | |
| Second PBP of iNicSnFR3a | STDPEGAYETVKKEYKRKW | 35 |
| (residues 325-521 of | NIVWLKPLGFNNTYTLTVK | |
| SEQ ID NO: 27) | DELAKQYNLKTFSDLAKIS | |
| | DKLILGATMFFLEGPDGYP | |
| | GLQKLYNFKFKHTKSMDMG | |
| | IRYTAIDNNEVQVIDAWAT | |
| | DGLLVSHKLKILEDDKAFF | |
| | PPYYAAPIIRQDVLDKHPE | |
| | LKDVLNKLANQISLEEMQK | |
| | LNYKVDGEGQDPAKVAKEF | |
| | LKEKGLI | |

As used herein, proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 2500 sequence similarity over 50, 100, 150 or more residues is routinely used to establish homology. Higher levels of sequence similarity, e.g., 6000, 7000, 8000, 9000, 9500, or 990% or more, can also be used to establish homology. Methods for determining sequence similarity percentages (e.g., BLASTP and BLASTN using default parameters) are described herein and are generally available. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85.2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

For sequence comparison and homology determination, typically one sequence acts as a reference sequence to which test sequences are compared. When using a sequence comparison algorithm, query and reference sequences are input into a computer, subsequence coordinates are designated, if necessary, and sequence algorithm program parameters are designated. The sequence comparison algorithm then calculates the percent sequence identity for the query sequence(s) relative to the reference sequence, based on the designated program parameters as will be understood by a person skilled in the art.

In some embodiments, an opioid biosensor can, for example, comprise, or consist of, an amino acid sequence having at least 80% sequence identity to an amino acid sequence of any of the PBP domains disclosed herein (e.g., PBP domains from a biosensor having an amino acid sequence of any one of SEQ ID NOs: 1-29). For example, the opioid biosensor can comprise or consist of an amino acid sequence having at least 80% (about, at least, at least about, at most, or at most about 80%, 81%, 82%, 83%, 84%, 85%, 86%, 87%, 88%, 89%, 90%, 91%, 92%, 93%, 94%, 95%, or a number between any two of the values) sequence identity to an amino acid sequence of the PBP domains of SEQ ID NO: 26, SEQ ID NO: 27, or SEQ ID NO: 29. The first PBP domain of the opioid biosensor can comprise or consist of an amino acid sequence having at least 80% sequence identity to positions 1-75 of SEQ ID NO: 26, SEQ ID NO: 27, or SEQ ID NO: 29. The second PBP domain of the opioid biosensor can comprise or consist of an amino acid sequence having at least 80% sequence identity to positions 325-521 of SEQ ID NO: 26, SEQ ID NO: 27 or SEQ ID NO: 29. In some embodiments, the first PBP domain of the opioid biosensor can comprise or consist of an amino acid sequence having at least 80% sequence identity to positions 1-75 of SEQ ID NO: 29 and the second PBP domain of the opioid biosensor can comprise or consist of an amino acid sequence having at least 80% sequence identity to positions 325-522 of SEQ ID NO: 29. In some embodiments, the sequence variation are within the binding pocket of the PBP domains. Such variation may affect the selectivity and/or specificity of an opioid biosensor. In some embodiments, the sequence variation are not within the binding pocket of the PBP domains (e.g., in the solvent exposed surface areas). Such sequence variations may not affect the selectivity and/or specificity of an opioid biosensor.

In some embodiments, an opioid biosensor comprises or consists of an amino acid sequence having, or having about, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to an amino acid sequence of any of the PBP domains of an opioid biosensor having a sequence selected from the group consisting of SEQ ID NOs: 1-25.

In some embodiments, an opioid biosensor comprises, or consists of, an amino acid sequence having at least, or at least about, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, sequence identity to an amino acid sequence of any of the PBP domains of an opioid biosensor having a sequence selected from the group consisting of SEQ ID NOs: 1-25.

In some embodiments, an opioid biosensor comprises, or consists of, an amino acid sequence having at most, or at most about, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, sequence identity to an amino acid sequence of any of the PBP domains of an opioid biosensor having a sequence selected from the group consisting of SEQ ID NOs: 1-25.

In some embodiments, the first PBP of an opioid biosensor comprises, or consists of, an amino acid sequence having, having about, having at least, having at least about, having at most, or having at most about 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to the amino acid sequence of positions 1-75 of any of SEQ ID NOs:1-25. In some embodiments, the second PBP of the biosensor comprises, or consists of, an amino acid sequence having, having about, having at least, having at least about, having at most, or having at most about 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to the amino acid sequence of positions 325-521 of any of SEQ ID NOs: 1-25. In some embodiments, an opioid biosensor comprises or consists of an amino acid sequence selected from the group consisting of SEQ ID NOs:1-25.

The opioid biosensor can, for example, comprise, or consists of, an amino acid sequence having, having about, having at most or having at most about one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, mismatch compared to an amino acid sequence of any of the PBP domains of any of the biosensor disclosed herein (e.g., any of the PBP domains of the biosensors having an amino acid sequence of any one of SEQ ID NOs: 1-27 or SEQ ID NO: 29).

The opioid biosensor can, for example, comprise, or consists of, a first PBP domain having an amino acid sequence having, having about, having at most or having at most about one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, mismatch compared to an amino acid sequence of the first PBP domain (e.g., positions 1-75) of any of the biosensors or variants disclosed herein (e.g., SEQ ID NOs: 1-27 or SEQ ID NO: 29).

The opioid biosensor can, for example, comprise, or consists of, a second PBP domain having an amino acid sequence having, having about, having at most or having at most about one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, mismatch compared to an amino acid sequence of the second PBP domain (e.g., positions 325-521) of any of the biosensors or variants disclosed herein (e.g., SEQ ID NOs: 1-27 or SEQ ID NO: 29).

In some embodiments, the first PBP domain of an opioid biosensor can comprise or consist of an amino acid sequence having, having about, having at most, or having at most about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty mismatches compared to positions 1-75 of SEQ ID NO: 28. In some embodiments, the second PBP domain of the biosensor can comprise or consist of an amino acid sequence having, having about, having at most, or having at most about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty mismatches compared to positions 76-272 of SEQ ID NO: 28.

The biosensors herein described can comprise one or more additional substitution mutations with reference to a wild type OpuBC protein, which can enhance the selectivity and specificity of the biosensors for one or more specific opioid ligand. In some embodiments, the wild type OpuBC is the OpuBC from Thermoanaerobacter sp. X513 (e.g., SEQ ID NO: 28).

The terms “specificity”, “specific”, “selectivity” or “selective” as used herein with reference the binding between an opioid sensor and an opioid ligand (or a group or category of opioid ligands) refers to the recognition, interaction and formation of a stable complex between the opioid sensor and the opioid ligand (or the group or category of opioid ligands), with substantially less or no recognition, interaction, and formation between the opioid sensor and other opioid or non-opioid molecules that may be present. In some embodiments, an opioid biosensor of the present disclosure specifically binds a target opioid. An opioid biosensor exhibits “specific binding” or “selective binding” if it reacts or associates more frequently, more rapidly, with greater duration and/or with greater affinity with a particular target opioid than it does with alternative targets (e.g., other opioid or non-opioid ligands). For example, an opioid biosensor that specifically (or selectively) binds to S-methadone is an opioid biosensor that binds S-methadone with greater affinity, avidity, more readily, and/or with greater duration than it binds to other opioids or non-opioid targets. In some embodiments, the specific binding is an exclusive binding, that is, an opioid biosensor that specifically binds to a first opioid target exhibit substantially less to no recognition, interaction or formation of a stable complex with a second target. It is also understood by a skilled person upon reading the present disclosure that, for example, an opioid biosensor that specifically binds to a first opioid target may or may not specifically or preferentially bind to a second opioid target. As such, “specific binding” or “selective binding” does not necessarily require (although it can include) exclusive binding. In some embodiments, the sensitivity of a biosensor can be determined by calculating S-slope (ΔF/F₀)/EC₅₀ (e.g., for Hill coefficient ˜1.0) (see, for example, Examples 1, 3, and 5). F₀ provides a baseline fluorescence of the biosensor with no ligand present and ΔF is defined as F_{biosensor+ligand}−F₀, i.e., the difference between the fluorescence of the biosensor bound with a ligand and the fluorescence of the unbound biosensor. EC₅₀ refers to a concentration of an opioid ligand which induces a response halfway between the baseline and maximum after a specified exposure time. In some embodiments, an opioid biosensor herein described shows sensitivity for its opioid ligand when S-slope calculated for the opioid biosensor and the opioid ligand is greater than 0.1, preferably greater than 1.0. In some embodiments, an opioid biosensor herein described is considered as having no sensitivity to a ligand when the S-slope calculated between the opioid biosensor and the ligand is less than 0.1. In some embodiments, the selectivity of an opioid biosensor can be defined in terms of ratios of S-Slopes. A biosensor can be considered selective for a ligand if the S-Slope ratio is about, at least or at least ten times (e.g., 12, 15, 20, 25, 30, 500, 100 or a number between any of the two values) greater for that ligand as compared to any other ligand.

A biosensor's sensitivity to a ligand can be defined in terms of S-slope=Δ(F/F₀)/(Δ[drug]) where [drug]<<the EC₅₀ of that biosensor-ligand pair (Bera '19). S-Slope is given in units of μM⁻¹. This metric is defined as the increase in fluorescence signal from some increase in applied ligand concentration at the beginning of the dose-response relationship. If the Hill coefficient for the entire dose-response relationship is ˜1.0, then S-Slope may be approximated as (ΔFₘₐₓ/F₀)/EC₅₀ where ΔFₘₐₓ/F₀ is the response at saturating [drug] (i.e. dynamic range) and EC₅₀ is the [drug] required to produce a response equal to 12 of the ΔFₘₐₓ/F₀. As used herein, “[drug]” refers to concentration of the drug.

A biosensor's selectivity in binding one ligand compared to another can be stated in terms of S-Slope in response to each ligand. An improvement in sensitivity to a ligand in a mutant biosensor can be stated in terms of increase in S-Slope with respect to that of the parent biosensor.

Opioid biosensors disclosed herein can be used to detect their selective ligand in the pharmacologically relevant range. Therefore, it is advantageous to have a biosensor having a detection limit on the order of magnitude of the lower end of the pharmacologically relevant range. For opioids other than fentanyl, the pharmacologically relevant range can range from about 10 nM to about 1 μM, for example 10 nM, 50 nM, 100 nM, 150 nM, 200 nM, 250 nM, 300 nM, 450 nM, 500 nM, 550 nM, 600 nM, 700 nM, 800 nM, 900 nM, 1000 μM, or a number or a range between any two of these values. For fentanyl, the pharmacologically relevant range ranges from about 1 nM to about 100 nM, for example 1 nM, 5 μM, 10 μM, 20 μM, 30 μM, 40 μM, 50 μM, 60 μM, 70 μM, 80 μM, 90 μM, 100 μM, or a number or a range between any two of these values. The S-Slope determines the lowest detectable [drug] (see Table 4).

TABLE 4
Heuristic Guidelines for cellular experiments
Heuristic Guidelines for cellular experiments

| S-slope | Lowest useful [drug] | |
|---|---|---|
| 0.3 | 0.3 | μM |
| 1 | 0.1 | μM |
| 3 | 10 | nM |
| 10 | 3 | nM |

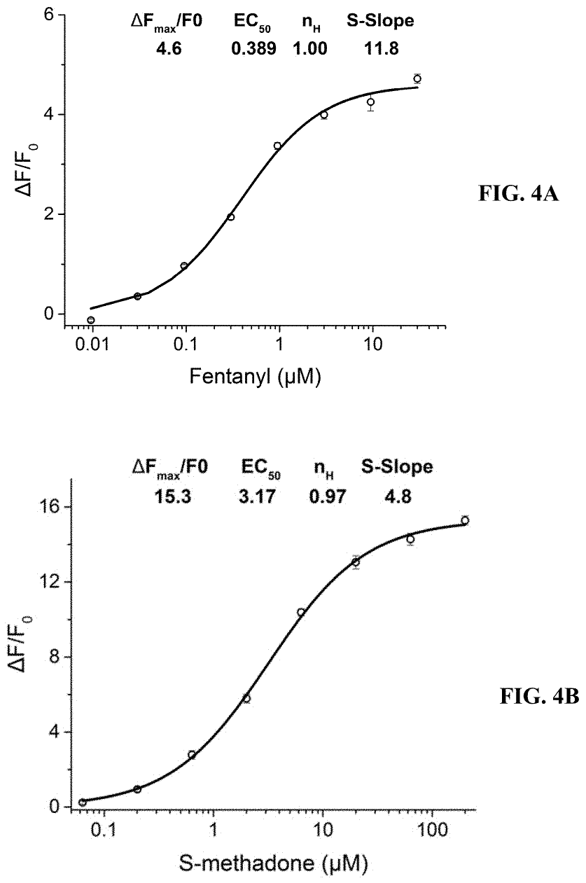

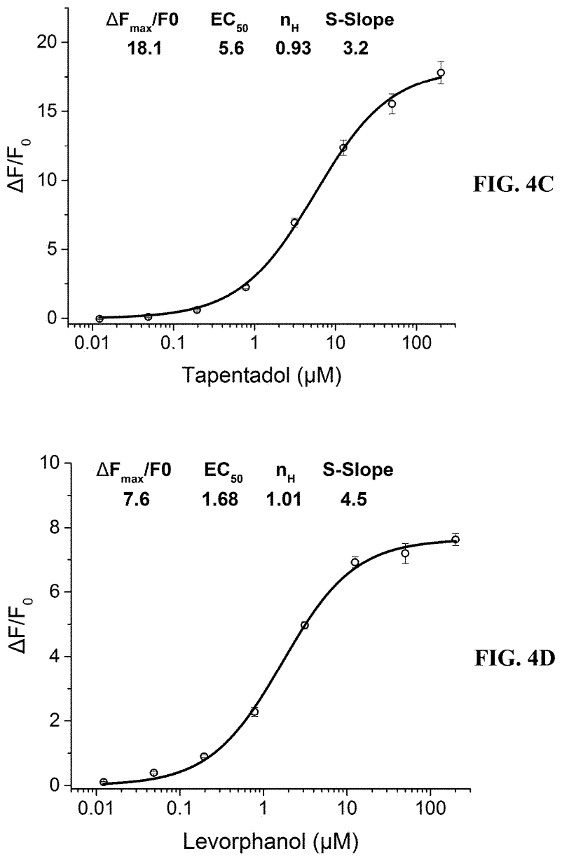

Biosensors disclosed herein allow detection of their selective ligand in the pharmacologically relevant range. For example, as shown herein, iFentanylSnFR2.0, S-Slope=11.8, detection limit on the order of 1 nM (FIG. 4A). iS-methadoneSnFR1.0, S-Slope=4.8, detection limit is 3 to 10 nM (FIG. 4B). iTapentadolSnFR1.0, S-Slope=3.2 (FIG. 4C), detection limit on the order of 10 nM. iLevorphanolSnFR1.0, S-Slope=4.5, detection limit is 3 to 10 nM (FIG. 4D).

In some embodiments, the mutations can be introduced at the binding pocket where the opioid ligand will interact with the opioid binding domain of the biosensor (e.g., PBP domain), such as at the interface between the top lobe and the bottom lobe of the binding moiety (see FIG. 1B, for example). For example, a cation-π residue at the binding pocket can be substituted with a neutral amino acid or an amino acid without an aromatic ring. A cation-π residue typically refers to residues involved in a cation-π interaction, which is a stabilizing electrostatic interaction of a cation (e.g., cationic side chain in amino acids such as Lys or Arg) with the polarizable π electron cloud of an aromatic ring (e.g., in Trp, Tyr or Phe). Therefore, a cation-π interaction can be found when a cationic sidechain or cationic ligand is near an aromatic sidechain. In some embodiments, the substitution mutation herein described can be a cation-π mutation. A cation-π mutation refers to any mutation capable of destabilizing a cation-π interaction, such as substituting a Trp, Tyr or Phe with Ala or Gly or replacing a cationic amino acid with a neutral amino acid or an acidic amino acid. In some embodiments the biosensors herein described can comprise one or more cholinergic-null mutations at cation-π residues (e.g., Y357G mutation in iFentanylSnFR2.0 and WF436A mutation in iTapentadolSnFR1.0). Biosensors with cholinergic-null mutations demonstrate enhanced selectivity against other ligands.

In some embodiments, some of the residues located at the substrate binding pocket are not mutated, such as F12, Y65 of the first PBP domain and/or Y460 of the second PBP domain in SEQ ID NO: 29 and others identifiable by a skilled person upon performing a sequence alignment between the opioid biosensor herein described and the wild type OpuBC.

In some embodiments, a substitution mutation is introduced to a base sequence. A base sequence can be a sequence of a wild type OpuBC (e.g., OpuBC from Thermoanaerobacter sp. X513) or a variant of the wild type OpuBC. In some embodiments, substitution mutations are generated using the wild type OpuBC from Thermoanaerobacter sp. X513 (e.g., SEQ ID NO: 28) as a base sequence. In some embodiments, substitution mutations are generated using a variant of the OpuBC from Thermoanaerobacter sp. X513, such as iNicSnFR1 (SEQ ID NO: 26) and iNicSnFR3a (SEQ ID NO: 27). In some embodiments, an opioid biosensor is generated by introducing one or more substitution mutations into one or both of the PBP domains of Table 1.

In some embodiments, an opioid biosensor can comprise one or more substitution mutations in at least one of the first PBP and second PBP domain at positions functionally equivalent to positions 10, 11, 15, 43, 68, 325, 330, 341, 357, 360, 391, 395, 405, 436, 455 and 490 in SEQ ID No: 29. In some embodiments, the engineered biosensors can comprise one or more substitution mutations in at least one of the first PBP and second PBP domain at positions functionally equivalent to positions 10, 11, 15, 43, 68, 325, 330, 341, 357, 360, 391, 395, 405, 436, 455 and 490 with reference to SEQ ID NOs: 1-27. Any of a variety of substitution mutations at one or more positions functionally equivalent to 10, 15, 43, 68, 325, 330, 341, 357, 360, 391, 395, 405, 436, 455 and 490 which can result in increased selectivity and/or specificity can be made. In some embodiments, the altered opioid biosensor exhibits enhanced specificity and/or selectivity for an opioid ligand (or two or more opioid ligands) as compared to the wild type OpuBC protein from Thermoanaerobacter sp. X513. In some embodiments, an opioid biosensor with one or more substitution mutations herein described exhibit enhanced specificity and/or selectivity as compared to other biosensor lacking the one or more substitution mutations.

The term “functionally equivalent” in the context of substitution mutations refer to mutations that are positionally equivalent or homologous to a given mutation. Generally, functionally equivalent substitution mutations in two or more different biosensors occur at homologous amino acid positions in the amino acid sequences of the biosensors and can have the same functional role in the biosensors. Positionally equivalent or homologous amino acid residues in the amino acid sequences of two or more opioid binding domain (e.g., PBP domains) can be identified using sequence alignment and/or molecular modeling as will be understood by a person skilled in the art.

For example, the substitution mutations can comprise one or more substitution mutations in the first PBP domain at positions 10, 11, 15, 43, and 68 with reference to SEQ ID NOs: 1-27 or SEQ ID No: 29 or at positions functionally equivalent to positions 10, 11, 15, 43, and 68. In some embodiments, position 10 is mutated to Ala, Ile, Val, or Met. In some embodiments, the substitution mutation at position 10 can comprise a mutation of K10A, K10I, K10V, or K10M. In some embodiments, position 11 is substituted to Glu, Val, Pro, Leu, Phe, or Asp. In some embodiments, the substitution mutation at position 11 can comprise a mutation of N11V, N11P, N11E, N11L, N11F, or N11D. In some embodiments, position 15 is substituted to Gly. In some embodiments, the substitution mutation at position 11 can comprise a mutation of Q15G. In some embodiments, position 43 is substituted to Glu, Val, Ileu, or Gly. In some embodiments, the substitution mutation at position 43 can comprise a mutation of T43E, T43V, T43I, T43H, or T43G. In some embodiments, position 68 is substituted to His. In some embodiments, the substitution mutation at position 68 can comprise a mutation of T68H.

For example, the substitution mutations can comprise one or more substitution mutations in the second PBP domain at positions 325, 330, 341, 357, 360, 391, 395, 405, 436, 455 and 490 with reference to SEQ ID NOs: 1-27 or SEQ ID No: 29 or at positions functionally equivalent to positions 325, 330, 341, 357, 360, 391, 395, 405, 436, 455 and 490. In some embodiments, position 325 is mutated to Ser. In some embodiments, the substitution mutation at position 325 can comprise a mutation of T325S. In some embodiments, position 330 is mutated to Gly. In some embodiments, the substitution mutation at position 330 can comprise a mutation of K330G. In some embodiments, position 341 is mutated to Arg. In some embodiments, the substitution mutation at position 341 can comprise a mutation of D341R. In some embodiments, position 357 is mutated to Gly. In some embodiments, position 360 is mutated to Thr. In some embodiments, the substitution mutation at position 360 can comprise a mutation of A360T. In some embodiments, position 391 is mutated to Phe. In some embodiments, the substitution mutation at position 391 can comprise a mutation of E391F. In some embodiments, position 395 is mutated to Ala, Gly, or Met. In some embodiments, the substitution mutation at position 395 can comprise a mutation of R395A, R395G, or R395M. In some embodiments, position 405 is mutated to Leu. In some embodiments, the substitution mutation at position 405 can comprise a mutation of V405L. In some embodiments, position 436 is mutated to Trp, Ala, Thr, or Cys. In some embodiments, the substitution mutation at position 436 can comprise a mutation of F436W, F436A, F436C, or F436T. In some embodiments, position 455 is mutated to Ala, Gly, or Gln. In some embodiments, the substitution mutation at position 455 can comprise a mutation of H455A, H455G, or H455Q. In some embodiments, position 490 is mutated to Leu or Gly. In some embodiments, the substitution mutation at position 490 can comprise a mutation of D490L or D490G. Table 2 provides substitution mutations in exemplary opioid biosensors with respect to SEQ ID NO: 29.

TABLE 2
Substitution mutations in exemplary opioid biosensors.

| | Opioid | |
|---|---|---|
| Biosensor | ligand | Mutations |
| iFentanylSnFR1.0 | Fentanyl | K10A, Q15G, T43E, T68H, T325S, K330G, D341R, Y357G, |
| | | A360T, E391F, R395A, V405L, F436W, H455A, D490L |
| iFentanylSnFR2.0 | Fentanyl | K10A, Q15G, T43G, T68H, T325S, K330G, D341R, Y357G, |
| | | A360T, E391F, R395A, V405L, F436W, H455A, D490G |
| iTapentadolSnFR1.0 | Tapentadol | K10I, N11E, Q15G, T43E, T68H, T325S, K330G, D341R, |
| | | A360T, E391F, R395G, V405L, F436A, H455A, D490L |
| iS-methadoneSnFR1.0 | S-methadone | K10I, N11V, Q15G, T43E, T68H, T325S, K330G, D341R, |
| | | A360T, E391F, R395G, V405L, H455A, D490G |
| iLevorphanolSnFR1.0 | Levorphanol | K10I, N11P, Q15G, T43V, T68H, K330G, D341R, A360T, |
| | | E391F, R395G, V405L, F436W, H455W, D490L |
| iBRL52537SnFR lead 1 | BRL52537 | K10I, N11E, Q15G, T43E, T325S, K330G, D341R, A360T, |
| | | E391F, R395G, V405L, F436W, H455A, D490L |
| iBRL52537SnFR lead 2 | BRL52537 | K10I, N11E, Q15G, T43E, T325S, K330G, D341R, A360T, |
| | | E391F, R395G, V405L, F436W, H455A, D490L |
| iBRL52537SnFR lead 3 | BRL52537 | K10I, N11E, Q15G, T43E, T325S, K330G, D341R, A360T, |
| | | E391F, V405L, F436W, H455A, D490L |
| iTramadolSnFR lead 1 | Tramadol | K10I, N11E, Q15G, T43E, T68H, T325S, K330G, D341R, |
| | (racemic) | A360T, E391F, R395G, V405L, F436T, H455A, D490L |
| iTramadolSnFR lead 2 | Tramadol | K10I, N11E, Q15G, T43E, T68H, T325S, K330G, D341R, |
| | (racemic) | A360T, E391F, R395G, V405L, F436C, H455A, D490L |
| iButorphanolSnFR lead 1 | Butorphanol | K10I, N11E, Q15G, T43E, T325S, K330G, D341R, A360T, |
| | | E391F, R395G, V405L, F436W, H455A, D490L |
| iNorfentanylSnFR lead 1 | Norfentanyl | K10I, N11E, Q15G, T43E, T325S, K330G, D341R, A360T, |
| | | E391F, V405L, F436W, H455A, D490L |
| iSufentanilSnFR lead 1 | Sufentanil | K10I, Q15G, T43E, T68H, T325S, K330G, D341R, Y357G, |
| | | A360T, E391F, R395M, V405L, F436W, H455A, D490L |
| iMorphineSnFR lead 1 | Morphine | K10I, N11P, Q15G, T43I, T68H, K330G, D341R, A360T, |
| | | E391F, R395G, V405L, F436W, H455G, D490L |
| iMorphineSnFR lead 2 | Morphine | K10I, N11P, Q15G, T43I, T68H, K330G, D341R, A360T, |
| | | E391F, R395G, V405L, F436W, H455Q, D490L |
| iCodeineSnFR lead 1 | Codeine | K10I, N11F, Q15G, T43V, T68H, K330G, D341R, A360T, |
| | | E391F, R395G, V405L, F436W, H455A, D490L |
| iCodeineSnFR lead 2 | Codeine | K10I, N11L, Q15G, T43V, T68H, K330G, D341R, A360T, |
| | | E391F, R395G, V405L, F436W, H455A, D490L |
| iCodeineSnFR lead 3 | Codeine | K10I, N11P, Q15G, T43V, T68H, K330G, D341R, A360T, |
| | | E391F, R395G, V405L, F436W, H455A, D490L |
| iHydromorphoneSnFR/ | Hydrocodone | K10I, N11D, Q15G, T43V, T68H, K330G, D341R, A360T, |
| iHydrocodone lead 1 | and | E391F, R395G, V405L, F436W, H455A, D490L |
| | Hydromorphone | |
| iNaltrexoneSnFR lead 1 | Naltrexone | K10V, N11E, Q15G, T43E, T325S, K330G, D341R, A360T, |
| | | E391F, R395G, V405L, F436W, H455A, D490L |
| iNaltrexoneSnFR lead 2 | Naltrexone | K10A, N11E, Q15G, T43E, T325S, K330G, D341R, A360T, |
| | | E391F, R395G, V405L, F436W, H455A, D490L |
| iNaltrexoneSnFR lead 3 | Naltrexone | K10M, N11E, Q15G, T43E, T325S, K330G, D341R, A360T, |
| | | E391F, R395G, V405L, F436W, H455A, D490L |
| iFentanylSnFR2.0- | Fentanyl | K10A, Q15G, T43G, T68H, T325S, K330G, D341R, Y357G, |
| mTurquoise0.1 | | A360T, E391F, R395A, V405L, F436W, H455A, D490G |
| iTapentadolSnFR1.0- | Tapentadol | K10I, N11E, Q15G, T43E, T68H, T325S, K330G, D341R, |
| mTurquoise0.1 | | A360T, E391F, R395G, V405L, F436A, H455A, D490L |
| iS-methadoneSnFR1.0- | S-methadone | K10I, N11V, Q15G, T43E, T68H, T325S, K330G, D341R, |
| mTurquoise0.0 | | A360T, E391F, R395G, V405L, H455A, D490G |

The substitution mutations described herein can shift the biosensor selectivity from one opioid ligand to another opioid or non-opioid ligand or vice versa. In some embodiments, the substitution mutation in an opioid biosensor can substantially alter (e.g., decrease or increase by more than 40%, 50%, 60%, 70%, 80%, 90% or greater or decrease or increase by about, at least, or at least about 1.5, 2, 3, 4, 5, 6, 7, 8, 9, 10, 15, 20, 30, 40, 50, 100, 150, or 200-fold) the binding affinity between the biosensor and an opioid ligand. The substitution mutation can increase the binding affinity between the biosensor and one opioid ligand and decrease the binding affinity between the biosensor and another opioid or non-opioid ligand.

In some embodiments, the one or more substitution mutations (e.g., N11V) can substantially increase the binding affinity between the biosensor and S-methadone (e.g., by about, at least, or at least about 0.5, 2, 3, 4, 5, 6, 7, 8, 9, 10, 15, 20, 30, 40, 50, 100, 150, or 200-fold) while decreasing the binding affinity (e.g., by about, at least, or at least about 0.5, 2, 3, 4, 5, 6, 7, 8, 9, 10, 15, 20, 30, 40, 50, 100, 150, or 200-fold) between the biosensor and other clinically used opioids (e.g., R-methadone, fentanyl, morphine, naltrexone, rac-Tramadol, BRL 52537, met-enkephalin and EDDP) and endogenous neurotransmitters (e.g., ACh, choline, ATP, dopamine, GABA, glutamate, glycine, histamine, norepinephrine, serotonin) (see e.g., Example 5).

In some embodiments described herein, an opioid biosensor can provide enhanced selectivity in favor of one or more specific opioids and against other opioid ligands, endogenous and exogenous molecules (e.g., endogenous neurotransmitters and exogenous drugs). In some embodiments, the biosensor can have a binding affinity for a specific opioid about, at least or at least about 0.5, 2, 3, 4, 5, 6, 7, 8, 9, 10, 15, 20, 30, 40, 50, 100, 150, or 200-fold greater than for other opioids and endogenous or exogenous molecules such as endogenous neurotransmitters and exogenous drugs. In some embodiments, no detectable signal is observed for other opioids and endogenous or exogenous molecules in their physiologically relevant concentrations. Table 3 provides a list of exemplary opioid biosensors and their preferred opioid ligand.

TABLE 3
Exemplary opioid biosensors and their preferred opioid ligand.

| | | Biosensor |
|---|---|---|
| Opioid ligand | Biosensor | SEQ ID NO: |
| BRL52537 | iBRL52537SnFR lead 1 | 006 |
| BRL52537 | iBRL52537SnFR lead 2 | 007 |
| BRL52537 | iBRL52537SnFR lead 3 | 008 |
| Butorphanol | iButorphanolSnFR lead 1 | 011 |
| Codeine | iCodeineSnFR lead 1 | 016 |
| Codeine | iCodeineSnFR lead 2 | 017 |
| Codeine | iCodeineSnFR lead 3 | 018 |
| Fentanyl | iFentanylSnFR1.0 | 001 |
| Fentanyl | iFentanylSnFR2.0 | 002 |
| Hydrocodone | iHydrocodoneSnFR lead 1 | 019 |
| Hydromorphone | iHydromorphoneSnFR lead 1 | 019 |
| Levorphanol | iLevorphanolSnFR1.0 | 005 |
| Morphine | iMorphineSnFR lead 1 | 014 |
| Morphine | iMorphineSnFR lead 2 | 015 |
| Naltrexone | iNaltrexoneSnFR lead 1 | 020 |
| Naltrexone | iNaltrexoneSnFR lead 2 | 021 |
| Naltrexone | iNaltrexoneSnFR lead 3 | 022 |
| Norfentanyl | iNorfentanylSnFR lead 1 | 012 |
| R-methadone | iR-methadoneSnFR1.0 lead 1 | 022 |
| S-methadone | iS-methadoneSnFR1.0 | 004 |
| Sufentanil | iSufentanilSnFR lead 1 | 013 |
| Tapentadol | iTapentadolSnFR1.0 | 003 |
| Tramadol (racemic) | iTramadolSnFR lead 1 | 009 |
| Tramadol (racemic) | iTramadolSnFR lead 2 | 010 |
| Fentanyl | iFentanylSnFR-mTurquoise0.1 | 023 |
| Tapentadol | iTapentadolSnFR-mTurquois0.1 | 024 |
| S-methadone | iS-methadoneSnFR-mTurquoise0.0 | 025 |

In some embodiments, an opioid biosensor binds specifically to S-methadone and the opioid biosensor can have a substitution mutation at position 11. The substitution mutation can comprise N11V. In some embodiments, the opioid biosensor specific for S-methadone also has a Phe at position 436. In some embodiments, an opioid biosensor specific for S-methadone has a first PBP domain having an amino acid sequence having about, at least or at least about 850%, 900%, 9500, or a number between any two of the values, sequence identity to positions 1-75 of SEQ TD NO: 29 or SEQ ID NO: 4 or SEQ TD NO: 25 (or any of SEQ ID NOs: 1-27), a second PBP domain having an amino acid sequence having about, at least or at least about 850%, 900%, 9500 or a number between any two of the values, sequence identity to positions 325-521 of SEQ TD NO: 29 or SEQ ID NO: 4 or SEQ ID NO: 25 (or any of SEQ TD NOs: 1-27), and the opioid biosensor specific for 5-methadone also has a Val at position 11 and Phe at position 436 with reference to any one of SEQ ID NOs: 1-27 or SEQ ID NO: 29. The opioid biosensor specific for S-methadone can further comprise one or more substitution mutations selected from the group consisting of: K10I, Q15G, T43E, T68H, T325S, K330G, D341R, Y357G, A360T, E391F, R395G, V405L, H455A, D490G or a combination thereof (see, for example, Table 2). In some embodiments, an opioid biosensor specific for S-methadone comprises a first PBP domain having an amino acid sequence of positions 1-75 of SEQ ID NO: 4 or SEQ ID NO: 25 and a second PBP domain having an amino acid sequence of positions 325-521 of SEQ ID NO: 4 or SEQ ID NO: 25.

In some embodiments, an opioid biosensor binds specifically to tapentadol and the opioid biosensor can have one or more substitution mutations at position 11 and/or 436. In some embodiments, the residue at position 11 is substituted with an acidic residue (e.g., Glu). In some embodiments, the opioid biosensor that binds specifically to Tapentadol has a cation-π null mutation. For example, the residue at position 436 can be substituted with an Ala. The substitution mutation can be F436A or W436A. In some embodiments, an opioid biosensor specific for tapentadol has a first PBP domain having an amino acid sequence having about, at least or at least about 85%, 90%, 95%, or a number between any two of the values, sequence identity to positions 1-75 of SEQ ID NO: 29 or SEQ ID NO: 3 or SEQ ID NO: 24 (or any one of SEQ ID NOs: 1-27) and a second PBP domain having an amino acid sequence having about, at least or at least about 85%, 90%, 95%, or a number between any two of the values, sequence identity to positions 325-521 of SEQ ID NO: 29 or SEQ ID NO: 3 or SEQ ID NO: 24 (or any one of SEQ ID NOs: 1-27), and the opioid biosensor specific for tapentadol also has a Glu at position 11 and Ala at position 436 with reference to any one of SEQ ID NOs: 1-27 or SEQ ID NO: 29. The opioid biosensor specific for tapentadol can also have a Leu at position 490. The opioid biosensor specific for Tapentadol can further comprise one or more substitution mutations selected from the group consisting of: K10I, Q15G, T43E, T68H, T325S, K330G, D341R, Y357G, A360T, E391F, R395G, V405L, H455A, D490L, or a combination thereof (see, for example, Table 2). In some embodiments, an opioid biosensor specific for S-methadone comprises a first PBP domain having an amino acid sequence of positions 1-75 of SEQ ID NO: 3 or SEQ ID NO: 24 and a second PBP domain having an amino acid sequence of positions 325-521 of SEQ ID NO: 3 or SEQ ID NO: 24.

In some embodiments, an opioid biosensor binds specifically to fentanyl and the opioid biosensor can have one or more substitution mutations at position 11 and/or 436. In some embodiments, the residue at position 11 is a Asn. In some embodiments, the opioid biosensor that binds specifically to fentanyl has a cation-π null mutation at position 357. In some embodiments, the opioid biosensor has a Gly at position 357. In some embodiments, the substitution mutation at position 436 comprises F436W. In some embodiments, an opioid biosensor specific for fentanyl has a first PBP domain having an amino acid sequence having about, at least or at least about 85%, 90%, 95%, or a number between any two of the values, sequence identity to positions 1-75 of SEQ ID NO: 29 or SEQ ID NO: 1, SEQ ID NO: 2 or SEQ ID NO: 23 (or any one of SEQ ID NOs: 1-27) and a second PBP domain having an amino acid sequence having about, at least or at least about 85%, 90%, 95%, or a number between any two of the values, sequence identity to positions 325-521 of SEQ ID NO: 29 or SEQ ID NO: 1, SEQ ID NO: 2 or SEQ ID NO: 23 (or any one of SEQ ID NOs: 1-27), and the opioid biosensor specific for fentanyl also has an Asn at position 11 and Trp at position 436 with reference to any one of SEQ ID NOs: 1-27 or SEQ ID NO: 29. The opioid biosensor specific for fentanyl can further comprise one or more substitution mutations selected from the group consisting of: K10A, Q15G, T43G, T68H, T325S, K330G, D341R, Y357G, A360T, E391F, R395A, V405L, H455A, D490G, D490L, or a combination thereof (see, for example, Table 2). In some embodiments, an opioid biosensor specific for S-methadone comprises a first PBP domain having an amino acid sequence of positions 1-75 of SEQ ID NO: 1, SEQ ID NO: 2 or SEQ ID NO: 23 and a second PBP domain having an amino acid sequence of positions 325-521 of SEQ ID NO: 1, SEQ ID NO: 2 or SEQ ID NO: 23.

In some embodiments, an opioid biosensor binds specifically to levorphanol and the opioid biosensor can have one or more substitution mutations at position 11, 436 or 455. In some embodiments, the residue at position 11 is mutated to Pro. The substitution mutation can comprise N11P. In some embodiments, the substitution mutation at position 436 comprises F436W. In some embodiments, the residue at position 455 is Trp. The substitution mutation at position 455 can be H455W. In some embodiments, an opioid biosensor specific for levorphanol has a first PBP domain having an amino acid sequence having about, at least or at least about 85%, 90%, 95%, or a number between any two of the values, sequence identity to positions 1-75 of SEQ ID NO: 29 or SEQ ID NO: 5 (or any one of SEQ ID NOs: 1-27) and a second PBP domain having an amino acid sequence having about, at least or at least about 85%, 90%, 95%, or a number between any two of the values, sequence identity to positions 325-521 of SEQ ID NO: 29 or SEQ ID NO: 5 (or any one of SEQ ID NOs: 1-27), and the opioid biosensor specific for levorphanol also has a Pro at position 11 and Trp at position 436 with reference to any one of SEQ ID NOs: 1-27 or SEQ ID NO: 29. The opioid biosensor specific for Fentanyl can also have a Trp at position 455. The opioid biosensor specific for levorphanol can further comprise one or more substitution mutations selected from the group consisting of: K10I, Q15G, T43V, T68H, K330G, D341R, Y357G, A360T, E391F, R395G, V405L, D490L, or a combination thereof (see, for example, Table 2). In some embodiments, an opioid biosensor specific for S-methadone comprises a first PBP domain having an amino acid sequence of positions 1-75 of SEQ ID NO: 5 and a second PBP domain having an amino acid sequence of positions 325-521 of SEQ ID NO: 5.

In some embodiments, the biosensor comprises or consists of an amino acid sequence having, or having about, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to an amino acid sequence selected from the group consisting of SEQ ID NOs: 1-25. In some embodiments, the biosensor comprises, or consists of, an amino acid sequence selected from the group consisting of SEQ ID NOs: 1-25. In some embodiments, the biosensor comprises or consists of an amino acid sequence having, or having about, or having at least, or having at least about, or having at most, or having at most about, 80%, 81%, 82%, 83%, 84%, 85%, 86%, 87%, 88%, 89%, 90%, 91%, 92%, 93%, 94%, 95%, 96%, 98% or a range between any two of these values, sequence identity to an amino acid sequence selected from the group consisting of SEQ ID NOs: 26-27. In some embodiments, the biosensor comprises or consists of an amino acid sequence having, or having about, or having at least, or having at least about, or having at most, or having at most about, 80%, 81%, 82%, 83%, 84%, 85%, 86%, 87%, 88%, 89%, 90%, 91%, 92%, 93%, 94%, 95%, 96%, 98% or a range between any two of these values, sequence identity to the amino acid sequence of SEQ ID NO: 29.

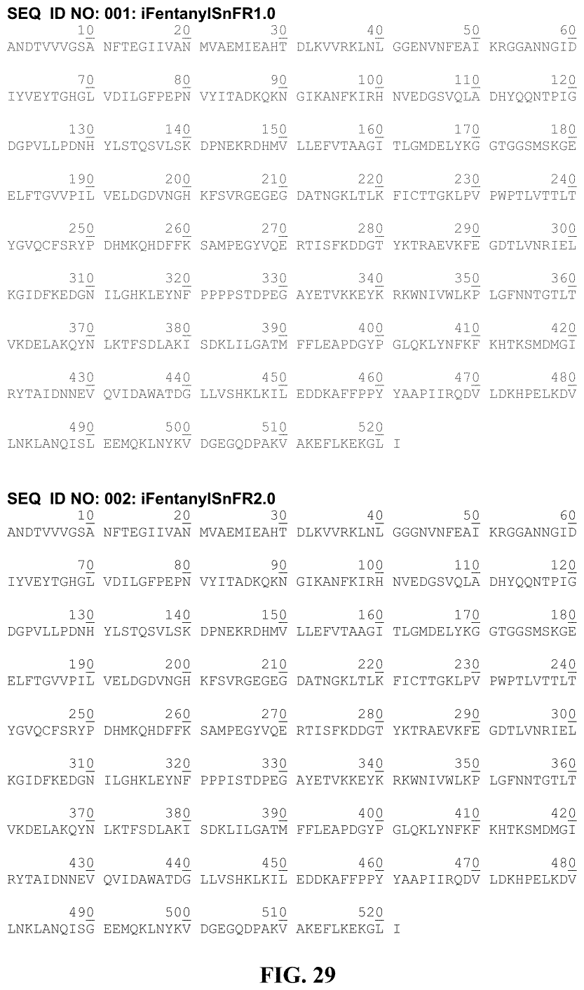

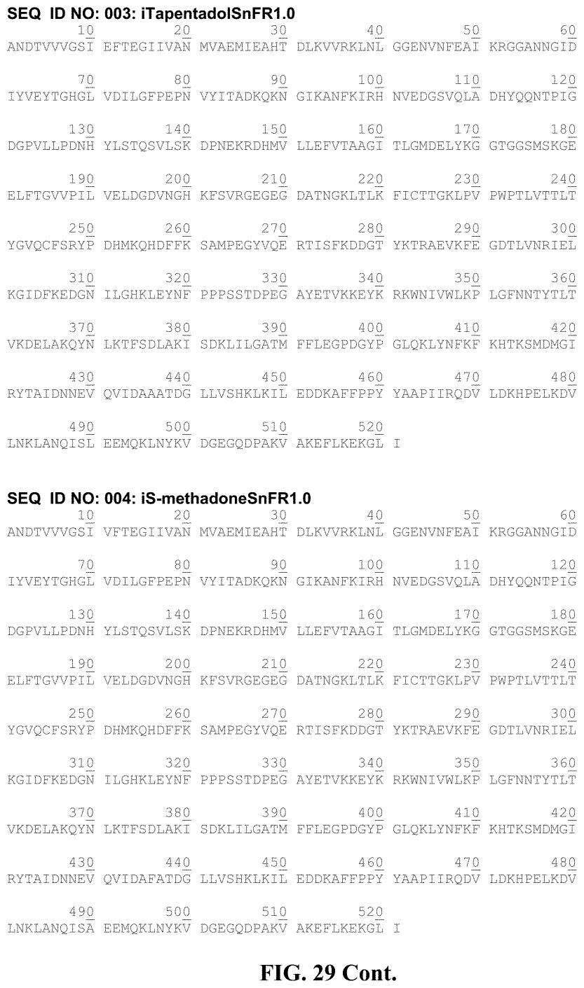

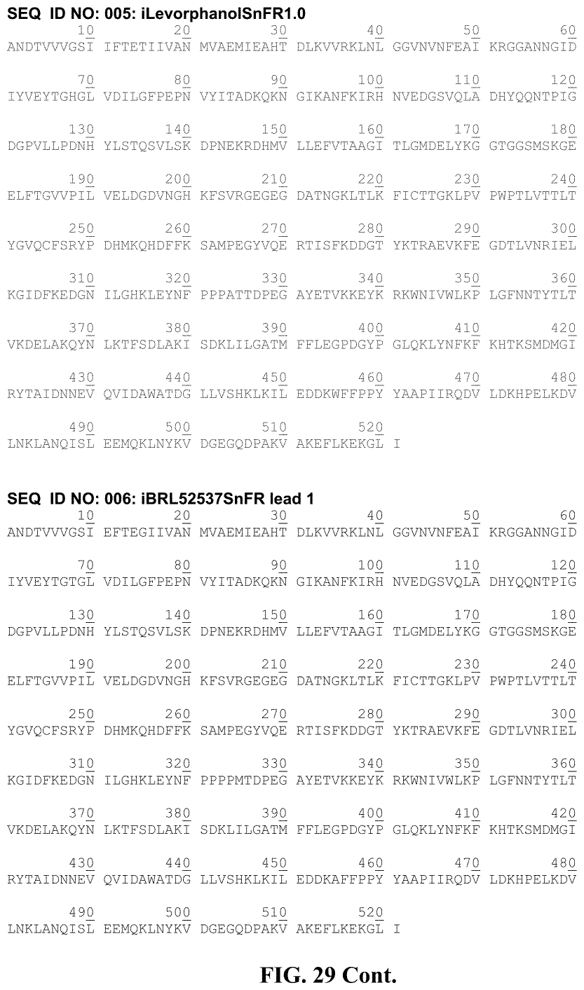

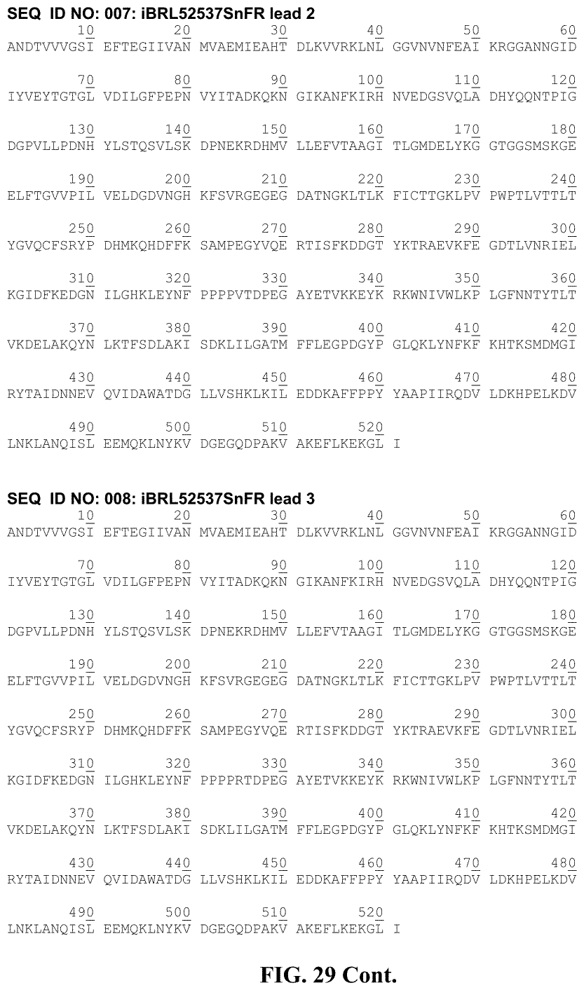

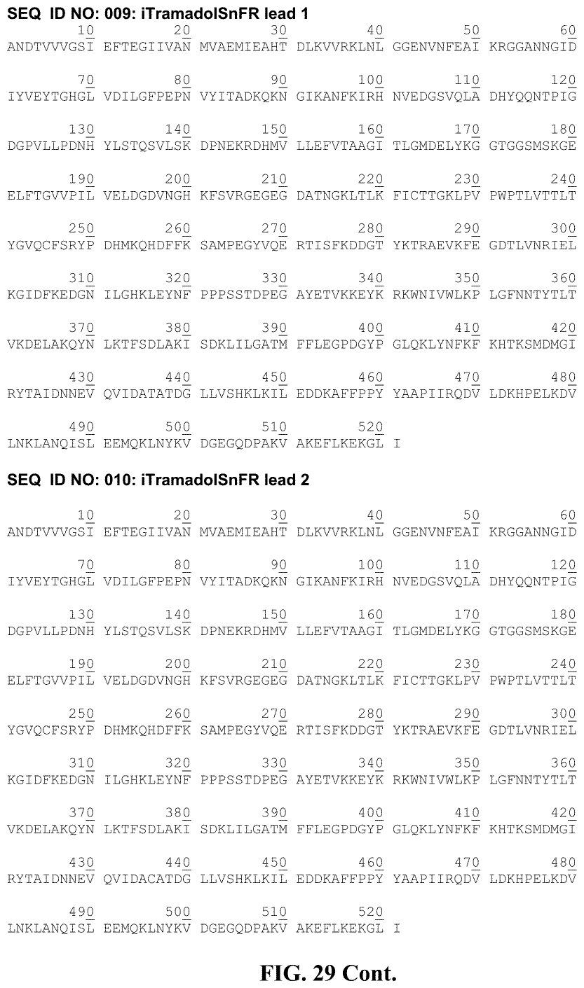

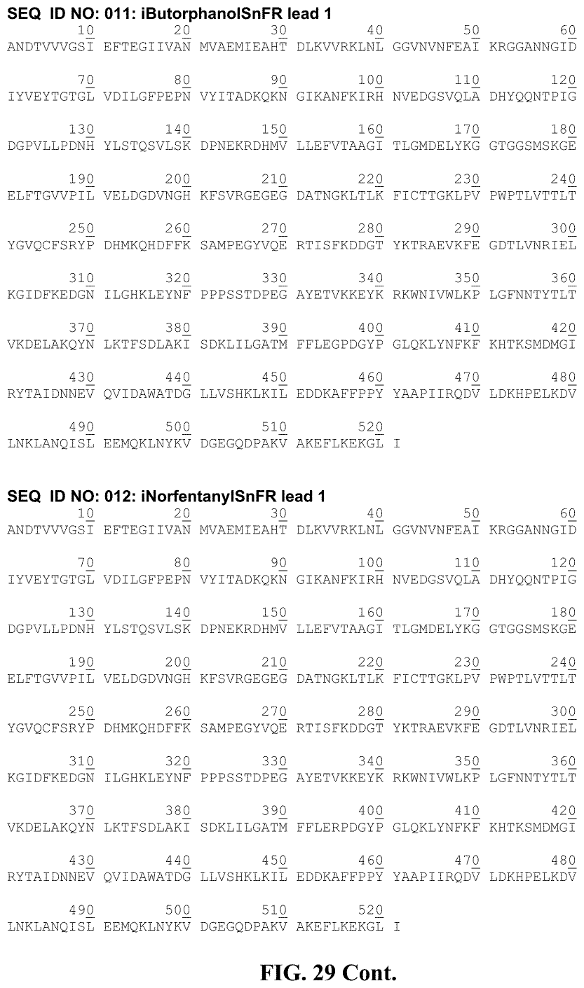

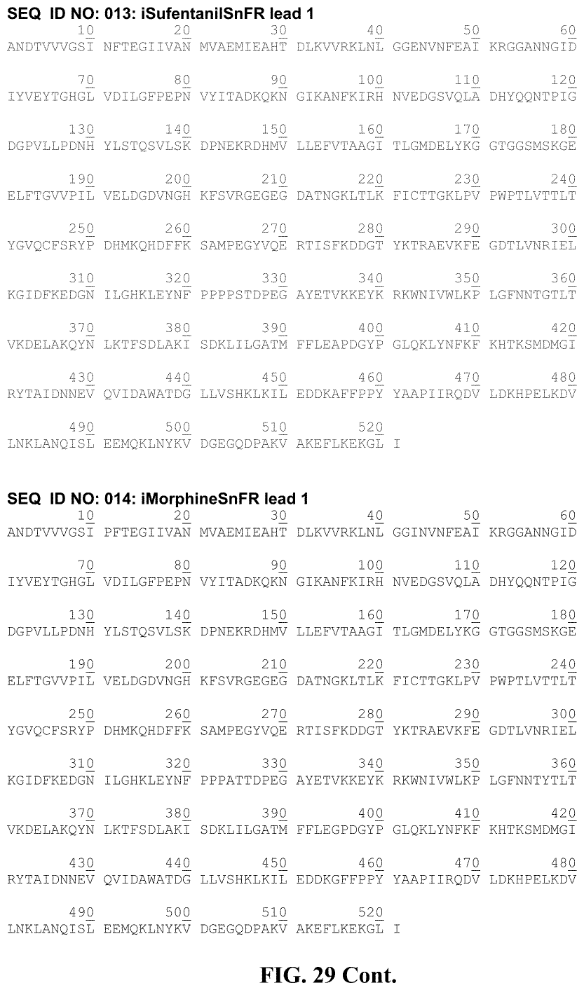

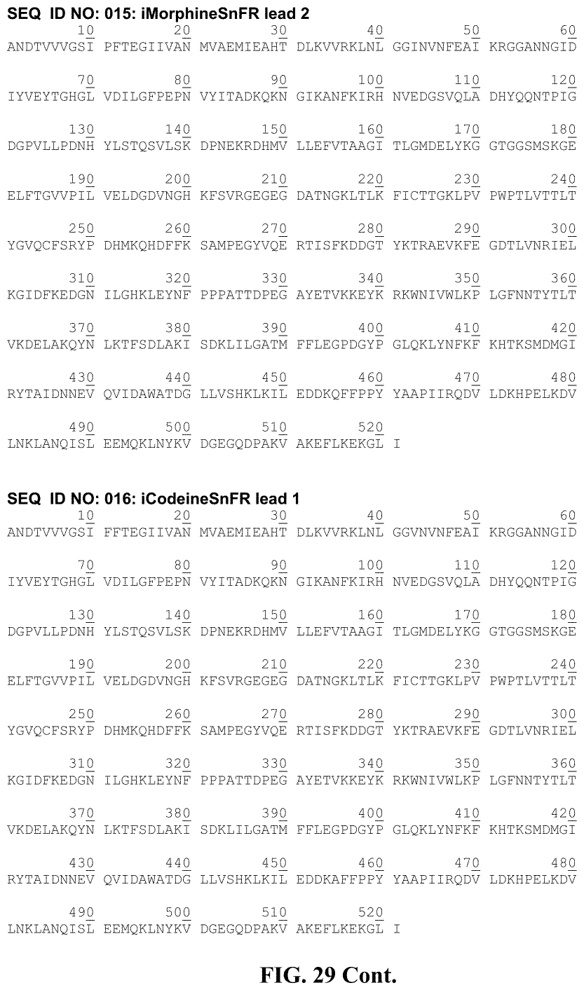

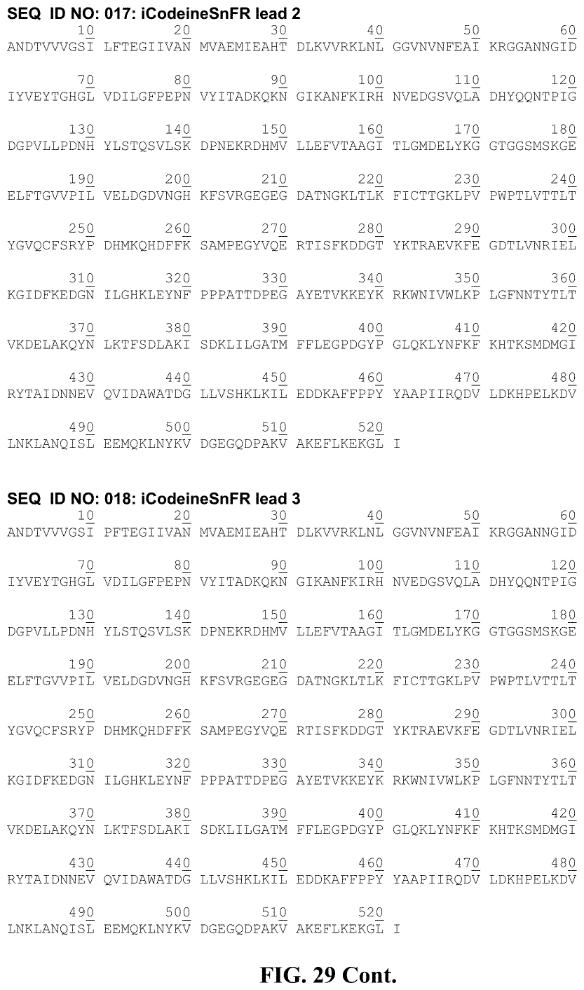

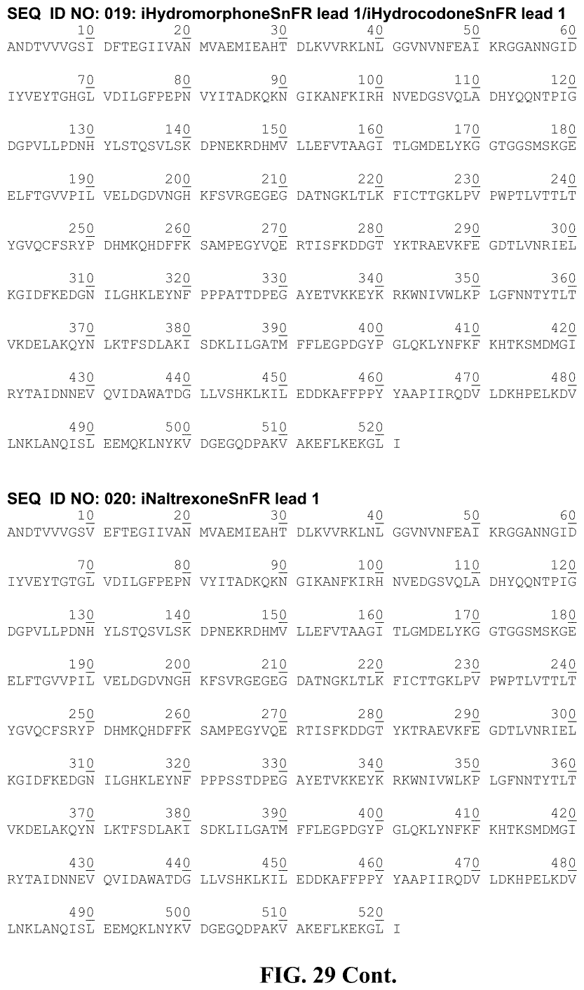

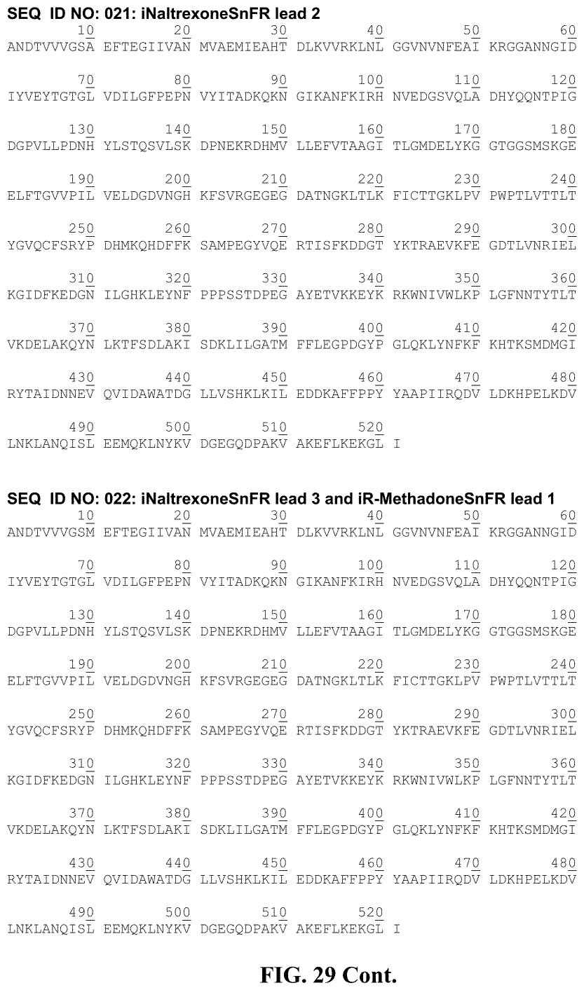

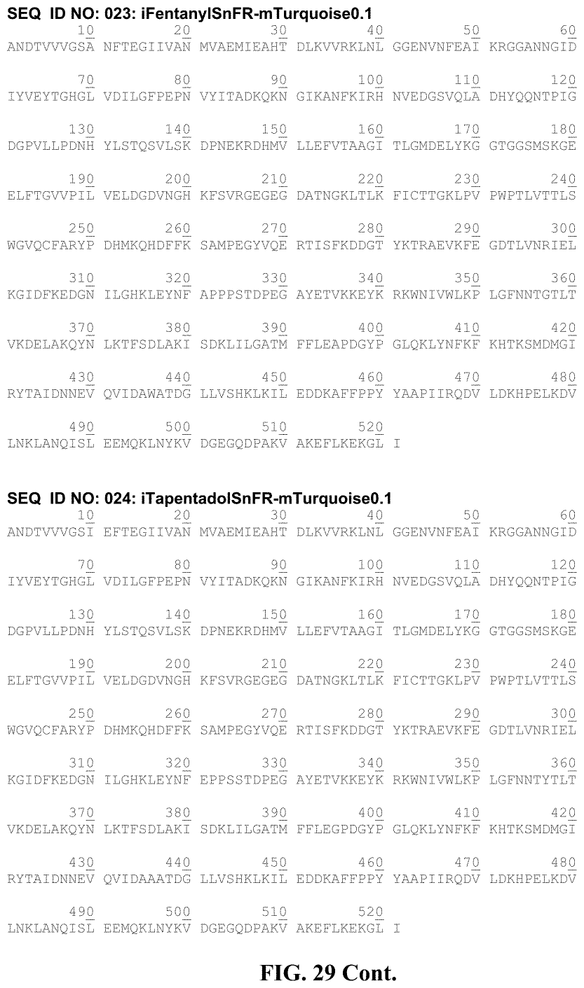

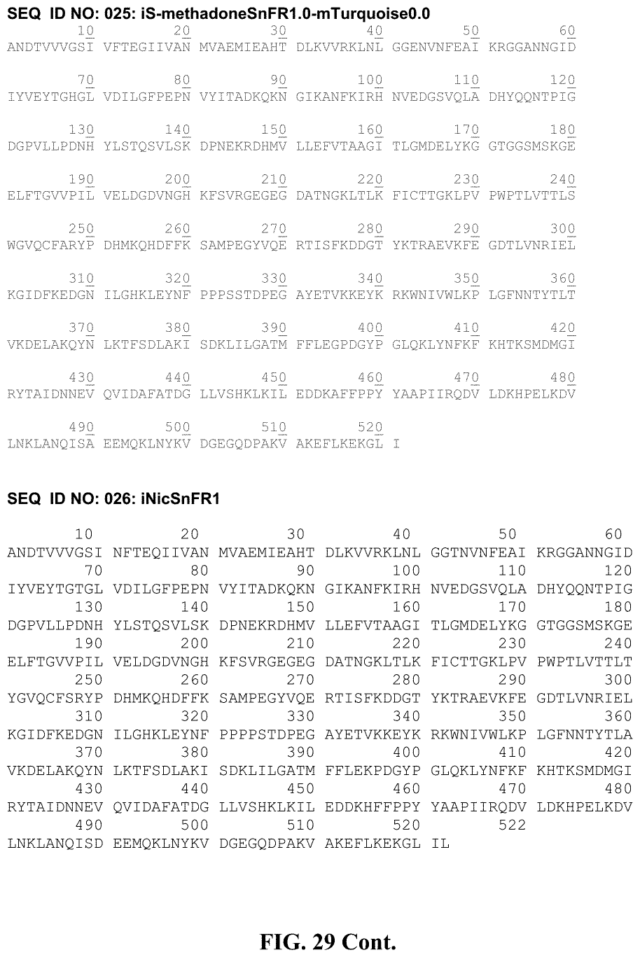

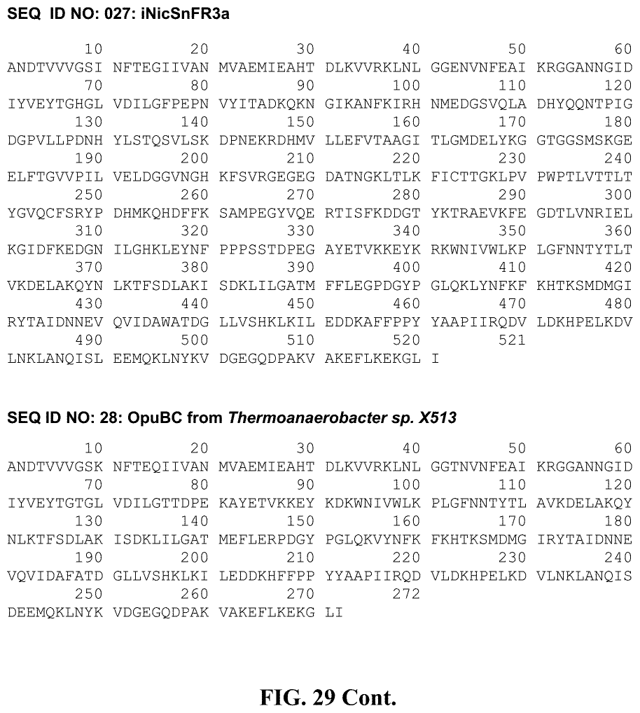

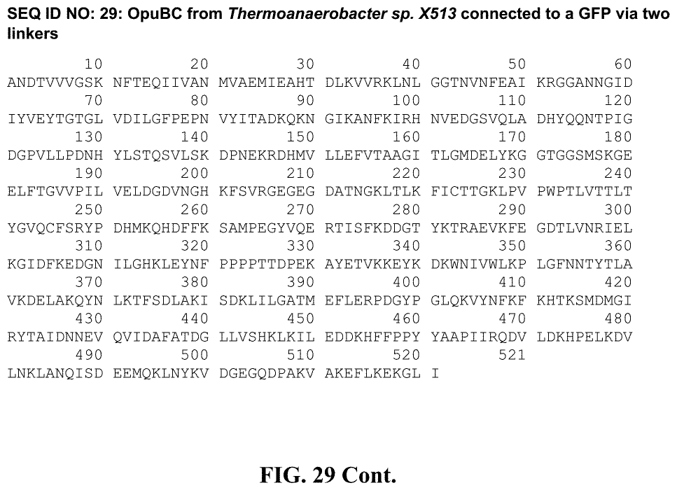

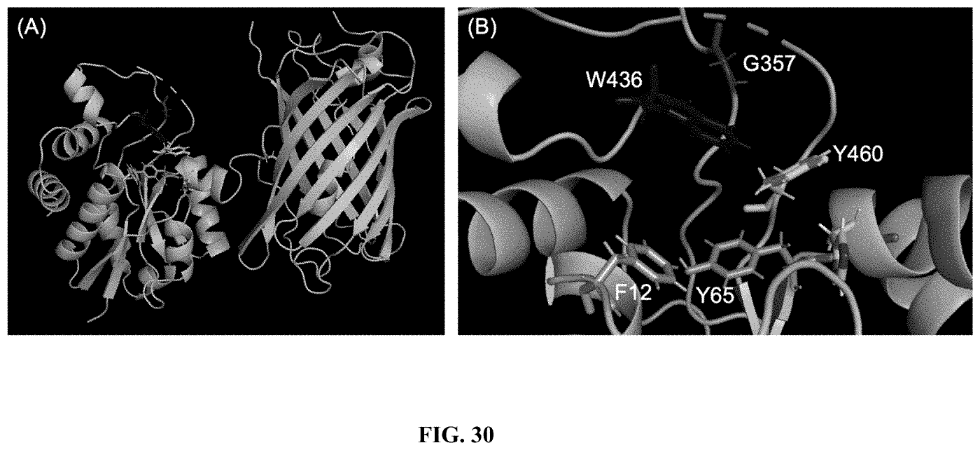

A list of non-limiting exemplary biosensor sequences are provided in FIG. 29. Positions 1-75 of a biosensor sequence corresponds to the first PBP domain (e.g., a PBP domain at the N-terminal). Positions 325-521 or 522 of a biosensor sequence corresponds to the second PBP domain (e.g., a PBP domain at the C-terminal). In some embodiments, positions 76-79 correspond to a first linker, positions 321-324 correspond to a second linker, and positions 79-320 correspond to a reporter moiety (e.g. GFP). In some embodiments, the biosensor herein described can have a construct from the N-terminal to the C-terminal: a first PBP domain—a first linker—a reporter—a second linker—a second PBP domain (see, for example, FIG. 1B).

In some embodiments, the biosensor herein described can further comprise one or more peptide tags. The term “peptide tag” as used herein means tags comprising peptide sequences introduced onto a recombinant protein. A peptide tag can be between 5 amino acids to about 50 amino acids in length. Tags can be attached to proteins for various purposes. Affinity tags are appended to proteins so that they can be purified from their crude biological source using an affinity technique. Exemplary affinity tags include chitin binding protein (CBP) and the poly(His) tag. The poly(His) tag is a widely-used protein tag; it binds to metal matrices. Chromatography tags can be used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, HA-tag and NE-tag. These tags can be used for western blotting, immunofluorescence and immunoprecipitation experiments as well as in antibody purification. Protein tags can allow specific enzymatic modification (such as biotinylation by biotin ligase) or chemical modification (such as reaction with FlAsH-EDT2 for fluorescence imaging). Tags can be combined, in order to connect proteins to multiple other components. Tags can be removable by chemical agents or by enzymatic means, such as proteolysis or splicing. For example, after purification, tags can be removed by specific proteolysis (e.g. by TEV protease, Thrombin, Factor Xa or Enteropeptidase).

### Reporter

The biosensors herein described can comprise one or more reporter such that the biosensor can trigger a detectable signal from the one or more reporter when the biosensor undergoes a conformation change upon binding an opioid ligand. The reporter can include a molecule (e.g., peptides or proteins) that can be allosterically induced to emit a detectable signal upon a conformational change. The reporter can also be a redox reporter capable of trigging an electrochemical signal.

In some embodiments, the detectable signal is detectably distinct between a signal emitted by the reporter prior to the conformational change (e.g., when the biosensor is in unbound state) and a signal emitted by the reporter moiety following the conformation change (e.g., when the biosensor is in bound state).

The reporter can be a fluorescent protein, an enzyme, a receptor and/or a transcription factor. In some embodiments, the reporter is a fluorescent protein such as a molecule containing a functional group capable of absorbing energy of a specific wavelength and emitting energy at a different wavelength (e.g., a fluorophore). The fluorescent protein can be a circularly permuted fluorescent protein (cpFP) in which the original N- and C-termini are fused using a peptide linker, while new termini are formed near the chromophore. Circular permutation methods are known in the art (see, e.g., Baird et al., Proc. Natl. Acad. Sci., 96:11241-11246, 1999; Topell and Glockshuber, Methods in Molecular Biology, 183:31-48, 2002).

As used herein, the term “fluorophore” relates to a functional group in a molecule which can absorb energy of a specific wavelength and re-emit energy at a different (but equally specific) wavelength. Fluorophore containing molecules include fluorescent proteins. The fluorophore in green fluorescent protein (GFP) includes Ser-Tyr-Gly sequence (i.e., Ser65-dehydroTyr66-Gly67), which is post-translationally modified to a 4-(p-hydroxybenzylidene)-imidazolidin-5. Exemplary fluorescent proteins include, but are not limited to, fluorescent proteins from coelenterate marine organisms, e.g., Aequorea victoria, Trachyphyllia geoffroyi, coral of the Discosoma genus, Rennilla mulleri, Anemonia sulcata, Heteractis crispa, Entacmaea quadricolor, and/or GFP including wild type and variants (e.g., EGFP, superfolder GFP), cyan fluorescent protein (CFP) and variants including mTurquoise, Cerulean, mCerulean3 (see, for example, Markwardt et al., PLoS ONE, 6(3) e17896.doi:10.1371/journal.pone.0017896), CGFP (CFP with Thr203Tyr), yellow fluorescent protein (YFP, GFP-Ser65Gly/Ser72Ala/Thr203Tyr; YFP (e.g., GFP-Ser65Gly/Ser72Ala/Thr203Tyr) with Val68Leu/Gln69Lys); Citrine (i.e., YFP-Val68Leu/Gln69Met), Venus (i.e., YFP-Phe46Leu/Phe64Leu/Met153Thr/Val 163Ala/Ser175Gly), PA-GFP (i.e., GFP-Val/163Ala/Thr203His), Kaede), red fluorescent protein (RFP, e.g., long wavelength fluorescent protein, e.g., DsRed including DsRed1, DsRed2, DsRed-Express, mRFP1, drFP583, dsFP593, asFP595), eqFP611, and/or other fluorescent proteins known in the art (see, e.g., Zhang et al., Nature Reviews, Molecular and Cellular Biology, 3:906-908, 2002). Additional fluorescent proteins and their sequences can be found, for example, at www.fpbase.org. In some embodiments, the fluorescent protein comprises a TYG (Thr-Tyr-Gly) chromophore.

In some embodiments, the reporter comprises a green fluorescent protein (GFP) or a variant thereof. In some embodiments, the GFP is a superfolder GFP. The GFP reporter can comprise or consist of an amino acid sequence having, or having about, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to positions 79-320 of a biosensor sequence selected from SEQ ID NOs: 1-27. In some embodiments, a GFP reporter comprises positions 79-320 of a biosensor sequence selected from SEQ ID NOs: 1-27.

The reporter moiety herein described can comprise additional substitution mutations. For example, the fluorescent protein can comprise a Trp-based chromophore (e.g., a SWG (Ser-Trp-Gly) chromophore) (see, for example, Example 6). In some embodiments, the Trp-based chromophore can provide a largely pH-insensitive response across the physiologically encountered pH range (e.g., across pH 4.5-8.0).

### Linkers

The biosensors herein described can include one or more genetically encoded linkers positioned between or operably linking the first PBP domain, the second PBP domain, and/or the reporter. Linker can include one or more naturally occurring or synthetic amino acids. The length of the linker can vary. For example, the linker can be about 2-15 amino acid in length. The linker can be about, at least, at least about, at most, or at most about 2, 3, 4, 5, 6, 7, 8, 9, 10, 11, 12, 13, 14, or 15 amino acids, or a range between any two of these values, in length. In some embodiments, the linker is about 4 amino acids in length. The linker can be provided between one of the PBP domains (e.g., a C-terminus of one of the PBP domains) and the other PBP domain (e.g., the N-terminus of the other PBP domain), between one of the PBP domains (e.g., the C-terminus of the first PBP domain) and a reporter (e.g., the N-terminus of the reporter), and/or between the reporter (e.g., C-terminus of the reporter) and the other PBP domain (e.g., the N-terminus of the second PBP domain). In some embodiments, the linker is rich in in proline. In some embodiments, the linker is four residues in length and rich in Proline.

The biosensor can comprise one or more linkers such as a first linker and a second linker. The first linker and the second linker do not need to be identical. In some embodiments, the first linker and the second linker are not identical. The first and the second linker can be each independently 4 amino acids in length. In some embodiments, the first and the second linker can be each independently 2, 3, 4, or 5 amino acids in length. In some embodiments, the first and the second linker can be each independently 2, 3, 4, 5, 6, 7 or 8 amino acids in length. In some embodiments, the first and the second linker can be each independently 2, 3, 4, 5, 6, 7, 8, 9, 10 or more amino acids in length. In some embodiments, the first and the second linker are identical in length.

Exemplary linkers can include, but are not limited to, positions 76-79 of any one of SEQ ID NOs: 1-27 or SEQ ID NO: 29 and positions 321-324 of any one of SEQ ID NOs: 1-27 or SEQ ID NO: 29. For example, the linker can have a sequence of FPEP, PPPS, PPPI, PPPA, PPPM, PPPV, APPP, or EPPS. In some embodiments, a biosensor disclosed herein comprises a first linker connecting the first PBP domain and the reporter and having a sequence of FPEP and a second linker connecting the reporter and the second PBP domain and having a sequence of PPPS, PPPI, PPPA, PPPM, PPPV, APPP, or EPPS.

### Nucleic Acids, Vectors and Cells

Also provided herein include nucleic acid molecules comprising the nucleotide sequences that encode one or more of the opioid biosensor molecules described herein. The nucleic acid molecule can be a recombinant expression vector, for example, a viral vector or a non-viral vector. Examples of viral vectors include, but are not limited to, adeno-associated viral vectors, lentiviral vectors, herpes simplex virus vectors, and retroviral vectors. Examples of non-viral vector include, but are not limited to, plasmids, artificial chromosomes, BACs, YACs, or PACs.

Vectors typically contain one or more regulatory elements operably linked to the nucleic acid sequences encoding the biosensors herein describe. Regulatory elements include, for example, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns as will be understood by a person skilled in the art.

Provided herein also include cells comprising the biosensors described herein (SEQ ID NOs: 1-26), including variants and/or portions of the biosensors. Cells can include, for example, eukaryotic and/or prokaryotic cells. For example, cells can include, but are not limited to, cells of E. coli, Pseudomonas, Bacillus, Streptomyces; fungi cells such as yeasts (Saccharomyces, and methylotrophic yeast such as Pichia, Candida, Hansenula, and Torulopsis), and animal cells, such as CHO, R1.1, BW, and LM cells, African Green Monkey kidney cells (for example, COS-1, COS-7, BSC-1, BSC-40), insect cells (for example, Sf9), plant cells, and human cells. Suitable human cells can include, for example, HeLa cells or human embryonic kidney (HEK) cells as well as other commercially available cells. Additional cell lines can be found, for example, at the American Type Culture Collection (ATCC).

Choice of expression vectors, the method of transformation and of making the cells described herein depend on the host system selected as will be understood by a skilled person. Transformation and transfection methods are known in the art and also described in, for example, Sambrook et al., Molecular Cloning: A Laboratory Manual (2d ed., Cold Spring Harbor Laboratory Press, Cold Spring Harbor, N.Y. (1989)) and Ausubel et al., Current Protocols in Molecular Biology, John Wiley & Sons, New York, N.Y., (1998)

The nucleic acid molecules (e.g., a vector) and/or polypeptides to cells can be delivered to cells, either in vitro or in vivo, via viral based or non-viral based delivery systems, including adenovirus vectors, adeno-associated virus (AAV) vectors, retrovirus vectors, lentiviral vectors, herpes virus vectors, liposomes, poxviruses, naked DNA administration, plasmids, cosmids, phages, encapsulated cell technology, and the like.

In some embodiments, the vector is an adeno-associated virus (AAV) vector. AAV is a replication-deficient parvovirus, the single-stranded DNA genome of which is about 4.7 kb in length including 145 nucleotides inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

AAV vectors that comprise coding regions of one or more biosensors disclosed herein are provided. The AAV vector can include a 5′ inverted terminal repeat (ITR) of AAV, a 3′ AAV ITR, a promoter, and a restriction site downstream of the promoter to allow insertion of a polynucleotide encoding one or more biosensor proteins, wherein the promoter and the restriction site are located downstream of the 5′ AAV ITR and upstream of the 3′ AAV ITR. In some embodiments, the AAV vector includes a posttranscriptional regulatory element downstream of the restriction site and upstream of the 3′ AAV ITR. In some embodiments, the AAV vectors disclosed herein can be used as AAV transfer vectors carrying a transgene encoding a biosensor protein for producing recombinant AAV viruses that can express the biosensor protein in a cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

In some embodiments, the viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in detail in US Patent Publication 2012/0232133 which is hereby incorporated by reference in its entirety.

In some embodiments, promoters can be selected for controlling transcription in mammalian host cells. Mammalian promoters can be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Expression of the biosensor molecules disclosed herein can be controlled by, for example, a cell specific promoter to allow expression only in a specific cell type.

In some embodiments, the nucleic acid molecules and/or polypeptides can be delivered via virus like particles (VLPs). VLPs comprise viral proteins derived from the structural proteins of a virus. VLPs can be naturally occurring or synthesized through the individual expression of viral structural proteins, which can then self-assemble into the virus-like structure. VLPs are generally composed of one or more viral proteins, such as particle-forming proteins referred to as capsid, coat, shell, surface and/or envelope proteins, or particle-forming polypeptides derived from these proteins.

In some embodiments, the biosensor disclosed herein can be isolated and/or purified. The terms “isolated” or “purified” refer to nucleic acid, proteins and peptides that are substantially free or free of other cellular material or culture medium when produced by recombinant techniques, or substantially free or free of precursors or other chemicals when chemically synthesized. In some embodiments, the biosensors disclosed herein are purified and lyophilized. The term “lyophilized” or “freeze-dried” refers to a dehydration process that involves freezing the product, lowering pressure and then removing the ice by sublimation as will be understood by a skilled person. Therefore, the biosensors herein disclosed can be provided as a solid material (e.g., lyophilized powder) obtained by lyophilization of an aqueous mixture. The lyophilization of the biosensors can provide increased physicochemical stability during storage and shipping compared to a liquid formulation counterpart, therefore resulting in a longer shelf life.

Also disclosed herein include transgenic animals comprising one or more cells containing the biosensors described herein. The term animal used herein refers to non-human animals, including, mammals, amphibians and birds. Examples of animals include sheep, feline, bovines, ovines, pigs, horses, rabbits, guinea pigs, mice, hamsters, rats, non-human primates, and the like. As used herein, transgenic animal refers to any animal, in which one or more of the cells of the animal contain a heterologous nucleic acid such as a heterologous nucleic acid encoding at least one of the opioid biosensor described herein. The heterologous nucleic acid can be introduced using known transgenic techniques. The nucleic acid can introduced into the cell, directly or indirectly. For example, the nucleic acid can be introduced into a precursor of the cell or by way of deliberate genetic manipulation, such as by microinjection or by infection with a recombinant virus. The nucleic acid may be integrated within a chromosome, or it may be an extrachromosomally replicating DNA. Methods for making transgenic animals are known in the art and also described in published literatures as will be understood by a person of ordinary skill in the art.

### Designing Biosensors

Provided herein also include methods to systematically design and develop an opioid biosensor herein described. The method can comprise (i) selecting a candidate biosensor protein (e.g., a PBP) comprising a binding moiety that can undergo a conformational change upon interacting with or binding to a ligand, (ii) preparing an opioid ligand panel comprising one or more target opioid herein described, (iii) screening the opioid ligand panel to identify a plurality of opioid-biosensor pairs having a signal change above a threshold value.

A signal change can be a detectable increase or decrease in the signal emitted by the reporter moiety upon interaction of the binding moiety with its respective ligand. In some embodiments, the biosensor upon binding to its respective ligand can have an increase or decrease in signal of at least or at least about 10%, 20%, 30%, 40%, 50%, 60%, 70%, 80%, 90%, 100%, 250%, 500%, 750%, 1000%, or more than 1000%, relative to the unbound biosensor. In some embodiments, the signal change is normalized using a background signal as a baseline. For example, the signal change can be detected by computing ΔF/F₀. ΔF/F₀ can be computed for each biosensor-ligand pair with the ligand at different concentrations. F₀ provides a baseline fluorescence of the biosensor with no ligand present and ΔF is defined as F_{biosensor+ligand}−F₀, i.e., the difference between the fluorescence of the biosensor bound with a ligand and the fluorescence of the unbound biosensor. As a skilled person will understand, the ΔF/F₀ will depend on the concentration of the ligand. In some embodiments, a hit is defined for a biosensor-ligand pair having ΔF/F₀ greater than 0.5 with the ligand concentration at 200 μM.

The method can further comprise (iv) optimizing the opioid-biosensor pairs to develop biosensors with enhanced sensitivity (improved ligand response) and selectivity by genetic manipulation (e.g., point mutation). The optimization can comprise identifying sites or amino acid positions in the binding moiety which are involved in the conformational change, such as the amino acid positions at the interface between the top lobe and the bottom lobe of the binding moiety (see, for example, FIG. 1B). The sites or amino acid positions suitable for optimization can be accomplished by investigating the crystal structure of biosensors in the bound and unbound states. In some instances where structure for the bound and/or unbound states are not available, analysis can be conducted by molecular modeling and/or sequence alignment to identify functionally equivalent amino acids in the binding moiety of the biosensor. Various types of mutagenesis can be used in the present disclosure to modify the biosensor to produce variants in accordance with the methods disclosed herein. Procedures that can be used include, but are not limited to, site-directed point mutagenesis, random point mutagenesis (e.g., site-saturation mutagenesis), in vitro or in vivo homologous recombination, mutagenesis using uracil containing templated, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many other known to persons of skill. In some embodiments, iterative site-saturation mutagenesis is used to modify a starting biosensor for mutation.

The method can further comprise (v) providing a reporter moiety (e.g, FP or cpFP) that, when attached to the binding moiety and upon interaction of the binding moiety with its respective ligand, can undergo a detectable conformational change. The reporter moiety may be a naturally occurring protein (e.g., FP, enzyme, receptor or transcription factor) or a variant modified to increase or decrease signal emission, to change the color of the signal, and/or to achieve increased robustness (e.g., less pH-sensitive).

The method can further comprise (vi) inserting, modifying or optimizing linker sequences between the binding moiety and the reporter moiety by genetic manipulation (e.g., point mutation) to produce biosensors with enhanced properties (e.g., improved signal-to-noise ratio). For example, the linker can be modified to increase or decrease the length. The linker can also be mutated to modulate (e.g., increase) the dynamic range of the biosensor-ligand pair. A low dynamic range can be defined as ΔFₘₐₓ/F₀<3, while a high dynamic range can be defined as ΔFₘₐₓ/F₀>3. Either the binding moiety or the reporter moiety may be circularly permuted where the circularly permuted moiety interrupts the sequence of the other protein. Linkers are introduced to connect the points of interruption. When the PBP is the moiety interrupted by the circularly permuted reporter moiety, insertion sites are selected near the PBP interdomain interface. This location in the PBP undergoes the largest conformational dynamic range and provides the greatest utility in modulating the reporter moiety. The resulting mutants are screened for baseline brightness and dynamic range upon application of a candidate ligand. Constructs with relatively low but appreciable baseline fluorescence with the greatest S-Slope are selected for linker sequence length and residue identity optimization.

The biosensor developed using the methods herein described can be cloned into a suitable vector. Biosensor expression can be placed under control of a promoter to control expression in cell types. Target amino acid sequences can be appended to the biosensor molecule to direct expression to a particular organelle or cell type. For example, some biosensors can be targeted to the endoplasmic reticulum.

In some embodiments, the method can further comprise determining the specificity and/or selectivity of the opioid biosensor with respect to one or more ligands. The sensitivity of a biosensor can be determined by calculating S-slope (e.g., (ΔF/F₀)/EC₅₀ with a Hill coefficient ˜1.0) (see e.g., Examples 1, 3, and 5). S-slope can be used for low ligand concentrations because it corresponds to the relationship between the ligand concentration and ΔF at the beginning of the dose-response relation. Details about S-Slope calculation is described in Bera K et al., Front. Cell. Neurosci., November 2019, vol. 13, doi.org/10.3389/fncel.2019.00499, the content of which is incorporated herein by reference. In some embodiments, an opioid biosensor herein described shows sensitivity for its opioid ligand when S-slope calculated for the opioid biosensor and the opioid ligand is greater than 0.1, preferably greater than 1.0. The selectivity of an opioid biosensor can be determined by calculating ratios of S-Slopes. A biosensor can be considered selective for a ligand if the S-Slope ratio is about, at least or at least ten times (e.g., 12, 15, 20, 25, 30, 500, 100 or a number between any of the two values) greater for that ligand compare to any other ligand.

A biosensor developed using the methods herein described should allow reliable and robust expression, stability, and performance across several research and diagnostic contexts. In some embodiments, protein purification can yield at least or at least about 150 nanomoles L of bacterial culture (e.g., at least or at least about 200, 220, 250 nanomoles/L of bacterial culture). Purified protein dose response can provide a comparable S-Slope to that of lysate experiments with slightly higher dynamic range (e.g., at least, at least about, at most or at most about 10%, 20%, 30%, 40%, 50%). Purified protein should maintain performance when stored as a lyophilized powder for several weeks to months at room temperature, as a solution at 4° C. for several months, or as a frozen stock at −80° C. for ˜1 year. Live cell imaging performed with Hanks' Balanced Salt Solution for Imaging (HBSS) at a suitable pH range (e.g., pH 6.7-7.8, such as pH 7.4) should reveal a greater apparent S-Slope compared to the characterization experiments conducted at pH 7.0. Stable biosensors should allow for continuous imaging under LED illumination for a certain amount of time (e.g., tens of minutes). Methods for designing and developing biosensor-opioid ligand pair are exemplified in the Examples section herein. Example 1 provides an exemplary workflow for developing opioid biosensors disclosed herein.

### Detecting Opioids Using Biosensors

Also provided herein include methods for detecting opioids using the biosensors (e.g., nucleic acids, proteins, vectors, and/or cells) disclosed herein. The method can comprise detecting a signal emitted by the reporter moiety of the opioid biosensor. For example, the method can comprise detecting a level of fluorescence emitted by a reporter moiety (e.g., FP) of the opioid biosensor. The level of fluorescence can be a signal change, i.e., a detectable increase or decrease in the signal emitted by the reporter moiety upon interaction of the binding moiety with its respective opioid ligand. In some embodiments, the signal change is considered to have an increase or decrease in signal of at least or at least about 10%, 20%, 30%, 40%, 50%, 60%, 70%, 80%, 90%, 100%, 250%, 500%, 750%, 1000%, or more than 1000%, relative to the unbound biosensor (e.g., biosensor in absence of a sample). In some embodiments, the signal change is normalized using a background signal as a baseline. For example, the signal change can be detected by computing ΔF/F₀. ΔF₀ provides a baseline fluorescence of the biosensor with no opioid ligand present and ΔF is defined as F_{biosensor}+ligand−F₀, i.e., the difference between the fluorescence of the biosensor bound with a ligand and the fluorescence of the unbound biosensor. In some embodiments, the signal emitted by the biosensor can be correlated to the absence or presence or the concentration of one or more opioid capable of interacting with or binding to the biosensor. Such correlation can be established by comparing the level of fluorescence with a level of fluorescence emitted by the biosensor in the presence of a sample comprising a known concentration or range of concentrations of the one or more opioids. In some embodiments, the level of fluorescence emitted by the biosensor in the presence (e.g., bound or bound specifically to) of a sample comprising a known concentration or range of concentrations of the one or more opioid is stored in a look-up table or on an electronic database.

In some embodiments, detecting a signal emitted by the reporter moiety of the opioid biosensor comprises obtaining a S-slope opioid dose response. The S-Slope can be defined as the (ΔFₘₐₓ/F₀)/(EC₅₀) from the fluorescence dose response as will be understood by a skilled person. Details about S-Slope calculation is described in Bera K et al., Front. Cell. Neurosci., November 2019, vol. 13, doi.org/10.3389/fncel.2019.00499, the content of which is incorporated herein by reference. In some embodiments, an opioid biosensor herein described shows sensitivity for its opioid ligand when S-slope calculated for the opioid biosensor and the opioid ligand is greater than 0.1, preferably greater than 1.0. In some specific examples, an S-Slope of ˜3 provides a detection limit of ˜10 nM and an S-Slope of ˜10 provides a detection limit ˜3 nM. The S-Slope can also be used to determine the selectivity of a biosensor. For example, the selectivity of a biosensor can be determined by calculating ratios of S-Slopes. A biosensor can be considered selective for a ligand if the S-Slope ratio is about, at least or at least ten times (e.g., 12, 15, 20, 25, 30, 500, 100 or a number between any of the two values) greater for that ligand compare to any other ligand. The S-Slopes for the biosensor disclosed herein can provide a useful detection window within the pharmacologically relevant opioid concentration range.

The opioid biosensor herein described in some embodiments can provide a robust response (e.g., a signal change) upon in contact with an opioid in a pharmaceutically relevant concentration range. The pharmaceutically relevant concentration of an opioid ligand can vary in different embodiments. In some embodiments, the pharmaceutically relevant concentration is about, at least, at least about, at most, or at most about 0.001, 0.01, 0.5, 1, 2, 3, 4, 5, 6, 7, 8, 9, 10, 15, 20, 25, 30, 40, 50, 100, 200, 300, 400, 500, 1000, 5000, 10000, 50000, 100000, 500000, 1000000 μM, or a range between any of these values. For example, an opioid biosensor that binds specifically for S-methadone (e.g., SEQ ID NO: 4 or SEQ ID NO: 25) can have sufficient selectivity against endogenous cholinergic compounds with near-zero response for ACh and choline at their physiological maximum concentrations of about 10 to 20 μM or lower. The opioid biosensor specific for S-methadone also does not detect varenicline or nicotine in their pharmacologically relevant concentration of 0 to 100 nM and ˜25 to ˜500 nM respectively (see e.g., Example 5).

Techniques for performing such methods are known in the art and/or are exemplified in more details in the Example section. For example, the method can comprise contacting one or more biosensors herein described with a target environment for a time and under conditions to detect one or more opioids and monitoring or detecting the signal emitted by the biosensor in the target environment, and correlating the signal emitted by the biosensor in the target environment with a level of the one or more opioid in the target environment. In some embodiments, contacting the one or more biosensors with a target environment allows for the expression of the one or more biosensors (e.g., when vectors comprising the nucleic acids encoding the one or more biosensors are used) in the target environment.

The target environment can comprise one or more opioids or is suspected of comprising one or more opioids. The target environment can be a cell sample, a cell line, a tissue sample, an organ, a body fluid or a biological fluid (e.g., sweat, serum, urine, blood), and/or an environment sample. In some embodiments, the target environment comprises a body fluid or biological fluid. The target environment can be in vitro, in vivo or ex vivo. Accordingly, contacting a biosensor with a target environment can be performed in vitro, in vivo or ex vivo.

In some embodiments, the target environment comprises cells. For example, the cells can be cells grown in an in vitro culture, including, primary mammalian, cells, immortalized cell lines, tumor cells, stem cells, and the like. The cells can comprise cells, tissues and organs in an ex vivo culture and cells, tissues, organs, or organs systems in vivo in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The cell can be a disease cell or a cell of disorder. Contacting the biosensor with a cell can also occur in vitro, ex vivo, or in vivo e.g. in the body of a subject.

In some embodiments, the target environment comprises a body fluid. The term “biological fluid” as used herein refers to a fluid excreted (e.g., urine, sweat), secreted (e.g., breast milk), obtained with a needle (e.g., blood or cerebrospinal fluid), or developing as a results of a pathological process (e.g., blister or cyst fluid). Additional exemplary body fluid include, but are limited to, serum, lymph, saliva, anal, and vaginal secretions, perspiration and semen of any organism.

### Kits

Also provided herein include kit of parts comprising the biosensors herein described. Kits can comprise one or more containers and the nucleic acid sequences, polypeptides, vectors, cells or combinations thereof. For example, a kit can comprise a nucleic acid sequence encoding an opioid biosensor described herein or variants or portions thereof, a polypeptide comprising an opioid biosensor described herein or variants or portions thereof, a cell comprising the nucleic acid and/or the polypeptide and/or the vector comprising the nucleic acid, or any combinations thereof. The kit can comprise reagents, buffers, administration means, and instructions for using any of the components in the kit. For example, if the kit comprises cells, the kit can also comprise cell culture medium. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

### EXAMPLES

Some aspects of the embodiments discussed above are disclosed in further detail in the following examples, which are not in any way intended to limit the scope of the present disclosure.

### Example 1

### An Exemplary Workflow for Developing Opioid Biosensors

Directed evolution was performed to convert OpuBC from a cholinergic to an opioid binding protein. This work addresses a gap in biosensor engineering: the binding motifs with large conformational changes are typically adopted from nature's bacterial periplasmic binding proteins; however, many analytes of interest are synthetic compounds. The directed evolution results suggest a general method of tuning the PBP around the cation-π box to bind other tertiary amines. This example describes an exemplary workflow for developing opioid biosensors from amine-binding periplasmic binding proteins such as OpuBC.

### 1.1 Preparing Biosensor Proteins

A biosensor library comprising a number of biosensor proteins is prepared. The biosensor library is composed of proteins fulfilling the following criteria:

1) The protein includes of a binding moiety that can undergo a conformational change and a reporter moiety that provides a direct readout of the binding event.

2) The binding moiety effects a change in the reporter moiety's state through linker sequences that have been optimized. In one instance, these linkers are four residues in length. In one instance, these linkers are mutated to increase or decrease the length.

3) The binding moiety can bind primary, secondary, or tertiary amines with molecular weight in the relevant range: 100 to at least 600 Da.

4) The fusion protein can be expressed in E. coli such as BL21 DE3 and purified to yield a stable, soluble protein.

In some embodiments, the amine-binding moiety is a periplasmic binding protein, OpuBC homologue from Thermoanaerobacter sp. X513 and the reporter moiety is a circularly permuted GFP (cpGFP). For other periplasmic binding proteins or reporter proteins, linker insertion sites should be screened to provide multiple starting points for directed evolution (Marvin '13).

### 1.2 Preparing an Opioid Ligand Panel

To prepare an opioid ligand panel, the opioid ligands are selected to represent μ, δ, and κ-opioid pharmacology and the various structural subclasses of these opioids. The following mu opioid subclasses are sampled: (1) morphine and its analogs, (2) morphinan, (3) fentanyl and its analogs, (4) other phenyl piperidines and their analogs, (5) benzenoid monoamine, and (6) positive/negative/silent allosteric modulators. Selected κ-opioids sample comprise both natural products and synthetic ligands. These synthetic ligands cover both selective and non-selective kappa opioids. δ-opioids, fewer in number, are selected for prominence in the research literature.

3×PBS pH 7.0 is generally used to prepare 2 mM stock solutions of each drug. 3×PBS pH 7.0 provides reliable buffering in the screening experiments and does not interfere with the biosensor's function. These drugs are solubilized in aqueous buffer (with no organic solvent) if possible, to avoid use of organic solvents. The minimal v/v fraction of DMSO in PBS needed to prepare a 2 mM stock solution is unknown for many opioids, especially research drugs in the κ and δ-opioid classes. For compounds known to have low water solubility, ˜1 mg is used for a solubility test. First, a volume of PBS is added followed by an equal volume of DMSO. If the material is fully solvated, then more PBS is added; if the material is still not fully solvated, more DMSO is added. The process is repeated until the volume added reaches that needed for a 2 mM solution. 0, 25, 50, 75, or 100% DMSO was used to solvate δ and κ-opioids.

### 1.3 Discovering “Opioid x Biosensor” Hits

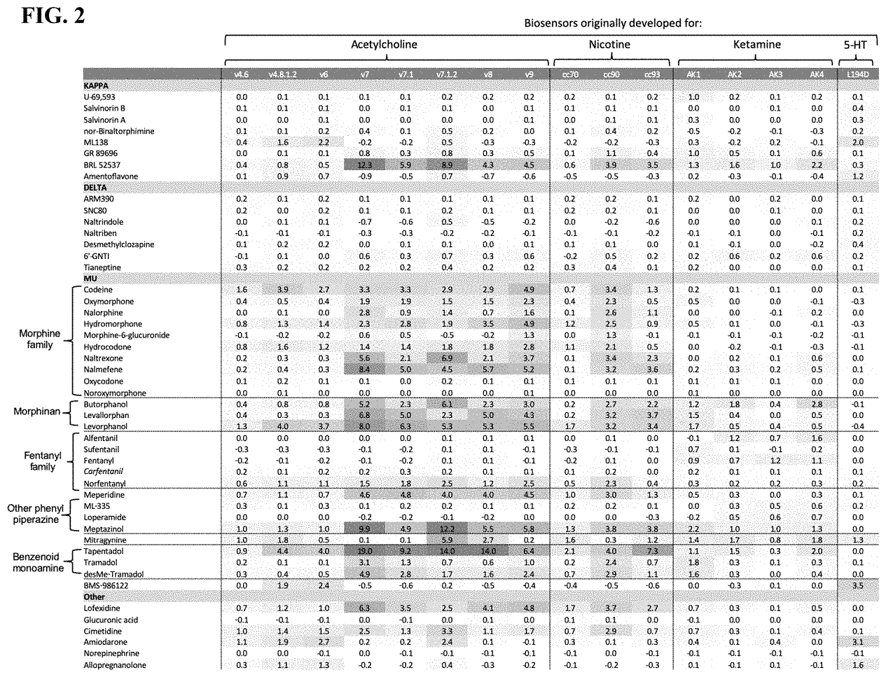

A “opioid x biosensor” hit is defined for a biosensor-opioid pair with ΔF/F₀>0.5 at 200 μM. This criterion filters out biosensors that will not likely be evolvable in ˜5 rounds of site-saturation mutagenesis (SSM) to yield the desired affinity and dynamic range. Example 2 below provides an example an “opioid x biosensor” screen (FIG. 2). These biosensor-opioid pairs are then validated in dose responses across ˜10 nM to 200 μM opioid concentration. The best hits in terms of dynamic range and/or apparent affinity provide starting points for directed evolution.

### 1.4 Structure-Function Relationships for OpuBC and Opioids

The best hits for different opioid classes can be clustered into the following groups (see Examples 2-4 for details): Fentanyl and its analogs but not it's metabolite: AK1-4; Morphine and its analogs: v9; Morphinan ligands: v7 and v9; μ-opioid antagonists: v7 and v7.1.2; Small (<300 Da) opioids: v7, v7.1, v7.1.2; κ-opioids: v7, cc90, and AK1; δ-opioids: v7.1.2.

AK1 variants used herein comprises the following substitution mutations with respect to the wild type OpuBC (SEQ ID NO: 29): K10I, Q15G, T43E, T68H, T325S, K330G, D341R, Y357G, A360T, E391F, R395G, V405L, F436W, H455A, and D490L.

V7 variant used herein comprises the following substitution mutations with respect to the wild type OpuBC (SEQ ID NO: 29): K10I, N11E, Q15G, T43E, T68H, T325S, K330G, D341R, Y357G, A360T, E391F, R395G, V405L, F436W, H455A, and D490L.

V9 variant used herein comprises the following substitution mutations with respect to the wild type OpuBC (SEQ ID NO: 29): K10I, N11I, Q15G, T43V, T68H, K330G, D341R, Y357G, A360T, E391F, R395G, V405L, F436W, H455A, and D490L.

Categorical pairs of affinity and dynamic range for hits warrant different directed evolution strategies. For hits, “low dynamic range” is defined as ΔFₘₐₓ/F₀<3, and “high dynamic range” is otherwise. “High affinity” is defined as EC₅₀<10 μM, and “low affinity” is otherwise.

Pairs with high dynamic range and high affinity likely have sufficient sensitivity for application but lack specificity. A cation-π residue site-saturation mutagenesis (SSM) screen may be conducted to abolish cholinergic sensitivity. Examples include iTapentadolSnFR1.0 developed by a single mutation at a cation-π residue (W436A).

Pair with high dynamic range and low affinity occur most frequently with opioid-hit pairs. SSM should first focus on binding pocket and second shell residues until the desired affinity is achieved. If there is a tradeoff with dynamic range, the linker residues can be mutated at a later point.

Biosensor of low dynamic range and high affinity likely has a near-suitable binding pocket but is limited by non-optimal linkers. Site-saturation mutagenesis at the linker positions should yield a variant wither higher dynamic range. Binding pocket evolution can then be revisited as needed. Examples in this category include iFentanylSnFR1.0/2.0. A key step involves SSM at all residues in the second linker.

For pairs with low dynamic range and low affinity, 4-6 residues should be selected spanning the binding pocket, second shell, and linker residues for site-saturation mutagenesis. If no improvements are discovered, then the compounds can be revisited following section 1.6 below.

### 1.5 Evolving Sensitive and Selective Opioid Biosensor Leads

As detailed in Examples 3-4 below, iterative single position site-saturation mutagenesis is sufficient to develop sensitive and selective opioids biosensors. A “lead” is defined as a biosensor participating in a hit that is evolved for improved response and selectivity to the ligand. Generally, picking four positions for site-saturation mutagenesis yield at least one improved lead. If screens in parallel identify improvements at multiple residues, the evolutions may be performed in series to gain additive improvements.

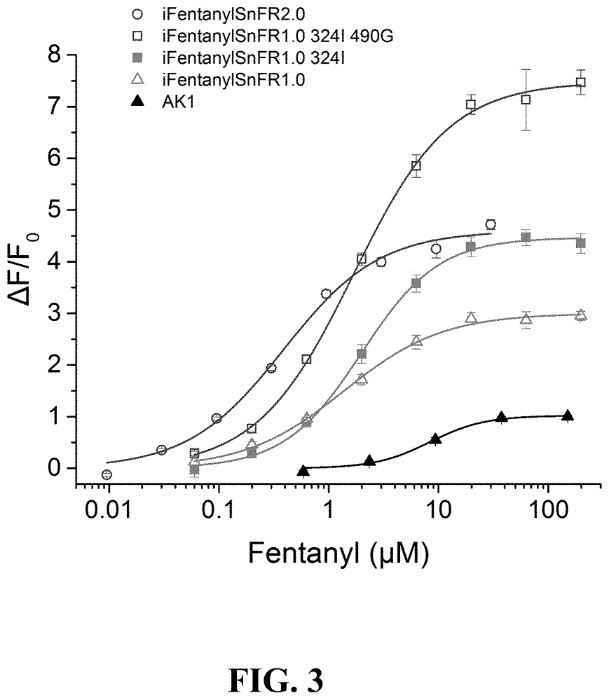

Each SSM step can screen multiple ligands using the same culture plate to either find evolution branches towards other iOpioidSnFRs or determine if a mutant confers selectivity against some ligand. FIG. 3 provides an example of the progression from a hit (fentanyl+AK1, a suboptimal biosensor) to a presently optimal iFentanylSnFR2.0.

Methadone is often another opioid detected by many hits (see Example 3 for iLevorphanolSnFR1.0 evolution) and should be considered for negative selection where the clones are picked for non-response to another ligand.

1.6 Revisiting Compounds that Did not Participate in a Hit

For some opioids, a suitable biosensor hit may not be identified, or a lead could not be evolved in one or two rounds of directed evolution. The directed evolution projects for exemplar biosensor-opioid pairs yield rich biosensor protein panels likely to yield more favorable hits compared to the initial screen. For example, no suitable hits for alfentanil and remifentanil could be found, but one of the biosensors generated along the path to iFentanylSnFR2.0 may be better optimized for these fentanyl analogs. iFentanylSnFR1.0 and iFentanylSnFR2.0 provide improved sensitivity to sufentanil compared to “AK1” (but insufficient to create selectivity problems in the pharmacological sufentanil concentration range). Subsets of the opioid panel may be periodically screened against libraries of newly generated biosensors.

### 1.7 Isothermal Titration Calorimetry

Isothermal titration calorimetry provides a measure of ligand binding affinity independent of fluorescence. The binding pocket interaction can be further probed by mutating a cation-π residue to Ala or Gly and performing ITC. If the biosensor has only one binding site, then no appreciable heats from the protein are expected.

### 1.8 Addition of Targeting Sequences and Promoters

Biosensor expression may be placed under control of a promoter to control expression in cell types. Address amino acid sequences may be appended to the biosensor protein to direct expression to an organelle. This modification has not adversely affected biosensor itself in live cell imaging to date; however, expression level must be controlled. For example, some biosensors targeted to the endoplasmic reticulum express and accumulate robustly leading to unhealthy ER and/or decreased biosensor dynamic range.

### Example 2

### Exemplary Embodiments for Discovering “Opioid x Biosensor” Hits

### “Opioid x Biosensor” Screen

This opioid x biosensor screen tested the hypothesis that OpuBC-based biosensors evolved for other ligands can detect μ, κ, and δ opioids. The response of each of the biosensors to 200 μM of each opioid is shown as ΔF/F0 in the green map (FIG. 2). Discovered hits involved nearly all μ opioids, ˜½ of κ opioids, and two δ opioids. Hits were discovered for at least one compound considered a μ, κ, or δ-selective ligand. All attempted directed evolution for hits identified in this screen have yielded leads (see Examples 3-4 below).

The need to dissolve all δ opioids in buffers with DMSO likely contributed to lower dynamic range and associated lower likelihood of observing hits. Rescreening at lower δ opioid concentration with a proportionately lower fraction DMSO may yield an increased hit frequency in this class. Moreover, biosensors constructed with other amine-binding periplasmic binding proteins may be screened against this opioid panel to discover additional hits using the same method described in Example 1.

### Individual Opioid x Biosensor Dose Response

Ligands of high interest may be immediately screened against an available biosensor library for full dose response relationships. In the examples herein described, the biosensor libraries differ by 1 to ˜10 point mutations in residues of the binding pocket, second shell, and linkers. In addition to discovering hits, this type of screen generates structure-function information regarding binding affinity and selectivity over other ligands or isomers of the same ligand.

### Example 3

### Exemplary Opioid Biosensors

Biosensors for fentanyl, S-methadone, tapentadol, and levorphanol have been evolved for sensitivity in the pharmacologically relevant drug concentration range. These sensors, iFentanylSnFR2.0, iS-methadoneSnFR1.0, iTapentadolSnFR1.0, and iLevorphanolSnFR1.0 are exemplar opioid biosensors with extensive characterization. These sensors all exhibit excellent selectivity against endogenous molecules, enabling studies in live cells and in biofluids such as sweat and serum. iFentanylSnFR2.0, iS-methadoneSnFR1.0, and iTapentadolSnFR1.0 have an additional level of selectivity: low or no response to other opioids including their metabolites. This “one sensor-one drug” feature distinguishes these biosensors from nature's opioid receptors and other synthetic proteins to date. The development of these exemplar opioid biosensors generalizes the directed evolution method to several opioid structure classes.

### Biosensor Sensitivity

FIGS. 4A-4D provide dose responses for each purified biosensor-opioid pair in solution shows exemplar dose response relations. The S-Slope is defined as the (ΔFmax/F0)/(EC50) from the fluorescence dose response (Bera '19). An S-Slope of ˜3 provides a detection limit of ˜10 nM and a ˜10 provides a detection limit ˜3 nM (HAL, unpublished). The S-Slopes for each of these exemplar biosensors provide a useful detection window within the pharmacologically relevant [opioid] range.

### Isothermal Titration Calorimetry

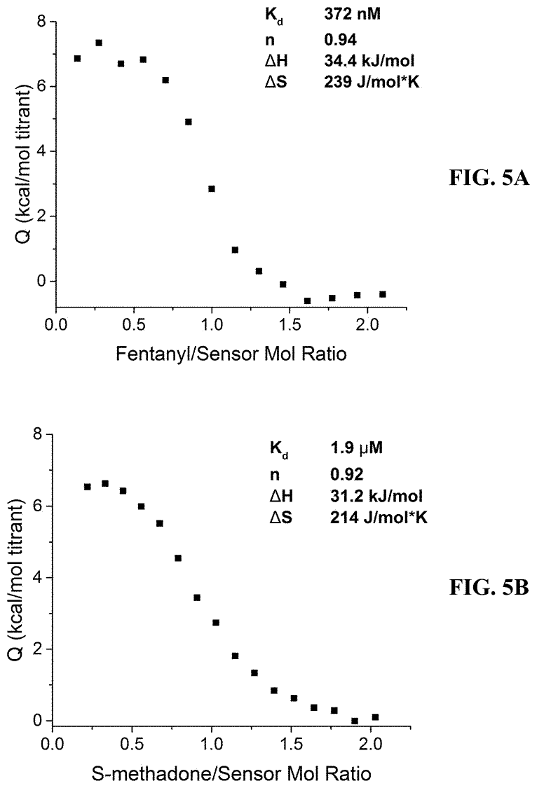

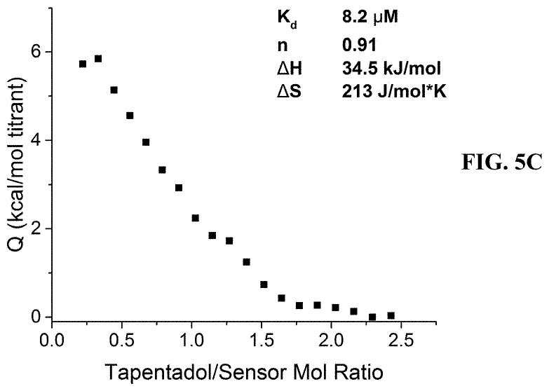

Isothermal titration calorimetry (ITC) studies the binding affinity independent of fluorescence. FIGS. 5A-5C show that ITC verifies the K_{d} nearly matches the EC₅₀ found from fluorescence dose-response for iFentanylSnFR2.0, iS-methadoneSnFR1.0, and iTapentadolSnFR1.0. In FIG. 5A, 2.5 μL of 200 μM fentanyl was injected into the cell with 20 μM iFentanylSnFR2.0. In FIG. 5B, 2.5 μL of 400 μM S-methadone was injected into the cell with 185 μL of 40 μM iS-methadoneSnFR1.0. In FIG. 5C, 2.5 μL of 450 μM tapentadol was injected into the cell with 45 μM iTapentadolSnFR1.0.

In all cases, the binding event drives an enthalpically-disfavored but entropically-favored process. Compared to iTapentadolSnFR1.0, iFentanylSnFR2.0 appears to have improved affinity through increased entropic favorability and for iS-methadoneSnFR1.0, increased enthalpic favorability. These results indicate that the directed evolution tunes interactions beyond the binding pocket itself. The three biosensor-drug change encompass a range of 20-fold in affinity and, 10% in ΔH and 10% ΔS—and this was obtained by mutating the binding pocket and linker (a proline to isoleucine in iFentanylSnFR2.0's linker).

### Selectivity Against an Endogenous Opioid Peptide

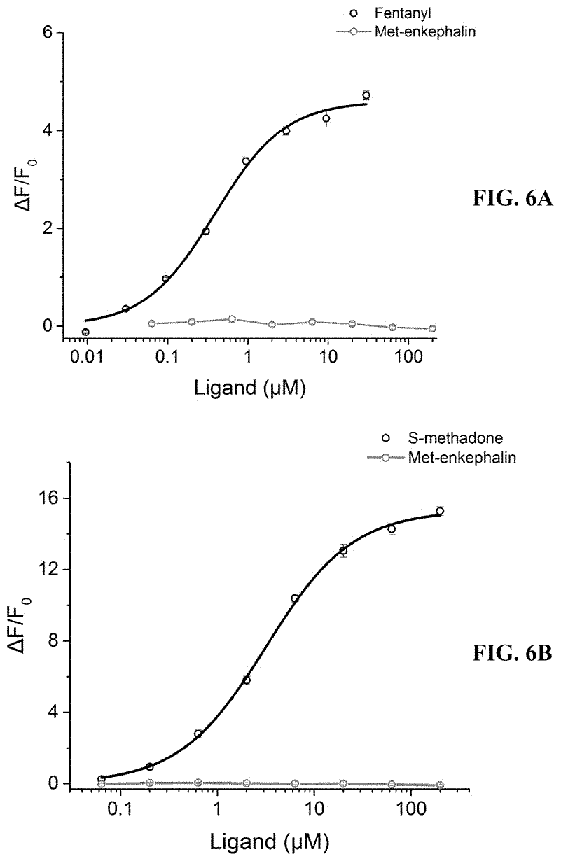

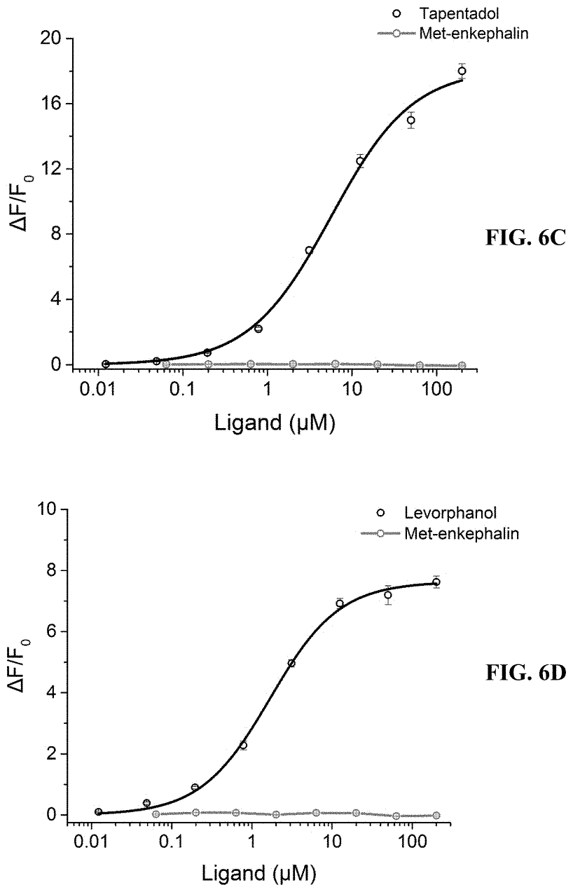

These biosensors are based on bacterial PBPs and present minimal crosstalk with mammalian biological systems. Whereas the mammalian μ-opioid receptor detects both opioid drugs and the endogenous opioid peptides, iOpioidSnFRs show little or no detectable response to an endogenous peptide, met-enkephalin (FIGS. 6A-6D). This null response is expected to generalize to other endogenous opioid peptides which are generally larger than met-enkephalin (e.g. beta-endorphin).

### Selectivity Against Neurotransmitters

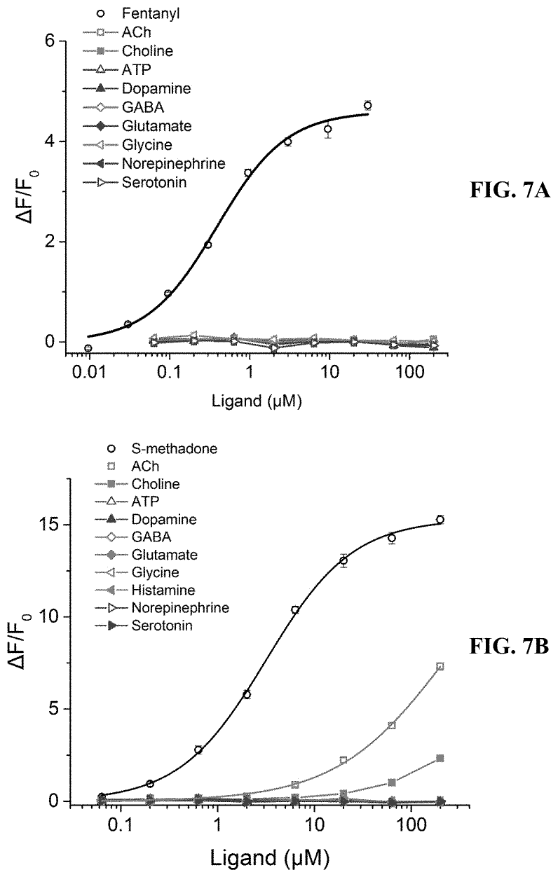

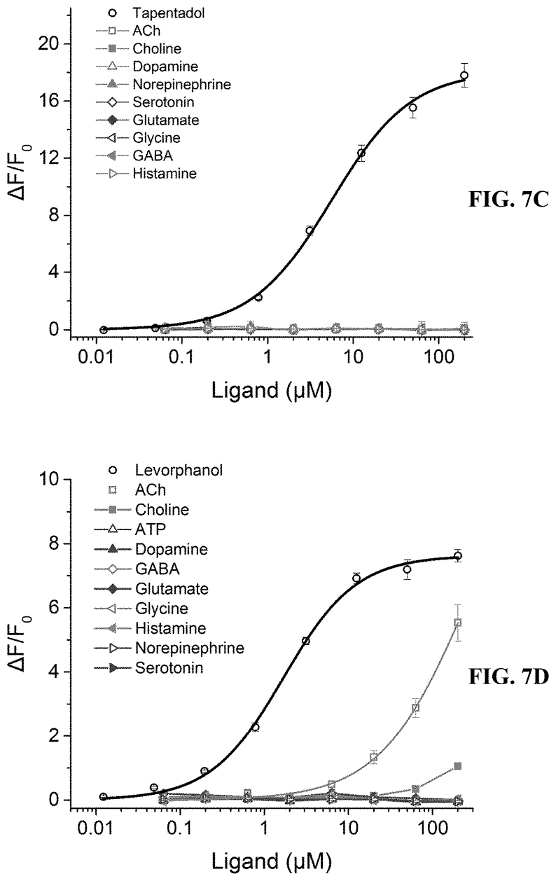

OpuBC is a choline-binding protein in nature and the biosensors used in Example 2 all respond to acetylcholine and choline at physiologically relevant concentrations of those ligands. Biosensors should not display response to the endogenous neurotransmitters to minimize confounds. FIGS. 7A-7D show the exemplar biosensors have no or minimal response to neurotransmitters. iFentanylSnFR2.0 and iTapentadolSnFR1.0 have key “cation-π null” mutations, 357G and 436A respectively, that alone render them completely insensitive to acetylcholine and choline. iS-methadoneSnFR1.0 and iLevorphanolSnFR1.0 do not tolerate those cation-π mutations and required progressive evolution away from cholinergic sensitivity. These two sensors exhibit S-Slopes of ˜0.1 and ˜0.05 for acetylcholine respectively, providing a minimal confounding signal (on the order 50 to 100-fold less than the response to the opioid).

### Selectivity Against Other Opioids

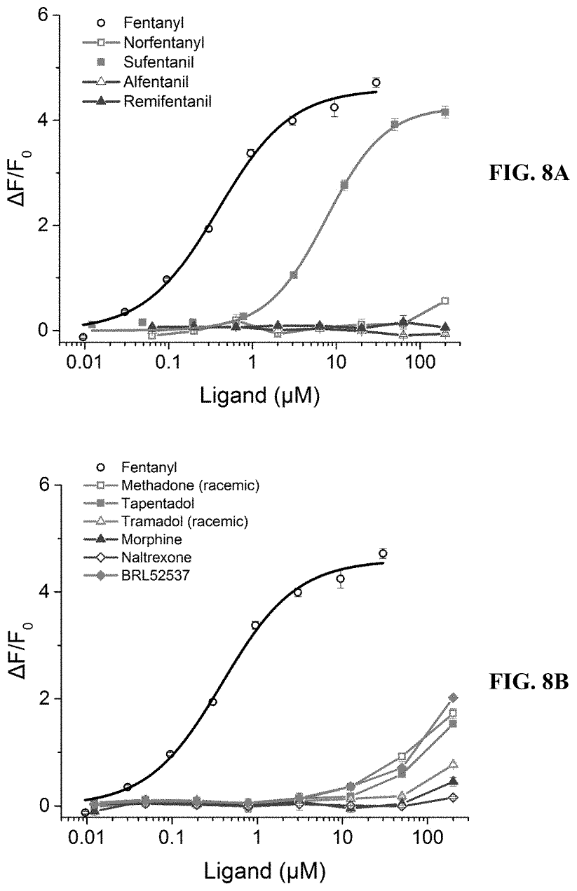

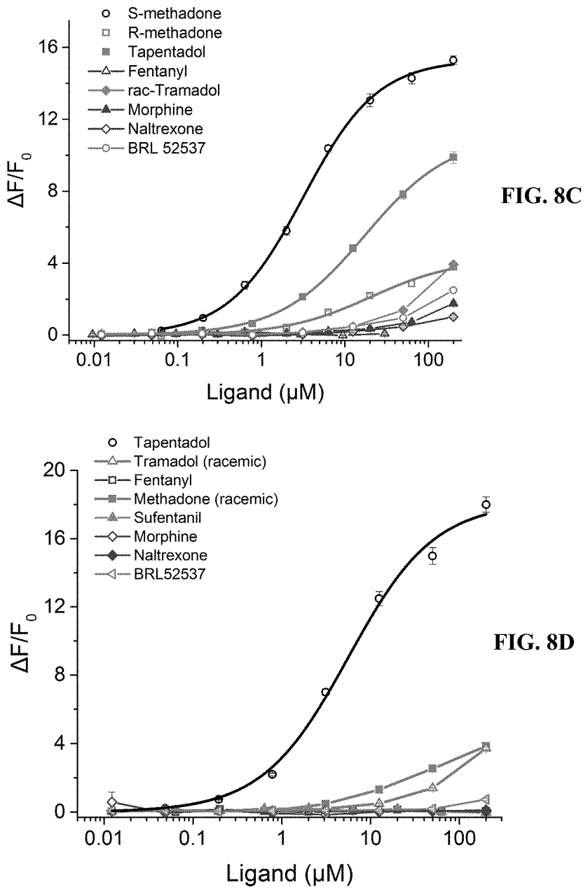

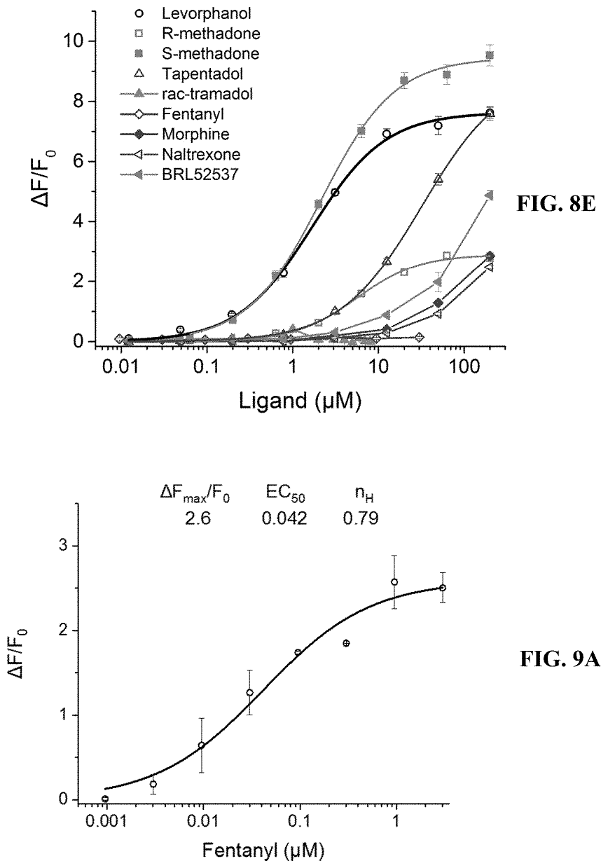

iFentanylSnFR2.0, iS-methadoneSnFR1.0, and iTapentadolSnFR1.0 provide sufficient selectivity over other opioids in their pharmacologically relevant range (FIGS. 8A-8D). iFentanylSnFR2.0 shows sufficient selectivity against other fentanyl analogs and fentanyl's major metabolite, norfentanyl. The S-Slope for sufentanil is 0.57—insufficient to detect below 100 nM (the pharmacologically and abuse-relevant range) (FIG. 8A). The S-Slope for fentanyl is ˜20× that of sufentanil. No significant response is shown for the other ligands under 100 M. iFentanylSnFR2.0 shows no significant response to several opioids under ˜3 μM (FIG. 8B). S-Slopes for responses to other opioids are <<0.1. iS-methadoneSnFR1.0 shows selectivity against the opioids shown in FIG. 8C. The S-Slope for R-methadone is ˜0.2, a ˜24-fold difference compared to that for S-methadone. The S-Slope for tapentadol is 0.57, a ˜8-fold difference compared to that for S-methadone. iTapentadolSnFR1.0 also shows selectivity against opioids shown in FIG. 8D. S-Slopes for responses other opioids are <0.1. iLevorphanolSnFR1.0 provides such selectivity except against methadone (FIG. 8E). iS-methadoneSnFR1.0 has appreciable response to tapentadol just above the high end of the pharmacologically relevant [tapentadol](several micromolar) (FIG. 8C). In these two cases, further directed evolution would be necessary for complete specificity through the low to mid-micromolar range.

Biosensors with cholinergic-null mutations at cation-π residues, iFentanylSnFR2.0 and iTapentadolSnFR1.0, demonstrate the best selectivity against other ligands.

iFentanylSnFR2.0 demonstrates selectivity not only against other opioids (FIG. 8B) but also against its own family of analogs (FIG. 8A). Critically, iFentanylSnFR2.0 is selective against norfentanyl, the major metabolite of fentanyl. Remarkably, iTapentadolSnFR1.0 is selective (S-Slope <0.1) against tramadol, the structure that provided the basis for the design of tapentadol (Raffa '12).

### Performance in Sweat, Saliva, and Blood Serum

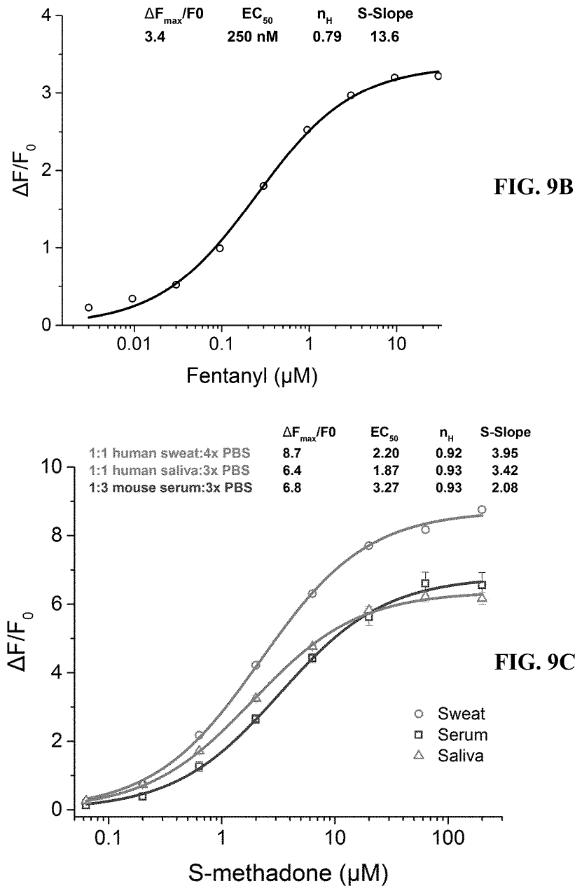

Routine clinical methods have been established for detecting opioids in blood. For fentanyl and methadone, there are examples of [opioid] in sweat providing a means for therapeutic monitoring. Here, 50% of the solution is a prepared buffer mixed with 50% of either human sweat or mouse serum (FIGS. 9A-9C). FIG. 9A shows the iFentanylSnFR2.0 dose response to fentanyl in 50% mouse serum in 6×PBS pH 7.4. The Hill fit has not been corrected for ligand depletion. FIG. 9B shows iFentanylSnFR2.0 dose response to fentanyl in 50% human sweat in 6× PBS pH 7.4. In both cases, iFentanylSnFR2.0 performs at least as well as expected in PBS buffers. FIG. 9C shows the iS-methadoneSnFR1.0 dose response to S-methadone in either 50% human sweat, 50% human saliva, or 25% mouse serum mixed with a remainder of 3×PBS pH 7.4. Final biosensor concentration is 100 nM in each case. It is noted that the final pH is unknown because each individual's sweat or blood sample is not itself buffered and the pH may vary across collections.

The response in serum has an unusually high S-Slope even prior to correction for ligand depletion. Further studies are required to determine if pH, high salt, or endogenous protein levels alter this dose response. Nevertheless, these data indicate the ability to perform therapeutic monitoring in multiple physiological fluids.

### Lyophilization and Stability

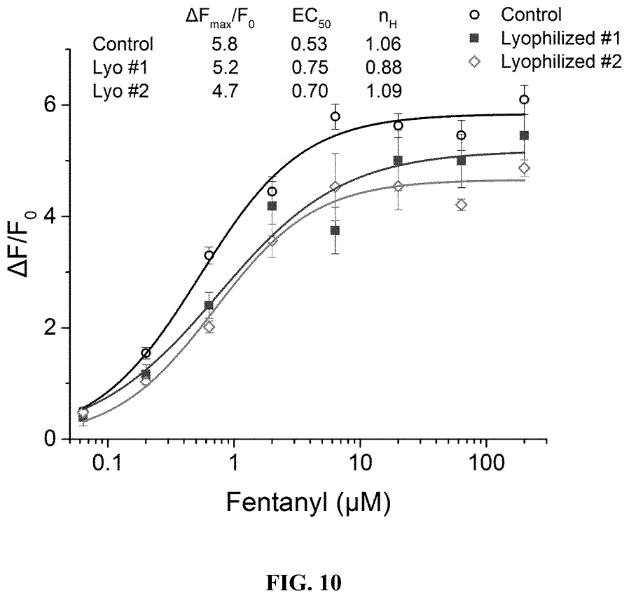

“iFentanylSnFR1.0 490G”, a predecessor biosensor one point mutation away from iFentanylSnFR2.0, was purified and lyophilized to yield a yellow-green powder. Storage at room temperature for 3 weeks and reconstitution in water led to a slight decrease in dynamic range and apparent EC50. FIG. 10 shows dose-response comparison between iFentanylSnFR1.0 490G stored at −80° C. and powder stored at room temperature for 3 weeks in the dark. These data suggest that iOpioidSnFRs can serve as quantitative environmental indicators (i.e. field testing) without refrigeration and can tolerate various methods of isolating and adhering protein to substrates (i.e. device fabrication).

### Live Cell Imaging Dose-Response Relations

iOpioidSnFRs, based on a bacterial periplasmic binding protein, have the advantage of minimal biochemical crosstalk within mammalian cells. These proteins are stable and soluble under physiological conditions, enabling targeting to subcellular compartments or the extracellular face of a cell. Exemplar biosensors are first tested as plasma membrane- (PM) and endoplasmic reticulum (ER)-targeted constructs transfected into HeLa cells. FIGS. 11A-11C show dose response of iFentanylSnFR1.0 localized to the PM (FIG. 11A) or the ER (FIG. 11B) of live HeLa cells during bath perfusion of fentanyl. With a Hill coefficient ˜1 and at [fentanyl] well below the EC50, a linear dose response is expected for iFentanylSnFR. FIG. 11C shows the dose-response from FIGS. 11A-11B plotted with linear fit (n=4 cells for each PM and ER dataset; SEM is given as error bars.). Epifluorescence imaging was performed with 470 nm LED illumination, 40× objective NA 1.0. Inset pictures show GFP fluorescence in representative HeLa cells. The data in FIG. 11C show good linear fit 50-200 nM fentanyl for both PM and ER signals.

iFentanylSnFR2.0 affords a detection limit in the single-digit nM range. FIG. 11D shows the most sensitive dose response to date that uses iFentanylSnFR2.0-PM in HeLa. A response to 5 nM fentanyl is observed that exceeds the HBSS buffer control. These data indicate that iFentanylSnFR2.0 can detect the lower end of the pharmacologically relevant range, the range that shows analgesic effects.

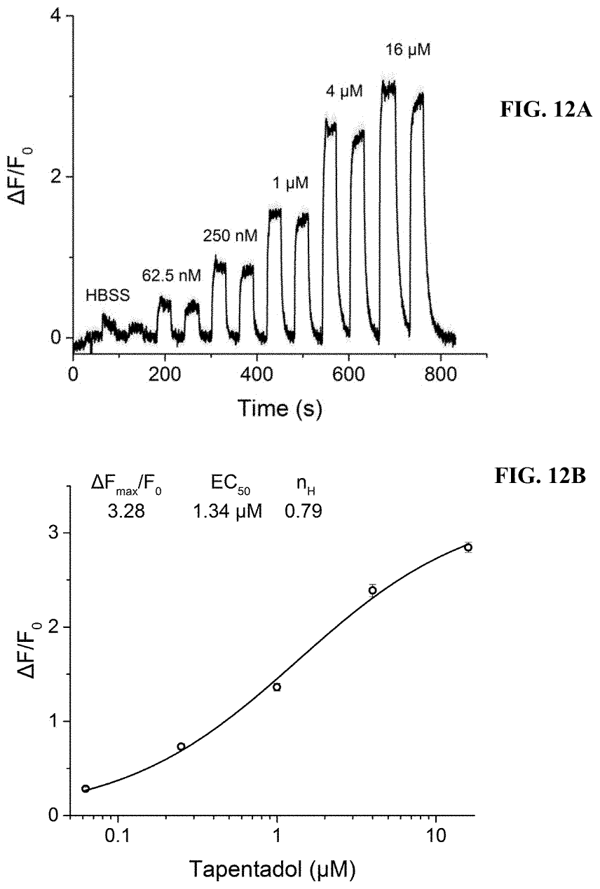

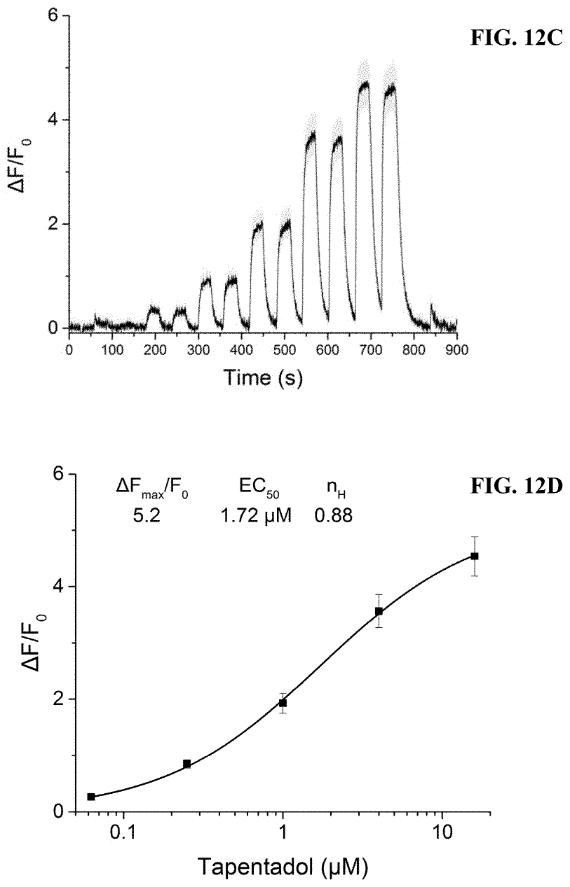

iTapentadolSnFR1.0 has also been transferred to PM- and ER-targeting constructs and successfully imaged in live HeLa cells. FIGS. 12A-12D show similar S-Slopes for the tapentadol dose-response at the PM and in the ER. FIG. 12A shows time-resolved signal of iTapentadolSnFR1.0-PM in HeLa during a stuttered-step program (n=7, SEM given as gray bounds). FIG. 12B shows averaged response of iTapentadolSnFR-PM in final ˜20 s window of 60 s pulses plotted against [tapentadol]. Hill fit and parameters given. FIG. 12C shows time-resolved signal of iTapentadolSnFR1.0-ER in HeLa during a stuttered-step program (n=7, SEM given as gray bounds). FIG. 12D shows averaged response of iTapentadolSnFR1.0-ER in final ˜20 s window plotted against [tapentadol]. Hill fit and parameters given.

### Stopped-Flow Kinetics

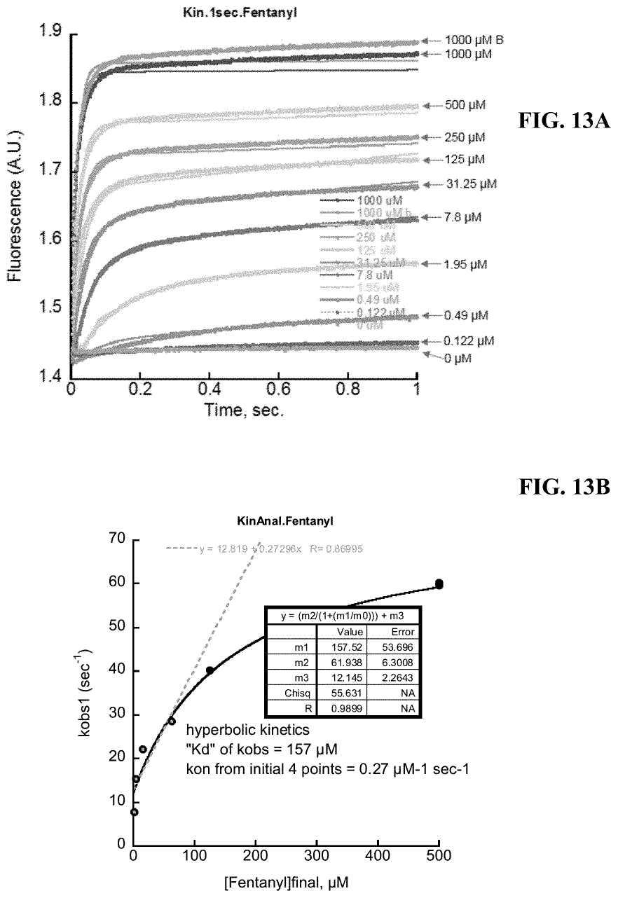

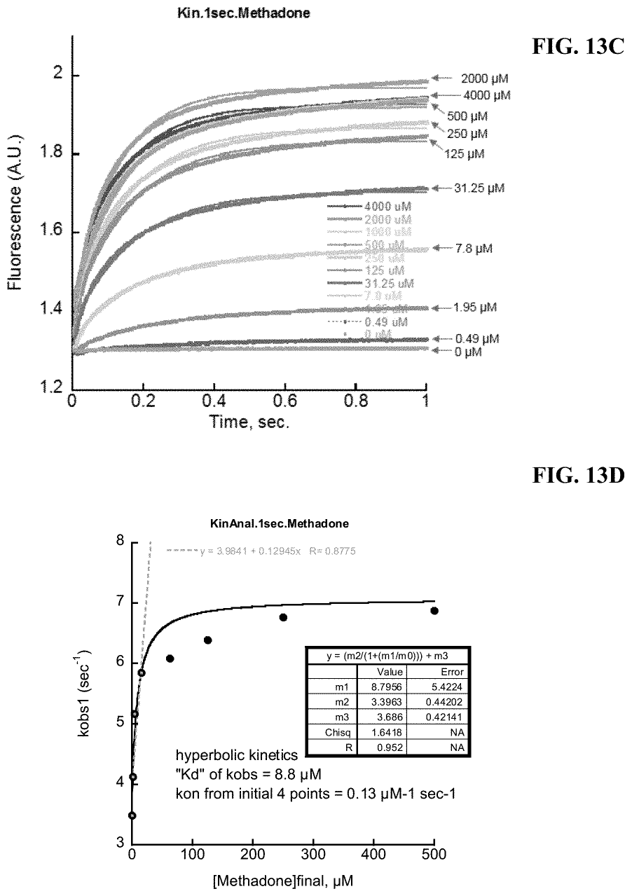

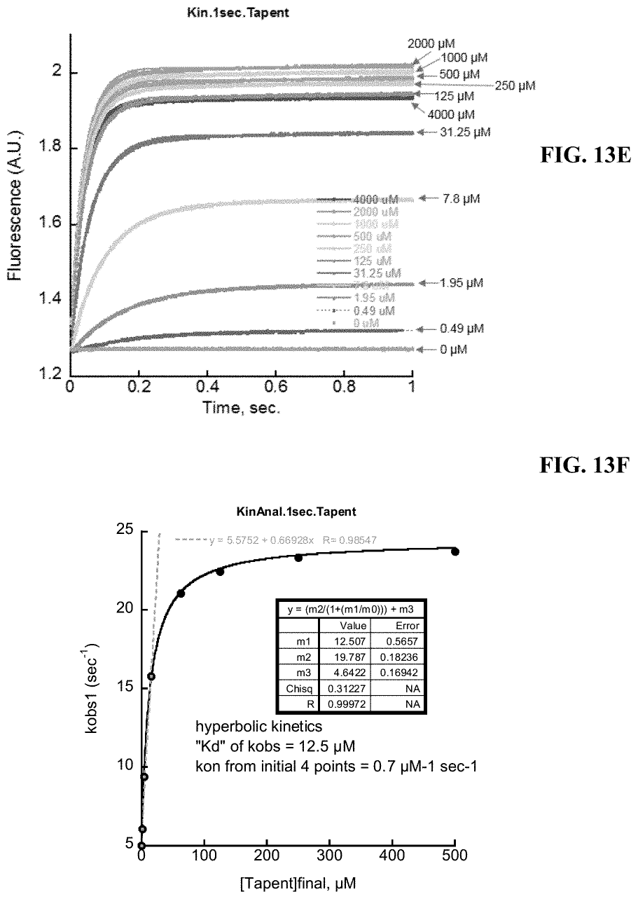

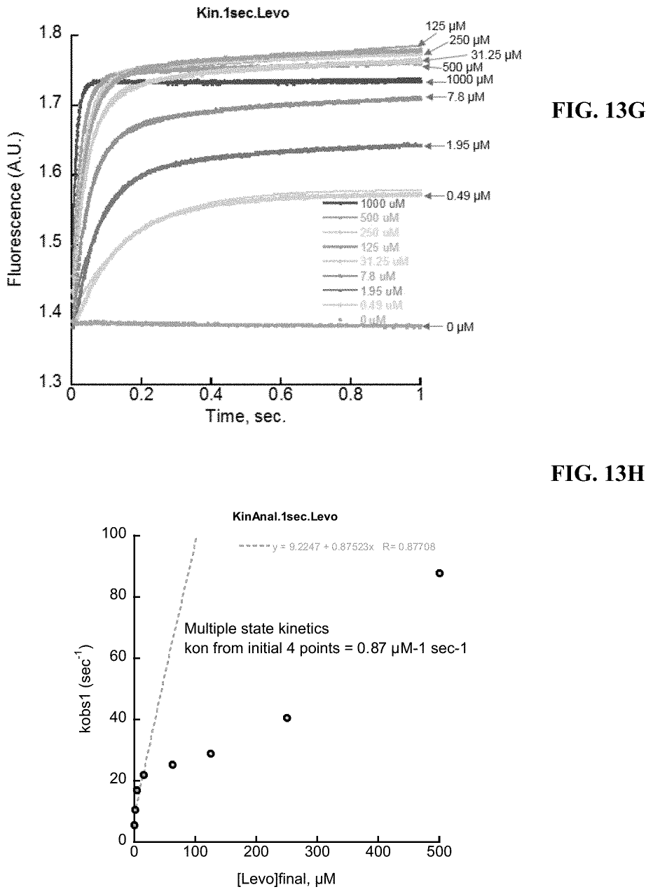

Stopped-flow kinetics were measured for the four exemplar biosensors where increasing [drug] was mixed with a constant purified [biosensor]. Data for the ˜1 s window (t=3 to 1000 ms) are shown in FIGS. 13A, 13C, 13E and 13G. In FIG. 13A, purified iFentanylSnFR2.0 mixed with increasing [fentanyl] as fluorescence was observed from 3 ms to 1000 ms. Raw fluorescence (arbitrary units) is plotted versus time. In FIG. 13B, Kon=0.27 μM-1 sec-1 was determined from data in FIG. 13A. In FIG. 13C, iS-methadoneSnFR1.0 purified protein mixed with increasing [racemic methadone] as fluorescence was observed from 3 ms to 1000 ms. Kon=0.13 μM-1 sec-1 was determined (FIG. 13D). In FIG. 13E, iTapentadolSnFR1.0 purified protein mixed with increasing [tapentadol] as fluorescence was observed from 3 ms to 1000 ms. Kon=0.7 μM-1 sec-1 was determined (FIG. 13F). In FIG. 13G, iLevorphanolSnFR1.0 purified protein mixed with increasing [levorphanol] as fluorescence was observed from 3 ms to 1000 ms. Kon=0.87 μM-1 sec-1 determined (FIG. 1311).

Within the 1 s window all biosensors show a biphasic response with responses increasing beyond 1 s. These data were sufficient to determine a Kon for each biosensor ranging from ˜0.1 to ˜0.9 μM⁻¹ sec⁻¹ (FIGS. 13B, D, F and H). The kinetics of these biosensors are sufficient for the ˜second to ˜minute resolution needed for pharmacokinetic studies in cells, animal models, and diagnostic devices for human monitoring.

### Acute Slice Imaging

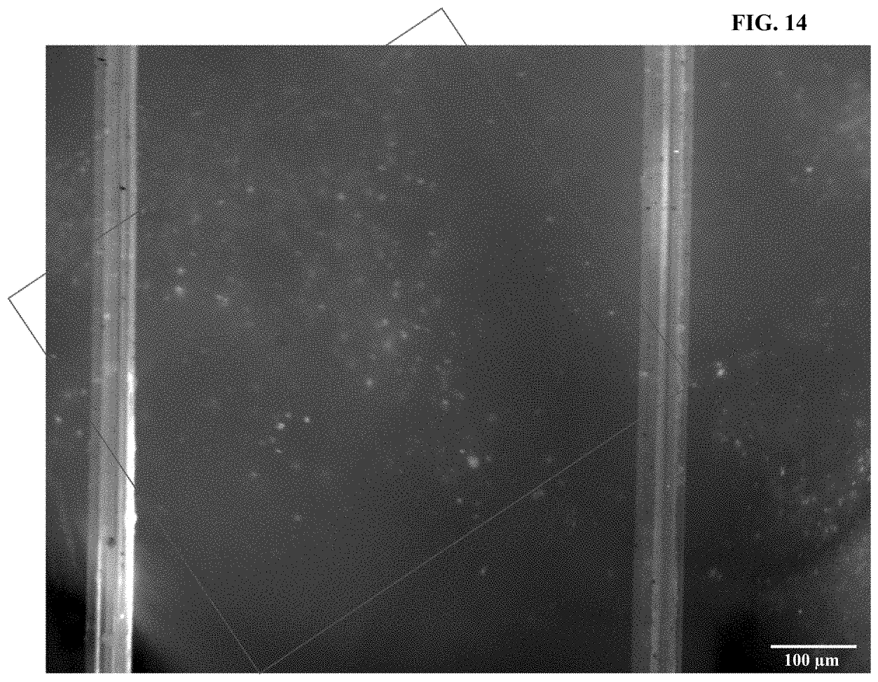

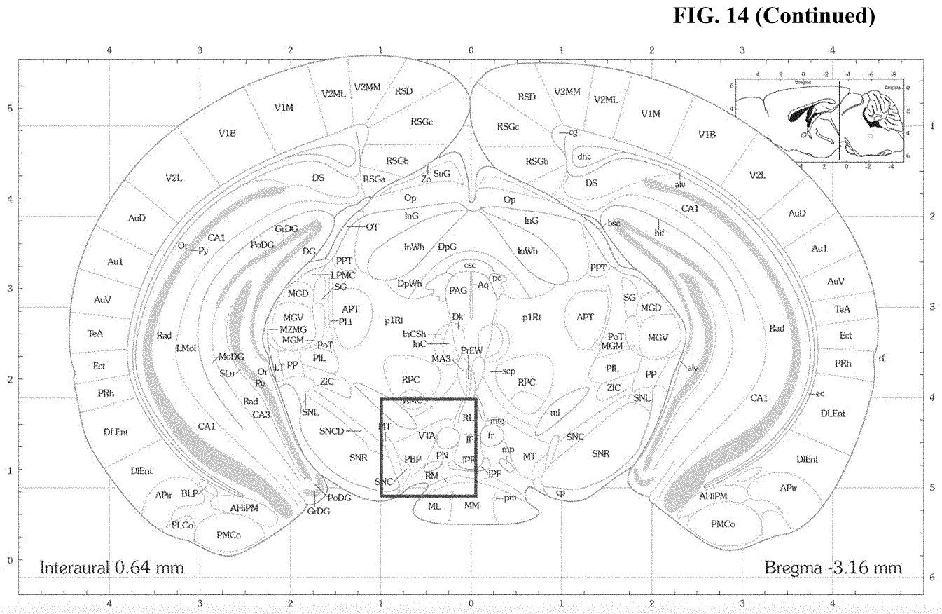

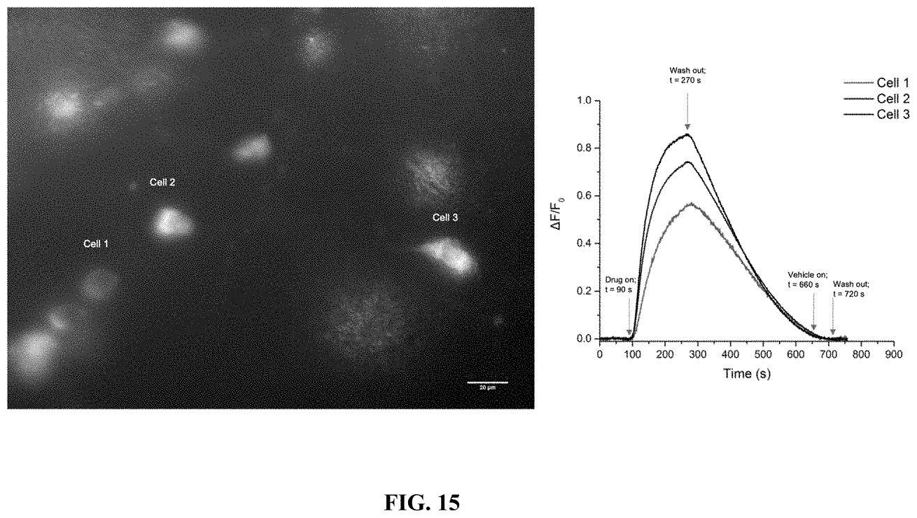

iFentanylSnFR2.0 was tested in the most relevant challenging biological environment: live brain tissue. AAV9-hSyn-iFentanylSnFR2.0-cytoplasm-WPRE was injected into the murine ventral tegmental area. After 2 weeks, the brain was sliced and imaged under drug perfusion. Expression was “strong” and widespread in the VTA (FIG. 14). The injection location for ventral tegmental area is shown in gray square (right panel of FIG. 14). The same ˜1 mm×1 mm field of view in slice image (left panel of FIG. 14) is also outlined in gray square. iFentanylSnFR2.0-cytoplasm expression is observed as “filling” cell bodies and the infected cells are widespread in the VTA. Biosensor in individual neuron cytoplasm could be visualized responding to bath perfused fentanyl (FIG. 15). Image of neuronal cell bodies expressing iFentanylSnFR2.0 (left panel of FIG. 15) show fluorescence throughout the cell with the exclusion of the nucleus. Bath perfusion of 1 micromolar fentanyl in ACSF led to a slow (˜minute) rise in fluorescence. The maximum response observed in one neuron was more than 80% increase in fluorescence with respect to initial baseline. Washout led to a similar slow (˜minute order) decay in signal. Perfusion of vehicle (ACSF in another line) led to no observed increase in signal from baseline. This is the first demonstration of a iDrugSnFR in acute brain slices. Critically, this experiment also represents the least amount of control over environment as live tissue demands particular molecular and gross environments suitable for transgene expression and imaging.

### Example 4

### Exemplary Additional Biosensors

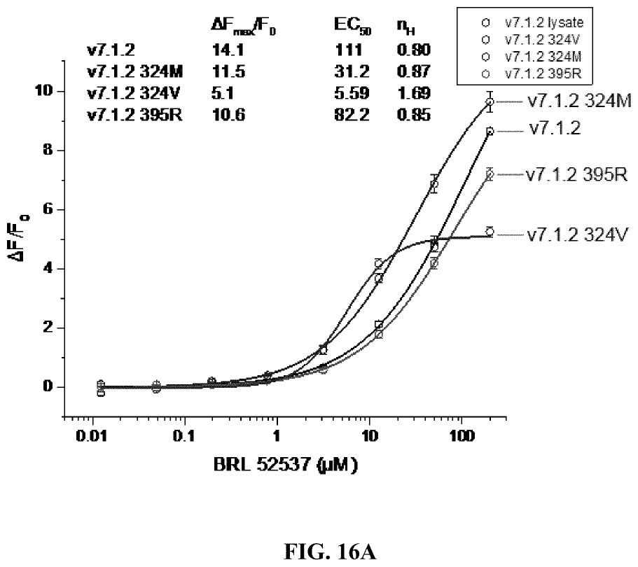

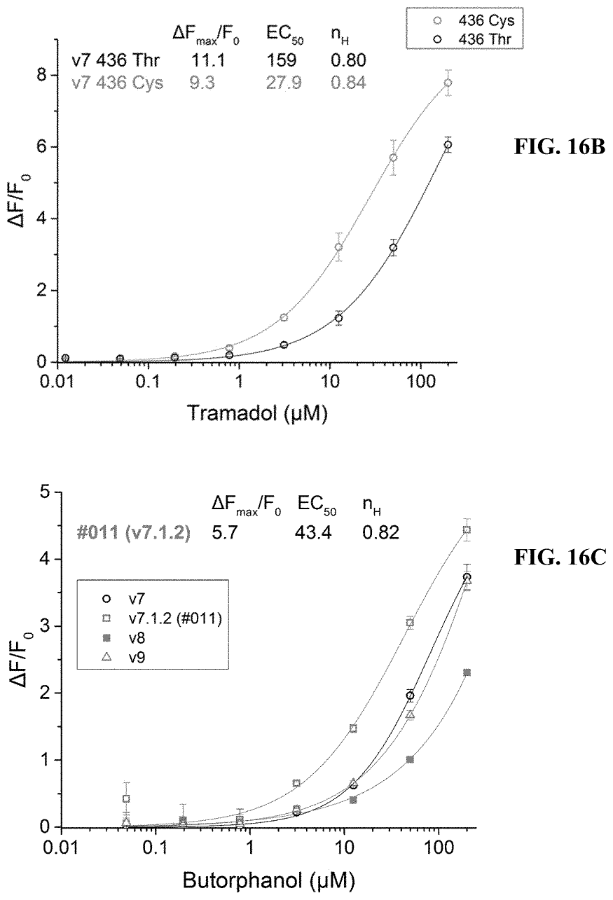

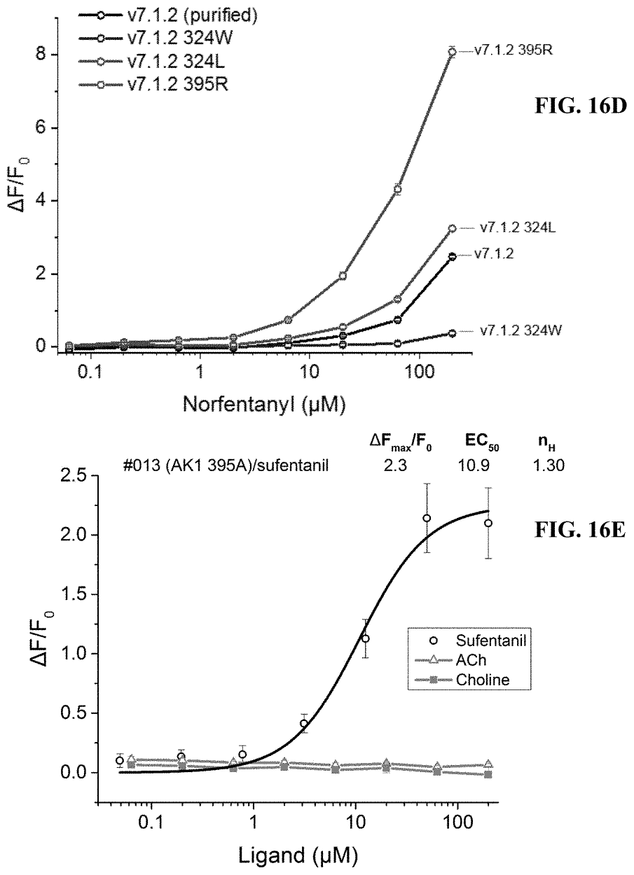

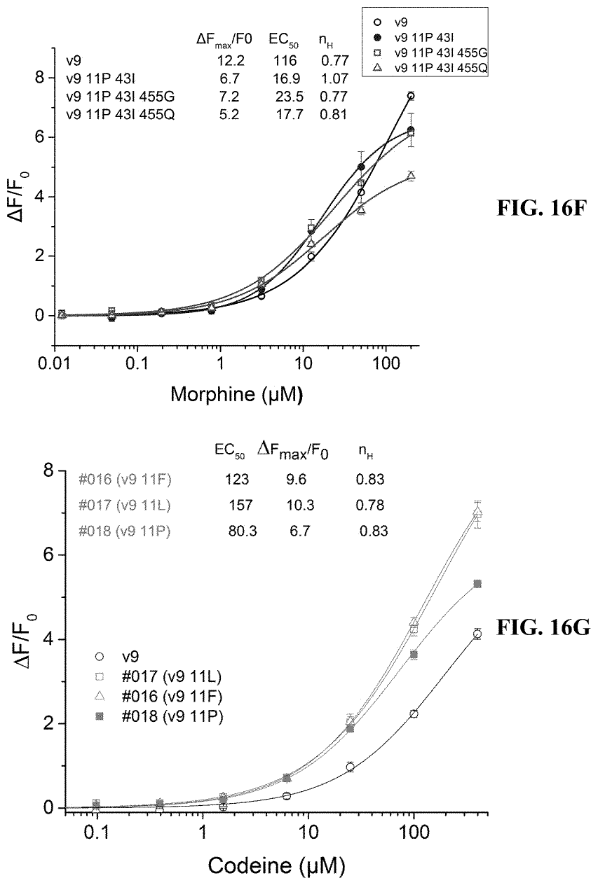

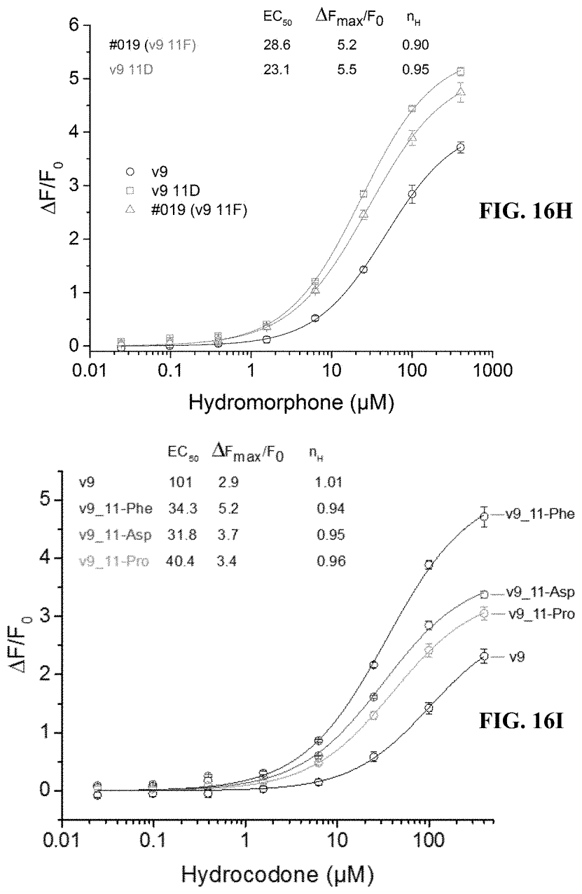

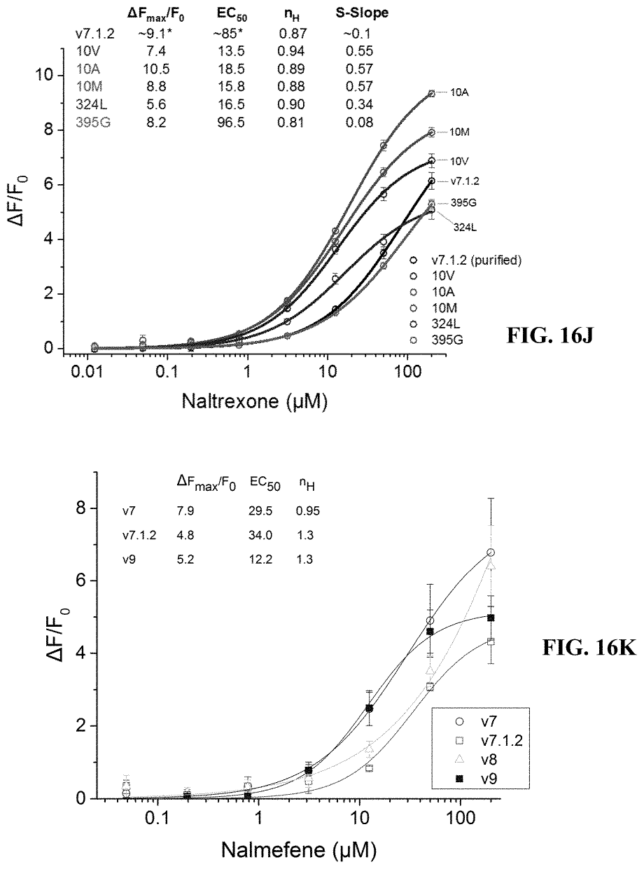

Several additional biosensor leads for opioids spanning μ and κ-opioids ligands are described in this example. These data demonstrate that OpuBC-based biosensors can be evolved for greater sensitivity in detecting a breadth of opioids. For example, BRL52537 is a highly potent, κ-opioid receptor-specific ligand detected by a biosensor lead (FIG. 16A) related to a biosensor lead for the μ-agonist morphine by six point mutations. In other words, the biosensor sequence/ligand/affinity “manifold” appears to be continuous across opioid pharmacological class. Yet, these biosensors hits and leads already provide complete selectivity in specific cases. For example, iFentanylSnFR2.0 does not detect norfentanyl and the leads for iNorfentanylSnFR exclude all hits for fentanyl (FIG. 2). Some hits of opioids are shown for comparison to leads for structurally related ligands.

FIGS. 16A-16K show dose responses for exemplary biosensor-opioid pairs. FIG. 16A shows dose responses for iBRL52537SnFR leads tested against BRL52537. FIG. 16B provides dose responses for iTramadolSnFR leads tested as diluted lysate against racemic tramadol. “v7 436 Thr” and “v7 436 Cys” correspond to SEQ ID NO: 9 and SEQ ID NO:10, respectively. FIG. 16C provides dose responses for the best two hits for butorphanol which do not overlap with the structurally similar levorphanol. “v7.1.2” corresponds to SEQ ID NO: 11. FIG. 16D provides dose responses for iNorfentanylSnFR leads tested as diluted lysate against norfentanyl. “v7.1.2 395R” corresponds to SEQ ID NO: 12. FIG. 16E provides dose responses for an iSufentanilSnFR lead which shows complete selectivity over acetylcholine and choline. “AK1 395M” corresponds to SEQ ID NO: 13. FIG. 16F provides dose responses for iMorphineSnFR leads tested as diluted lysate against morphine. This lead has been more extensively mutated compared to others shown here. “v9 11P 43I 455G” and “v9 11P 43I 455Q” correspond to SEQ ID NO: 14 and SEQ ID NO: 15, respectively. FIG. 16G provides dose responses for iCodeineSnFR leads tested as diluted lysate against codeine. These leads share the v9 11× sequence with the morphine family ligands. “v9 11 Phe”, “v9 11 Leu”, and “v9 11 Pro” correspond to SEQ ID NO: 16, SEQ ID NO: 17, and SEQ ID NO: 18, respectively. FIG. 16H provides dose responses for iHydromorphoneSnFR leads tested as diluted lysate against hydromorphone. “v9 11 Asp” corresponds to SEQ ID NO: 19. FIG. 16I provides dose responses for the same biosensor leads as in FIG. 16H, named now as “iHydrocodoneSnFR”, tested as diluted lysate against hydrocodone. “v9 11 Asp” corresponds to SEQ ID NO: 19. FIG. 16J provides dose responses for iNaltrexoneSnFR leads tested as diluted lysate against naltrexone. Some leads show appreciable sensitivity in the pharmacologically relevant [naltrexone] range. “v7.1.2 10V”, “v7.1.2 10A”, and “v7.1.2 10M” correspond to SEQ ID NO: 20, SEQ ID NO: 21, and SEQ ID NO: 22, respectively. FIG. 16K provides dose responses for iNalmefeneSnFR leads tested as diluted lysate against nalmefene. There is some overlap between hits for nalmefene and those for naltrexone.

The directed evolution results thus far point to key residues and strategies: (A) the effect of cholinergic-null cation-π mutations are preserved in other biosensors (e.g., iFentanylSnFR2.0 and iSufentanilSnFR). (B) the last residue of linker 2 (324P) is rarely ideal for a given opioid-biosensor pair and may be mutated to increase dynamic range. (C) Mutations at positions 10, 11, and 395 are often advantageous to detect opioids. (D) Iterative single residue site-saturation mutagenesis appears to yield progressively improved biosensors in all cases tested thus far. In other words, the biosensor fitness landscape does not appear to have deep local minima.

### Example 5

### Design of Exemplary S-Methadone Biosensors

In this example, a fluorescent biosensor of S-methadone, “intensity-based S-methadone sensing fluorescent reporter” or “iS-methadoneSnFR1.0”, is reported. The biosensor can be genetically encoded in mammalian cells or used as isolated protein for diagnostic measurements. The biosensor scaffold comprises a mutated periplasmic binding protein, OpuBC, fused to a circularly permuted GFP (see e.g., FIG. 1A). The resulting biosensor detects S-methadone across the pharmacologically relevant range with an EC50 of 3.17 μm and dynamic range of 1530% change from fluorescence baseline.

In some embodiments, the biosensor scaffold has an OpuBC from Thermoanaerobacter sp. X513, which is a homologue of OpuBC from B. subtilis (Du '11), fused to a circularly permuted GFP (FIG. 1A). OpuBC binds choline in nature. OpuBC variants in biosensors of nicotine and acetylcholine have been previously reported (Shivange '19; Borden '20). One variant, iNicSnFR3a (SEQ ID NO: 27), served as the parent for this work's directed evolution. Through directed evolution, OpuBC's selectivity has been switched in favor of S-methadone and against endogenous neurotransmitter including cholinergic ligands, and other neural drug classes including other members of the opioid class.

### Directed Evolution Strategy

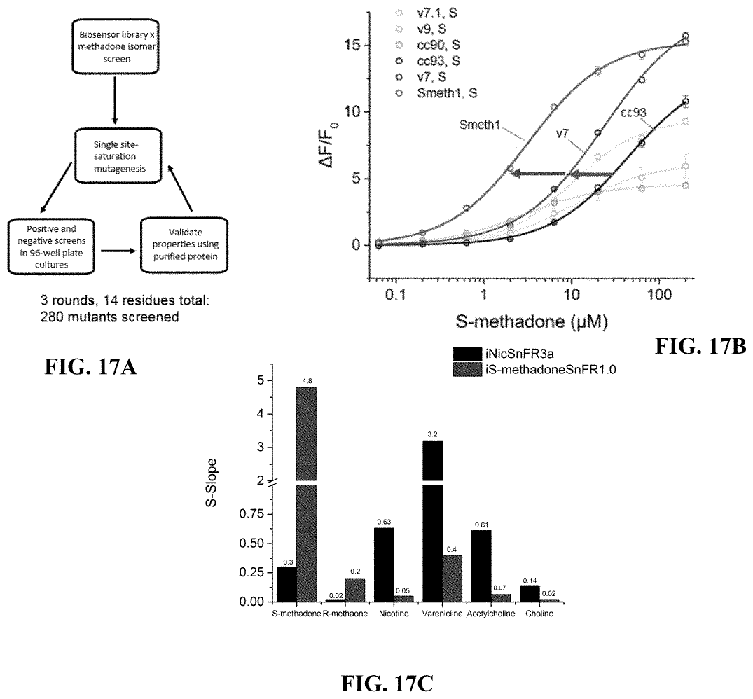

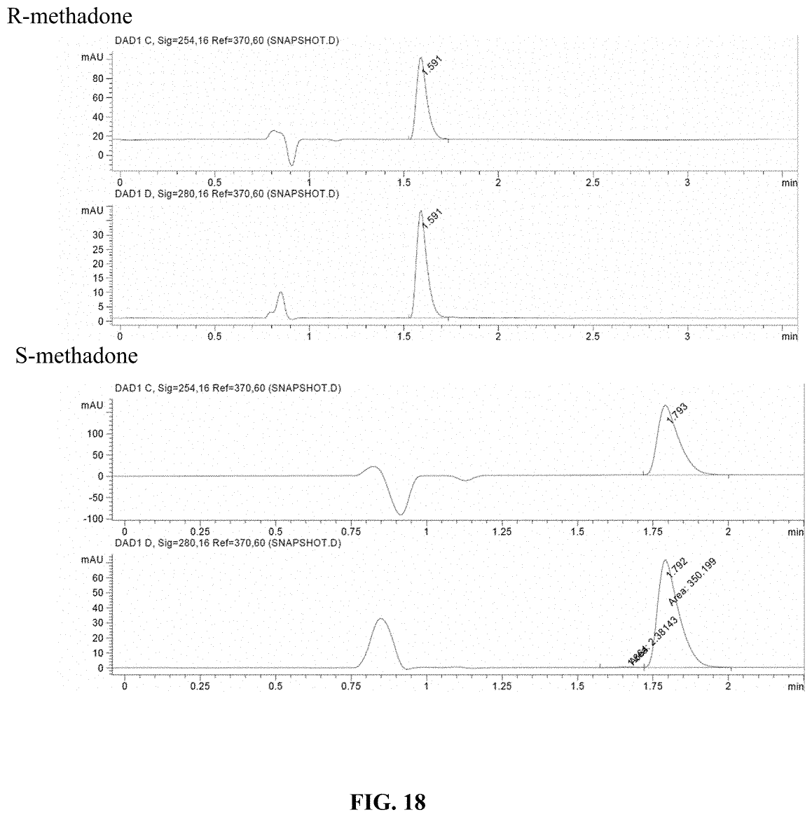

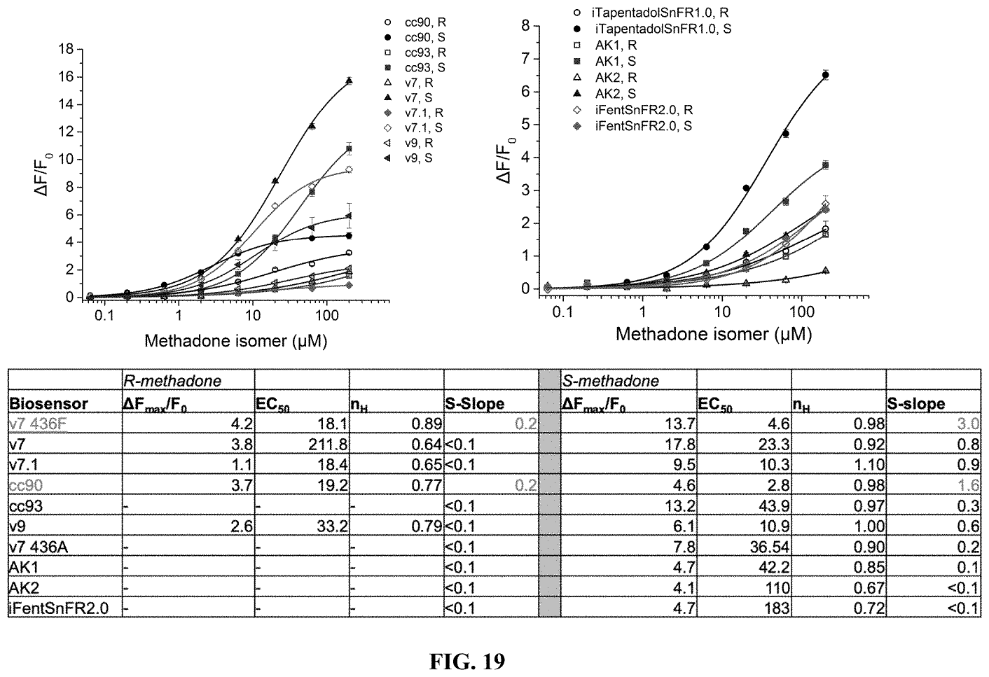

Directed evolution strategy employed in this example consists of (1) lead discovery by screening each methadone enantiomer against a family of iNicSnFR3a variants and (2) iterative site-saturation mutagenesis to select for S-methadone and select against cholinergic ligands (FIG. 17A). After a promising variant was identified, iterative single-position site-saturation mutagenesis were carried out. Variants with either best increase in methadone and/or decrease in cholinergic sensitivity were taken forward at each round. Chiral resolution was first performed on racemic methadone to isolate (+)-S-methadone and (−)-R-methadone with analytical purity and 99% enantiomeric excess (FIG. 18). Purified cholinergic biosensors were screened against each methadone enantiomer for their fluorescence response (FIG. 19). A dose response across ˜3 orders of magnitude of [S-methadone] can be fit with a Hill curve to determine an EC50 and ΔFmax/F0. A metric for biosensor sensitivity was previously defined: S-slope=d(F/F0)/(d[ligand]) (Bera '19). For Hill coefficient ˜1.0, the S-slope=ΔFmax/F0/EC50. This metric combines the two key factors for biosensor performance, dynamic range and affinity, into a single metric comparable across variants. A variant of iNicSnFR3a, iNicSnFR3b, provided the best responses to both R-methadone and Smethadone; however, the maximum S-Slope found was only 0.3 for the S-methadone and iNicSnFR3b pair. Although OpuBC had no pressure for enantioselective binding of its ligand in nature (choline is not chiral) let alone a synthetic opioid, all biosensors screened displayed enantioselectivity for S-methadone (FIG. 18).

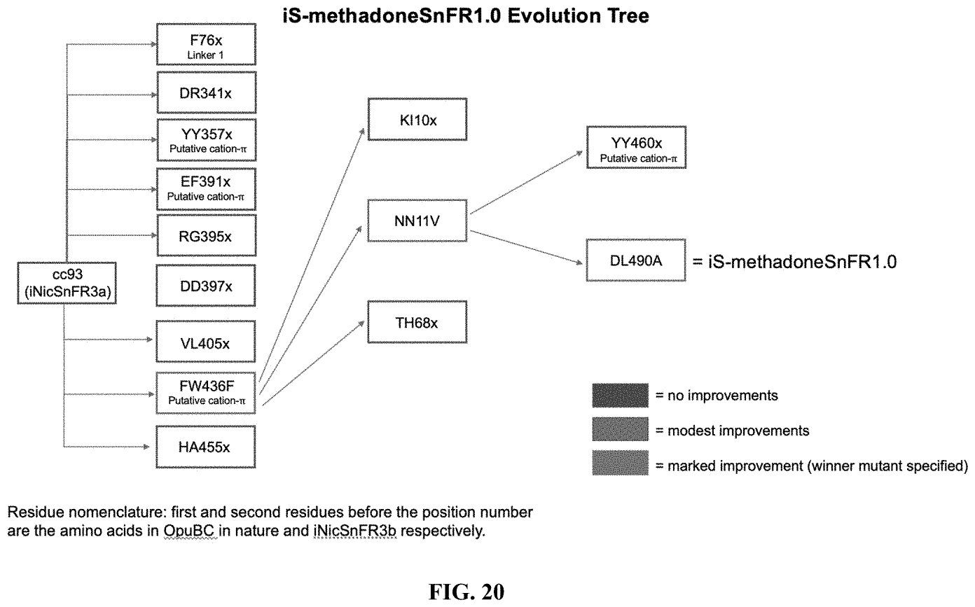

S-methadone biosensor was elected for further development to take advantage of this binding pocket bias. iNicSnFR3b was subjected to several rounds of directed evolution to yield a selective and sensitive biosensor of S-methadone. Screens were conducted in response to R-methadone, S-methadone, choline, and acetylcholine. Winning variants were selected based on both an increase in sensitivity to methadone and a decrease in sensitivity to cholinergic ligands. Mutation sites was selected based on a crystal structure of iNicSnFR1 (PDB: 6EFR; SEQ ID NO:26) and directed evolution results in generating iNicSnFR3a (Shivange '19). In the initial round, most sites yielded null results except for a conservative W436F mutation at a putative cation-π residue (FIG. 20). In the subsequent round, “second-shell” residue 11 was mutated from Glu to Val, creating more space next to another putative cation-π residue. Notably, iNicSnFR3a and 3b differ by a single point mutation at this residue. Finally, the third round discovered a Leu to Gly mutation at the distal residue 490, allowing for greater flexibility in the hinging of OpuBC. All three mutations share a reduction in side chain volume, likely accommodating the larger steric bulk of methadone.

The resulting iS-methadoneSnFR1.0 had a ˜16-fold improvement in sensitivity over iNicSnFR3a (FIG. 17B). FIG. 17B shows fluorescence responses of various biosensor variants to S-methadone. iNicSnFR3a (black) had several variants (faded curves), of which one had markedly better sensitivity, owing to a N11E mutation (blue). This lead was evolved to the final iS-methadoneSnFR (red) which included re-optimization at position 11. Only the final biosensor has sufficient sensitivity at 1 μM (vertical blue line), the relevant maintenance concentration. Notably, the final OpuBC variant displays a sensitivity for S-methadone that eclipses the sensitivity for any of the original cholinergic ligands. Furthermore, directed evolution yielded stark shift in selectivity (FIG. 17C). Critically, iS-methadoneSnFR1.0 has sufficient selectivity against cholinergic ligands with near-zero response for ACh and choline at their physiological max concentrations, 10 and 20 uM respectively for choline and even lower for ACh. Furthermore, iS-methadoneSnFR1.0 does not detect varenicline or nicotine in their pharmacologically relevant concentration 0 to 100 nM (Faessel 2010) and ˜25 to ˜500 nM respectively.

Binding Between iS-methadoneSnFR1.0 and S-Methadone

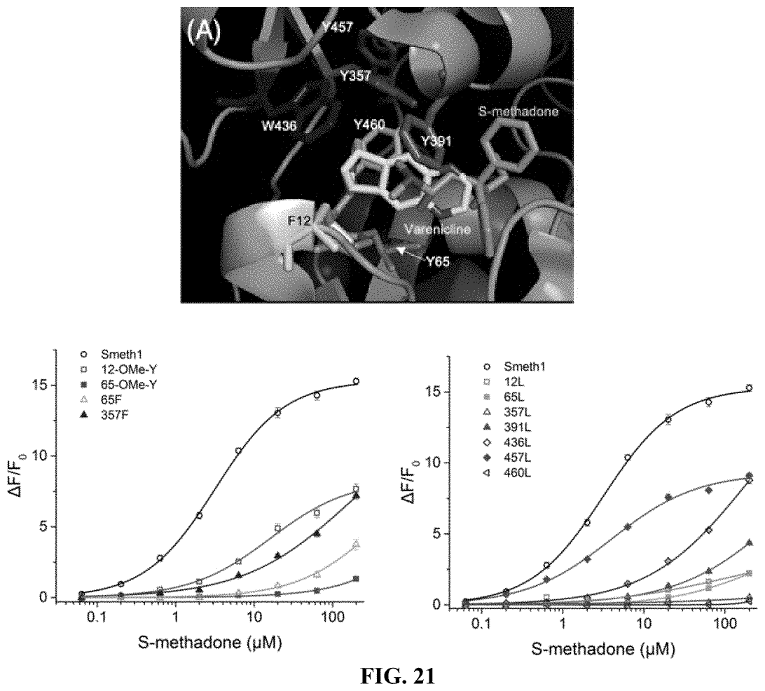

A crystal structure of iNicSnFR3a with varenicline bound (PDB: 7S7T, Nichols 2021) was recently reported. Therein, the aromatic rings of Y65 and Y357 are parallel, and varenicline's tertiary nitrogen lies on the midpoint of an axis between the centroids of the two aromatic side chains, making cation-π interactions with both (FIG. 21, panel A). FIG. 21, panel A shows a cation-π interaction with Y65 and 357Y axis. S-methadone was docked into 7S7T; its amine is pointed toward the binding pocket but does not penetrate to engage Y65/Y357. The mutagenesis study for S-methadone at iS-methadoneSnFR also shows the primacy of Y65 and Y357 (FIG. 21, panel B). FIG. 21, panel B shows fluorescence dose response of cation-π residue Leu mutants. Furthermore, the Y12-OMe-Y mutation produces only a modest increase in EC50, in contrast with the dramatic loss of function for the Y12L mutation (FIG. 21, panel C). FIG. 21, panel C shows Unnatural amino acid (O-methyl tyrosine, “OMe-Y”) incorporation probed steric and H-bonds at residues 12 and 65. Aromatic residue screen shows Y65 is a requirement whereas Y357F is better tolerated. This is consistent with the idea that aromaticity at the position 12 side chain is also important for the binding of S-methadone. The docked S-methadone pose shows one of the N-methyl groups of S-methadone just 4 angstroms from the centroid of F12, on an axis perpendicular to the plane of F12 (FIG. 21, panel A). In this pose, the N-methyl groups of S-methadone lie ˜ 4.6 and 5.5 angstroms from the aromatic groups of Y357 and Y65, respectively, slightly further than the distance from the beta carbons of varenicline to these two groups. The accepted W436F mutation in the biosensor and relatively tolerated F436L mutation indicate reducing volume residue 436 allows for closer access to Y65/Y357. Methadone has only one polar group, a carbonyl, in addition to its tertiary amine. Given the crystal structure precedent, the mutational study herein, and the relative favorability of cation-π interaction to π-π stacking, a cation-π interaction is likely present in iS-methadoneSnFR. These results suggest that OpuBC has an aromatic amine-binding pocket that may be tuned to the remainder of the ligand's steric bulk and functional groups to switch selectivity.

Selectivity and Sensitivity Characterization of iS-methadoneSnFR1.0

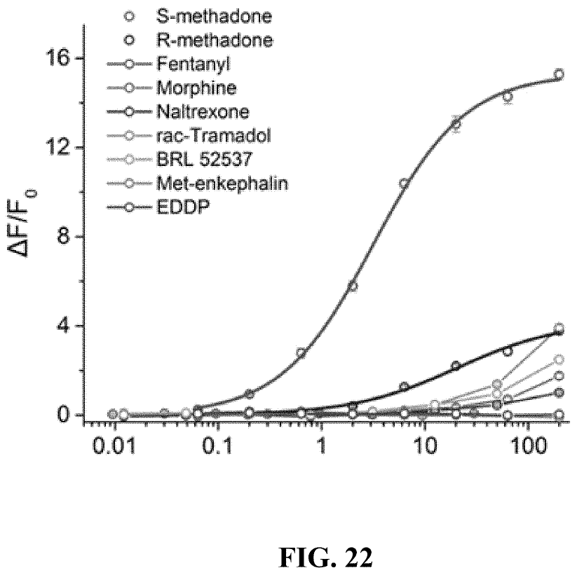

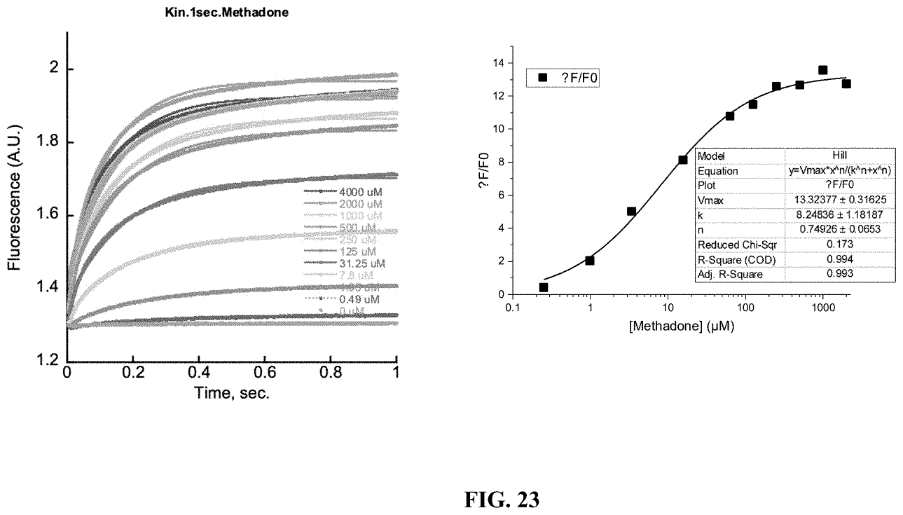

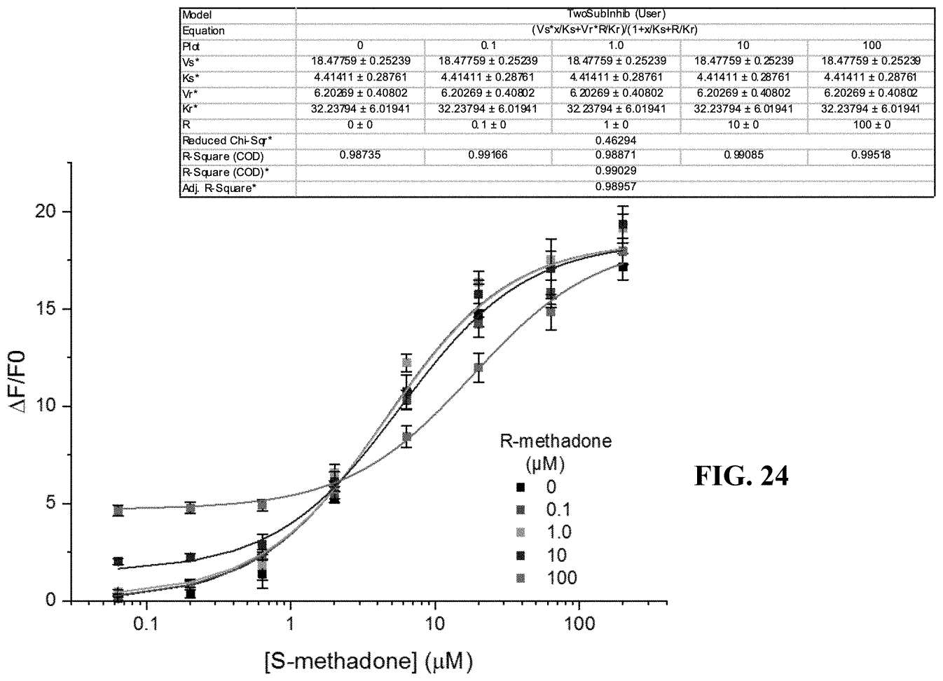

Various tests have shown that iS-methadoneSnFR1.0 satisfied selectivity in dose-responses and biophysical criteria for a well-behaved biosensor. Fluorescence dose response shows an excellent dynamic range, ΔFmax/F0˜15, and EC50 near the relevant plasma concentrations for maintenance therapy. Isothermal titration calorimetry determined a Kd of 1.9 μM, in good agreement with the fluorescence EC50 (FIG. 5B). ITC also demonstrated a single binding site (stoichiometry=0.92) with an entropically-driven conformational change. iS-methadoneSnFR has little or no response (S-Slope <0.1) to other neurotransmitters (FIG. 7B) and other opioids with minimal response to R-methadone (FIG. 22). FIG. 7B shows iS-methadoneSnFR vs endogenous neurotransmitters. No appreciable response is observed against non-cholinergic ligands. Minimal response is observed for ACh and choline in their physiologically relevant concentrations at ˜1 μM and ˜10 μM respectively. FIG. 22 shows fluorescence dose response of) iS-methadoneSnFR vs other clinically used opioids. The response to R-methadone is near zero at ˜1 μM but is appreciable at ˜3 uM. Otherwise, weak or no responses are observed well beyond clinical and abuse concentrations for other drugs. The S-slope for S-methadone is ˜20× that for R-methadone. When titrating R-methadone into the S-methadone dose response, fluorescence is elevated with increasing R-methadone but all responses converge at the ΔFmax/F0 for S-methadone alone (FIG. 24). Moreover, stopped flow kinetics were obtained using racemic methadone (FIG. 23). An apparent kon of 0.13 μM-1s-1 was calculated from the 1-second stopped flow data (FIG. 13D). The last 10 ms of the 1 second stopped flow data was fit by a Hill equation to find an EC50˜ 8 μM (FIG. 23) for the racemate, roughly double the EC50 in purified protein with S-methadone alone. These steady-state and kinetic data indicate that the response to a racemic mixture is dominated by S-methadone.

The traditional blood draw method to study PK in human subjects is invasive and inaccessible to many. Recent developments in sweat and saliva monitoring enable non-invasive methods for at-home testing (Chung '19; Soares Nunes '15). iS-methadoneSnFR1.0's aqueous solubility and selectivity enables its use as a diagnostic tool. The biosensor was tested in 1:1 PBS:biofluid samples and robust responses was found in the pharmacologically relevant concentration ranges (FIG. 9C). FIG. 9C shows iS-methadoneSnFR response in biofluids: 1:1 mixture of drug/biosensor in 3×PBS pH 7.4 with human sweat, human saliva, and 1:3 mixture with mouse serum (no pH adjustment of any biofluid). Final [biosensor] was 100 nM. SEM given as error bars.

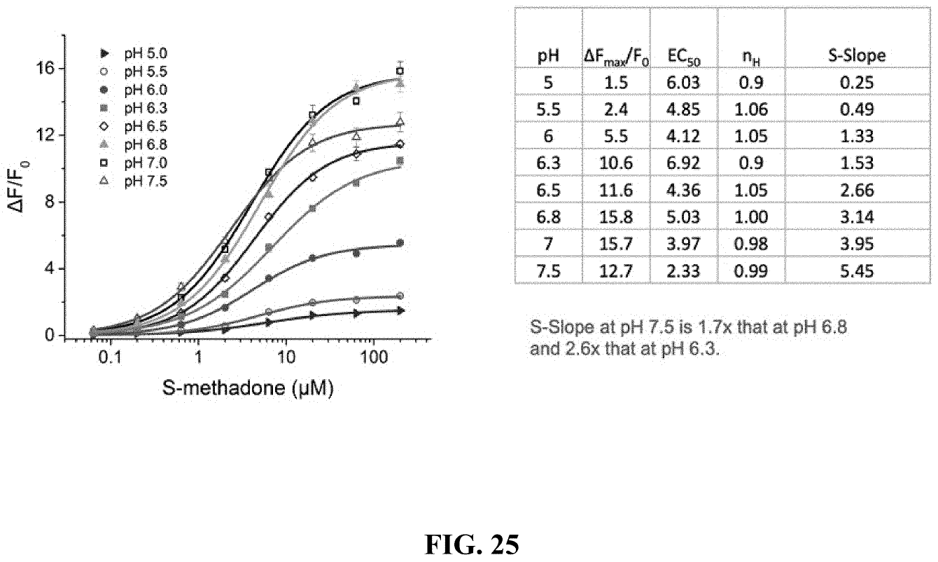

iS-methadoneSnFR1.0, like all GFP-based biosensors, has a pH-sensitive dose response: the S-Slope drops to ˜1.3 at pH 6.0 and 0.25 at pH 5.0 (FIG. 25). Because human biofluids, particularly sweat, may have variable and/or acidic pH, a higher strength buffer was used. The dynamic range was found to be limited in the higher salt conditions, so a tradeoff between pH control and salinity was necessary. Still, the responses at ˜1 μM and below in the biofluids provide ˜200% dynamic range.

Neural Studies of iSmethadoneSnFR1.0

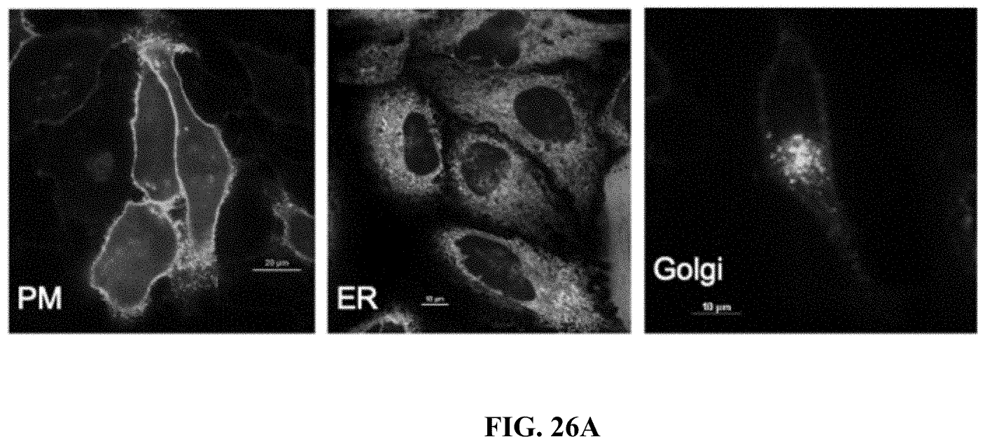

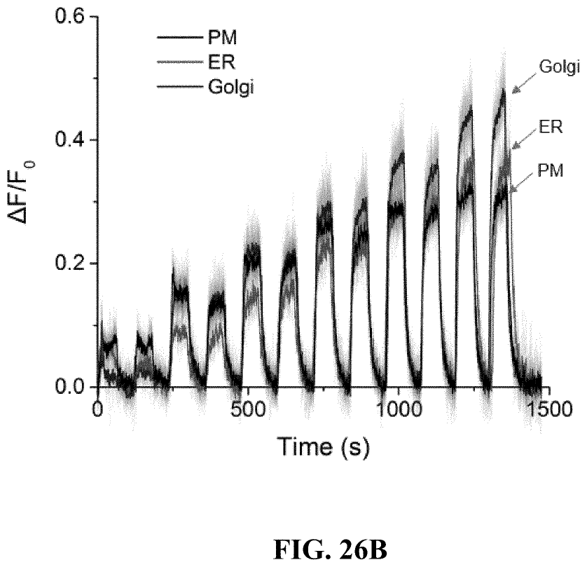

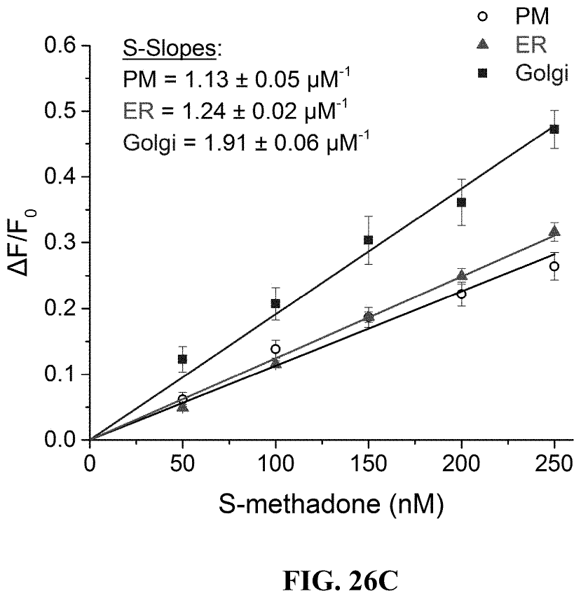

At the subcellular level, opioid drugs, but not endogenous opioids can act as pharmacological chaperones in the endoplasmic reticulum, perturbing the folding and trafficking of their receptors (Petäjä-Repo '14). Opioid drugs can activate their receptors in endosomes and the Golgi apparatus (Stoeber '18). iSmethadoneSnFR1.0 was targeted to the plasma membrane, endoplasmic reticulum, and Golgi apparatus using address sequences. Confocal imaging under 100× magnification shows proper targeting based on organelle morphology (FIG. 26A). Widefield imaging during bath perfusion of drug provided time-resolved [drug] waveforms (FIG. 26B). Pulses of S-methadone (0 to 250 nM in 50 nM steps) was to sample a linear start to the dose-response relation. The Golgi shows the largest S-slope across all compartments, 1.7× that of PM, despite the lowest pH of the three. When correcting for pH dependence of S-Slope, the accumulation factor is 2.9× to 4.4× across the Golgi pH range of 6.3 to 6.8 (FIG. 24). These results indicate (1) ample S-methadone is available in the ER for potential chaperoning, (2) activation of opioid receptors in Golgi should be considered alongside activation at the PM with respect to cellular tolerance, and (3) accumulation of a weak base in acidic compartments may lead to intensified G-protein coupled signaling.

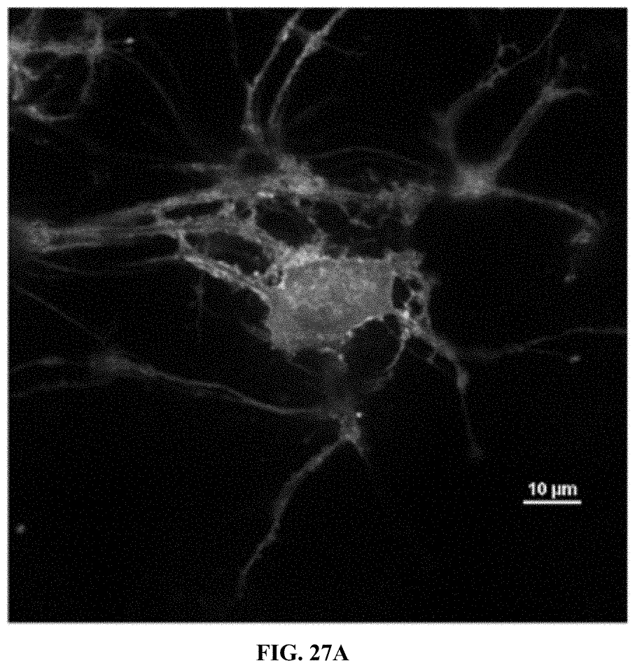

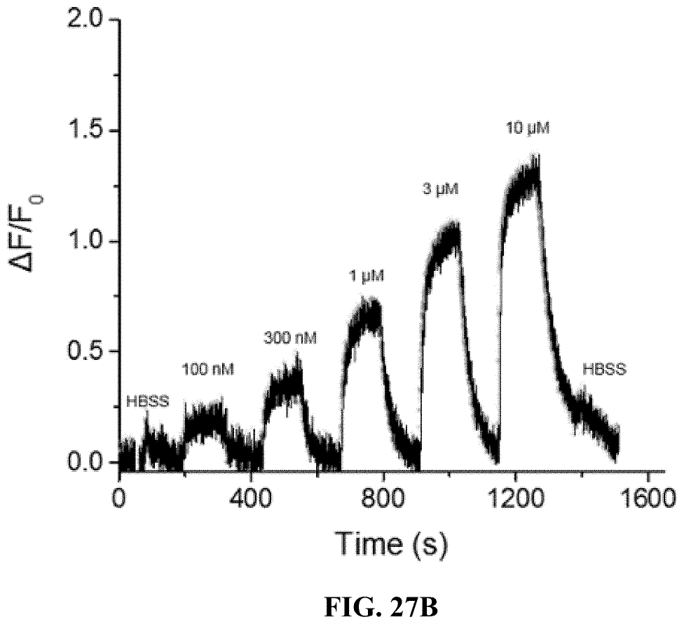

iS-methadoneSnFR1.0-PM was validated for use in mouse neurons for eventual use in rodent models of addiction. The iS-methadoneSnFR1.0 gene was cloned into a pAAV backbone with human synapsin promoter to limit expression to neurons. The pAAV was packaged into an adeno associated virus (AAV) with a PHP.eb capsid and purified by density gradient ultracentrifugation (Challis 2019). Primary hippocampal neurons were transduced with the AAV for two weeks and then imaged. FIG. 27A shows the spinning disc confocal imaging of mouse hippocampal neuronal culture transduced with PHP.eb-hSyn-iS-methadoneSnFR-PM-WPRE. FIG. 27B shows time-resolved waveforms during bath perfusion of S-methadone (n=12, SEM as gray bounds). Confocal imaging confirmed localization to the plasma membrane (FIG. 27A). Widefield imaging under drug perfusion demonstrated detection across the pharmacologically relevant range (FIG. 27B). These results demonstrate iS-methadoneSnFR may be deployed in mouse tissue with cell-type specificity and good localization at the plasma membrane in both cell body and neurites.

### Example 6

### Exemplary Mutated Chromophore with Minimized pH Sensitivity

In this example, biosensors comprising a mutated chromophore demonstrate minimized pH sensitivity.

The primary source of artifacts in GFP-based biosensors is transient change in local pH. GFP's tyrosine-based chromophore has a phenolate with pKa˜6 whose protonation state modulates fluorescence. In the experiments with iOpioidSnFRs described herein, the buffer volume constitutes at least 50% of the solution volume. This method is suitable for experimental and diagnostic applications. To increase robustness, artifacts in the biosensor signal was controlled or minimized. mTurquoise is a fluorescent protein with a tryptophan-based chromophore with pKa˜3, providing a largely pH-insensitive response across the physiologically encountered pH range. It has been verified that a glutamate-binding biosensor with a mTurquoise reporter (iGluSnFR-mTurquoise, Marvin '18) enables detection across pH 4.5-8.0.

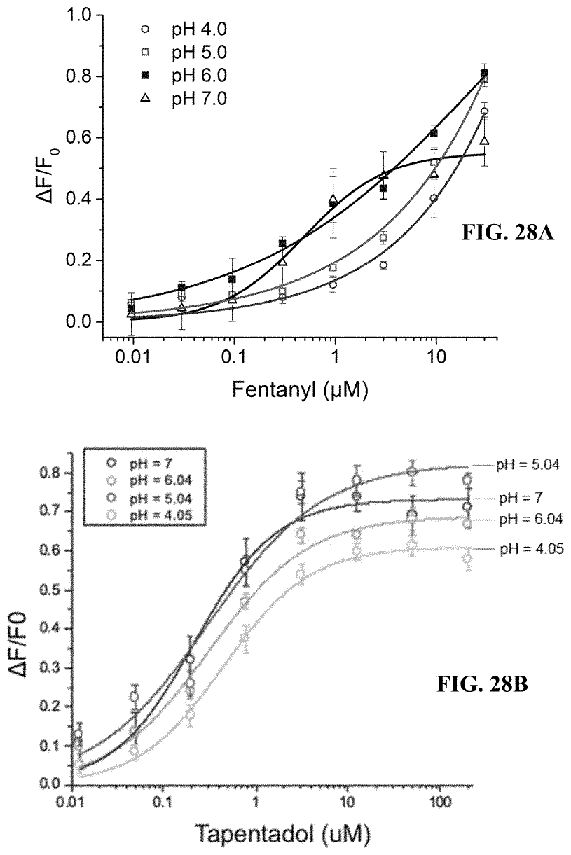

FIGS. 28A-28B show dose response with mTurquoise variants of iFentanylSnFR2.0 and iTapentadolSnFR1.0 respectively.

When the chromophore is mutated, the linkers connecting the PBP must be mutated accordingly to increase dynamic range; although most of the PBP's binding pocket is preserved in this process, it is likely that linkers must also be optimized to preserve sensitivity (Marvin '18). Site-saturation mutagenesis of the linkers can generate iOpioidSnFR-mTurquioise constructs with sufficient S-Slope for various applications. These data demonstrate (A) significant improvements in the mTurquoise variants after SSM in the second linker (residues 321-324) and (B) resulting variants provide less pH-sensitive responses. These biosensors would require less stringent pH control of the sample buffer for robust measurements. Therefore, the application of iOpioidSnFRs in cellular studies could be extended to acidic organelles.

### Example 7

### Exemplary Materials and Methods

The following exemplary materials and methods are used in the preparation and practice of the embodiments disclosed herein.

Animal use statement: Cull C57BL/6 mice were used for terminal cardiac puncture procedure to collect blood. Animal care was conducted in accordance with the guidelines for care and use of animals provided by the National Institutes of Health, as well as our IACUC protocol #1386 at the California Institute of Technology. Animals had been kept on a standard 12 hr light/dark cycle and given food and water ad libitum.

Chiral Resolution of Methadone: Racemic methadone was purchased from Sigma Aldrich. A saturated solution was prepared in ethanol with ˜0.1% triethylamine. Supercritical fluid chromatography was run using a CHIRACEL® OJ-H stationary phase column (Chiral Technologies). The resulting fractions of each isomer were combined and then each recrystallized from EtOH. The isomers were identified by their optical rotation in water (Howe '48).

Opioid panel preparation: Compounds were purchased from Sigma Aldrich, Cayman Chemical, Tocris, and Santa Cruz Biotechnology. A 2 mM solution of each compound was prepared by dissolving in 3×PBS pH 7.0 with 0, 25, 50, 75% DMSO or in neat DMSO. Nearly all μ-opioid drugs had sufficient water solubility to not require any DMSO. 1 mL of each solution was added to a single well in a 96-deep well plate. Each of the buffers used to prepare the solutions was added to three wells (i.e. negative control: no drug). When not in use, the deep well plate was sealed and stored at −20° C.

Virtual Mutagenesis and Docking: AutoDock Vina was used to perform docking. iNicSnFR3a crystal structure was obtained from Protein Data Bank (ID: 7S7T). Chimera was used to make the 11V, 436F, and 490G mutations by selecting the most favorable rotamer. The structure was then prepared in AutoDock tools by removing waters, adding polar hydrogens, and assigning Gasteiger charges. Ligands were allowed torsional freedom in the docking routine. The highest scoring conformation with methadone's amine directed into the binding pocket was chosen for analysis. To validate this method, varenicline was docked back into iNicSnFR3a and found to overlap with ˜1-2 angstrom deviation between each of the three nitrogens in the highest scoring structure.

Biosensor expression by autoinduction: pHHMI plasmids bearing a biosensor gene were transformed into chemically competent BL21 DE3 cells and grown on ampicillin selection plates overnight at 37 C. Autoinduction LB is prepared according to Studier 2005 with ampicillin (100 mg/L). A single colony is picked to inoculate each vessel with autoinduction media. The vessel is incubated at 30 C with shaking at 250 rpm for 28-30 h, shielded from light. Biosensor expression was evident from a yellow-green colored culture.

Unnatural Amino Acid Mutagenesis: “Amber codon suppression” was performed by introducing TAG codons at positions 12, 65, and 357. A permissive aminoacyl synthetase/tRNA pair, pCNF, was used to incorporate O-methyl-L-tyrosine derivatives (Young '11). pEVOL-pCNF was transformed into BL21 DE3 cells alongside the biosensor plasmid. Cells were plated on double antibiotic selection plates (spectinomycin/ampicillin). A single colony was picked to inoculate a 5 mL primary LB culture, allowed to grow overnight at 37 C. This culture was used to inoculate a 200 mL autoinduction culture as described above. At OD600˜0.7, the unnatural amino acid was added to the culture dropwise while agitating. The culture was then allowed to incubate for a total of 30-32 h with shaking at 30 C.

Protein purification by FPLC: Biosensors were expressed in 200 mL autoinduction cultures and pelleted. The pellet was resuspended in 1×PBS, sonicated to lyse cells, and centrifuged. The resulting lysate contained the soluble biosensor and applied to a Ni-NTA column on a Akta FPLC. 1×PBS with 10 mM imidazole was used as the wash buffer and 1×PBS with 200 mM imidazole was used as the eluent. The biosensor was eluted with a linear gradient from 10 to 200 mM imidazole. 5 mL fractions were collected and analyzed by SDS-PAGE to confirm purity. Pure fractions were concentrated in a spin column with 30 kDa cutoff (Amicon). The protein was then buffer exchanged into 3×PBS pH 7.0 and concentrated to ˜500 uL. Absorbance measured at 280 nm and extinction coefficient calculated for aromatic residues were used to determine biosensor concentration.

Fluorescence Dose Response and S-Slope: Samples were added to flat black 96-well microtiter plates (Costar). All fluorescence measurements were conducted on a fluorescence plate reader using a “GFP program”: excitation centered at 485 nm (20 nm band; 30 flashes) and emission observed at 535 nm (25 nm band; 40 μs integration time). One set of biosensor responses is conducted with no ligand present to provide baseline fluorescence (F0). Each drug concentration-fluorescence response was conducted in triplicate. ΔF/F0 is computed at each [drug] point as (Fbiosensor+drug−F0)/F0. OriginPro9.1 was used to perform Hill curve fitting and compute Hill parameters. S-Slope is defined as Δ(F/F0)/(Δ[drug]) with units of μM-1 and provides a single metric of biosensor sensitivity (Bera '19). For Hill coefficient ˜1, S-Slope=(ΔFₘₐₓ/F0)/(EC50).

Directed evolution: site-saturation mutagenesis (SSM) was performed using either an “NNK” scheme were 32 codons are sampled to cover all amino acids or a “22-codon” scheme that minimizes bias in encoding each amino acid (Kille '13). SSM focused on residues within the binding pocket, second shell, and the two linkers between the PBP and cpGFP. In some embodiments, directed evolution consisted of (A) DNA library preparation, (B) culturing in 96-well plates, and (C) screening for response to ligands and obtaining winning sequences. (A) 22-codon method was used to create mutant DNA libraries. The PCR product library was transformed into TOP10 cells to amplify the DNA. Sequencing of mutants from each library was used to verify randomization. (B) 300 ng of the library was transformed into BL21 DE3 cells and plated on ampicillin selection plates and incubated overnight at 37° C. Autoduction media was prepared and 800 microliters were added to each well in a 96-deep well plate. A single colony was picked to inoculate each well. Aeraseal film was used to cover the plate while allowing oxygenation. The plate was incubated at 30 C with shaking at 250 rpm for 30 h. The culture was pelleted, resuspended in 3×PBS pH 7.0, frozen in liquid nitrogen, and thawed at room temperature to lyse bacteria. The plate was centrifuged again, providing biosensor solubilized in the lysate. (C) Lysate was transferred to a 96-well flat black plate. A Tecan plate reader was used to measure fluorescence in each well. 11 microliters of 10× drug solution was added to each well and mixed by shaking. The fluorescence was measured after ligand application. ΔF/F0 was computed for each well. The top ˜8 mutants were sequenced. Non-parent mutants were then transformed into BL21 and used to inoculate a 10 mL autoinduction culture. The lysate was then used for a full dose response to verify the advantageous mutation.

Opioid Panel x Biosensor Library Screen: The biosensor library was composed of selected proteins from previously completed evolution projects toward nicotine, acetylcholine, ketamine, and serotonin biosensors (Shivange '19, Bera '19, and Unger '19). 111 nM purified biosensor stock solutions were prepared in 3×PBS pH 7.0. A liquid handling robot (epMotion, Eppendorf) was used to mix 100 μL of a stock biosensor solution and 11 μL of 2 mM drug solution in a 96-well microtiter plate. Some wells in the drug plate contained each buffer used for opioid panel preparation in triplicate. In one negative control plate, 3×PBS pH 7.0 with no biosensor replaced the stock biosensor solution to determine background fluorescence from the compound alone. In all cases, fluorescence was read using the plate reader's GFP program. ΔF/F0 was computed for each biosensor x drug pair: (Fbiosensor+drug−Fdrug−F0)/F0. The results of this single point screen were visualized using a “green map”. A “hit” is defined for a drug-biosensor pair with ΔF/F0>0.5 at 200 μM drug.

Selectivity Screens: dDpamine, norepinephrine, and serotonin were prepared in 3×PBS pH 7.0 with 100 μM sodium ascorbate and used immediately for a single dose-response experiment. Acetylcholine, choline, and ATP dilution plates were prepared for each day's experiment. All other neurotransmitter and opioid solutions were prepared and stored at −20° C. when not in use. Fluorescence measurements were conducted as in “Fluorescence Dose Response”.

Isothermal Titration Calorimetry: Isothermal titration calorimetry was performed using an Affinity ITC (TA Instruments). All experiments were conducted in 3×PBS pH 7.0. The [biosensor] in the sample cell was prepared at 10-20× the Kd (approximated as the fluorescence EC50 for the first experiment). The [drug] in the syringe was prepared at 10× the set [biosensor]. Each experiment allowed equilibration to 25° C. for ˜20 minutes before the first drug injection. 2.0-2.5 μL injections of the drug solution (˜0.1 mol equivalent) were performed every 300 s for 20 iterations. The data was analyzed in NanoAnalyze (TA Instruments). The heat from the first injection was discarded. The resulting curve was fit using a combination of NanoAnalyze's “independent” and “constant” functions representing a single binding site and a constant heat from drug dissolution respectively. The resulting enthalpy, entropy, Kd, and stoichiometry are reported.

Stopped flow kinetics: Stopped flow kinetics was measured using an Applied Photophysics SX20 stopped flow fluorimeter with 490 nm LED excitation and 510 nm long pass filter at room temperature (22° C.). Equal volumes of 0.2 μM biosensor and varying concentrations of drug were mixed (5 replicates measured). The first 3 ms are not analyzed to discount mixing artifacts and instrument dead time. Data was plotted and time courses were fit, when possible, to a single rising exponential (y-intercept+total rise*(1−exp(−kobs*t))) using Kaleidagraph (version 4.4). kobs was plotted as a function of [ligand]. The linear portion of that graph was fit, with the slope reporting kl and the y-intercept reporting k−1.

Generation and analysis of racemic methadone steady-state concentration-response relation: The relaxation data were sampled at a rate of one sample per ms. The steady-state concentration-response relation for ΔF/F0 vs [racemic methadone] were measured by taking the mean ΔF/F0's for the final 10 ms of the 1 s methadone stopped-flow relaxations. ΔF was computed by subtracting the fluorescence in methadone from that in 0 μM methadone. After correcting for instrumental offset, the value of F0 was 0.05. The data were fit to the Hill equation without weighting using the nonlinear regression routine provided by the Origin 2018 software.

Global analysis of R- and S-methadone competition: To determine whether R- and S-methadone bound competitively to the sensor, the effect of fixed R-methadone concentrations (0, 0.1, 1, 10, 100 μM) on the S-methadone concentration-response relation was measured. R-methadone is not a simple competitive inhibitor because it also activates the sensor. Therefore, a model from enzyme kinetics for competitive inhibition with mixed alternative substrates was adapted to analyze the data (Segel, 1993). According to this model, the following equation (1) describes the S-methadone concentration-response relation in the presence of a fixed [R-methadone],

$$\frac{Δ F}{F 0} = \frac{F {max}_{S \frac{[ S ]}{{K}_{s}}} + F {max}_{R \frac{[ R ]}{{K}_{R}}}}{1 + \frac{[ S ]}{{K}_{S}} + \frac{[ R ]}{{K}_{R}}} , \qquad eq . 1$$

where the Fmax_{S} and Fmax_{R} are the maximum ΔF/F₀'s for S- and R-methadone alone, the [S] and [R] are the [S-methadone] and [R-methadone], and the K_{S} and K_{R} are the equilibrium dissociation constants for S- and R-methadone binding (in this case, EC50s for S- and R-methadone activation of the sensor). The data were fit to equation 1 using the global nonlinear regression routine provided by the Origin 2018 software. The Fmax_{S}, Fmax_{R}, K_{S}, and K_{R} were shared parameters. The value of F₀ for all the data was the sensor fluorescence in buffer alone. The parameters values obtained from the global fit represented the single set of values that fit all the data optimally.

Lyophilization and stability test: 100 μM biosensor stock solution in 3×PBS pH 7.0 was prepared and 100 μL was aliquoted into each of three microcentrifuge tubes. All aliquots were flash frozen using liquid nitrogen. One aliquot was stored in a −80° C. freezer. The other two aliquots were lyophilized overnight, yielding a yellow-green powder. These two samples were stored in an opaque container at room temperature and humidity for 3 weeks. Each aliquot was reconstituted with 100 μL deionized water to achieve the original biosensor and salt concentrations. All three aliquots were used to prepare 111 nM biosensor solutions in 3×PBS pH 7.0 for dose response with fentanyl.

Adeno associated virus preparation: biosensor (e.g., iS-methadoneSnFR) gene was cloned into a pAAV vector with synapsin promoter and a PDGFR plasma membrane-targeting sequence. Integrity of the inverted terminal repeat sequence was confirmed by SmaI digest. Mammalian tissue culture, virus harvesting, and virus purification were performed according to an established protocol (Challis 2019). HEK293T cells were transfected with the pAAV, pHelper, and PHP.eb capsid genes. The media was harvested after 3 and 5 days post-transfection and the cells were harvested at 5 days-post transfection. Digestion produced a lysate with soluble viral particles. The lysate was purified by an ultracentrifuge gradient column. Viral titer was determined by qPCR.

Tissue culture and transfection: HeLa cells (ATCC) were thawed and passaged twice before use in imaging studies. Cell culturing followed ATCC recommended protocols. For each imaging study, 100 k HeLa cells were plated onto a dish with a 10 mm coverlsip (MatTek) and incubated at 37 C, 5% CO2 for 24 h. Cells were then transfected with Lipofectamine 3000 using 500 ng for _PM, 500 ng for _cyto, 250 ng for _ER, and _600 ng for _Golgi constructs in OptiMEM. Cells were kept in OptiMEM transfection medium for 24 h and then switched to standard growth media for an additional 24 h before imaging.

Dose response in human sweat: Human sweat was collected by the Gao lab and stored at 4° C. The solution was turbid due to oils from skin. The aliquot was mixed by agitation and then 50 μL was added to each well of a microtiter plate. A 222 nM biosensor solution was prepared in 6×PBS pH 7.4 and 50 μL of this solution was mixed into the wells with sweat. 11 μL of a 10× drug serial dilution was added sequentially to each well and the contents of each well thoroughly mixed. Fluorescence was read on the Tecan Spark 10M using the GFP program.

Primary neuron culturing and transduction: A timed pregnant mouse was euthanized at embryonic day 16. The uterine sac was removed and each embryo was decapitated before dissection. The hippocampi from several embryos were combined and digested with 50 U of papain at 37 degrees C. for 15 min. After DNase treatment, the cells were triturated in HBSS with 5% donor equine serum, and spun through a layer of 4% BSA and HBSS. Dishes with a 10 mm poly-D-lysine-coated glass bottom (MatTek) were coated with poly-L-ornithine and laminin 24 h prior to plating. The cells were then plated at a density of 90,000/dish in 130 microliters of plating medium. After 1 h, 3 mL of complete culture medium was added to each dish. Half of the medium was changed twice a week. After 4 days, the neurons were transduced by mixing virus into the media. After ˜2 weeks, the dishes were used in imaging experiments.

Dose response in mouse serum: Cardiac puncture was performed on wildtype C57BL/6 mice. Mice were first anesthetized for 10 minutes using 5% isoflurane. A 23-gauge needle was used to puncture the heart and light backpressure was used to withdraw blood. Samples were centrifuged to separate sera. Dose responses were conducted using serum from two different male mice sacrificed on different days. Serum was diluted to 50% with a biosensor/drug solution in 6× PBS pH 7.4. The final biosensor concentration was 100 nM and the final solution was buffered by 3×PBS. Fluorescence was read on the Tecan Spark 10M using the GFP program.

Expression in cultured mammalian cells: Biosensor plasmids were transferred to pDisplay vectors (Invitrogen) with either plasma membrane or endoplasmic reticulum-targeting sequences (Shivange '19). HeLa cells (ATCC) were cultured according to ATCC's protocol. The cells were plated on 35-mm imaging dishes with built in 15 mm diameter glass coverslips (MatTek) such that the cells reached ˜70% confluency 48 hours later. Lipofectamine 3000 (ThermoFisher) was used according to manufacturer's protocol to transfect 250 ng of the ER-plasmid and 500 ng of the PM-plasmid in OptiMEM.

Time-resolved dose-response imaging in mammalian cells: An Olympus IX-81 outfitted with an automated solution changer was used to image the biosensors in live mammalian cells. A 40× (NA 1.0, oil) objective was used. A blue LED with peak emission at ˜470 nm (LZ1-10DB00, LED Engin) illuminated cells and imaging was performed ˜4 fps. Generally, cells with lower F0 were chosen for imaging and analysis. Elevated syringes with flow controlled by solenoid valves (Automate Scientific) were used to deliver solutions by gravity flow. Opioid solutions were prepared in HBSS pH 7.4 (Gibco) by serial dilution. The solution changer (ValveBank8, Automate Scientific) was programmed to delivered “ascending-descending” or “stuttered step” drug/wash pulses. In “ascending-descending” programs, solutions are applied from the highest to the lowest [opioid] and then lowest to highest; the analysis computes the average of the two response at each [opioid]. In “stuttered step” programs, the same [opioid] is applied and washed out twice before moving to the next, higher [opioid]; the second response in each set was used for final analysis.

Analysis of Cellular Imaging Time Series Data: ImageJ plugin “Time Series Analyzer” was used to calculate the average pixel intensity in the region of interest (ROI) drawn (PM, ER, or Golgi) and a background region in each frame. These data were then handled in OriginLabs software. The background values were subtracted from the ROI at each frame to calculate ‘F’. A baseline was drawn with a spline to determine ‘F0’ at each frame. ΔF/F0 was then calculated as (F−F0)/F0 for each frame. The steady-state response was considered as the average of the final 20 frames in each response. For the S-slope measurements, the HBSS response was subtracted from each of the responses at 50-250 nM drug. The final linear fit was constrained to y-intercept=0.

Acute slice preparation and imaging: adeno associated viruses with AAV9 capsid were generated (Challis 2019) where the biosensor gene is under a hSyn promoter and targeted to the cytoplasm (by omitting any targeting sequences). A titer of 3*10 vg was delivered to the ventral tegmental area (coordinates injected: M/L: −0.40, A/P: −3.00, D/V: −4.30) of C57BL/6 mice. 2 weeks were given for recovery from surgery and viral expression to stabilize. The animal was terminally anesthetized with ketamine and perfused. The brain was removed, glued to a sample block, and sliced using a compresstome for 300 micron coronal slices. 20 min recovery in a HEPES buffer was followed by 1 h recovering in artificial cerebrospinal fluid. Slices were imaged on an upright microscope with 4× (air) and 40× (water immersion) objectives under blue LED widefield illumination. Drug was perfused during imaging.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A polypeptide, comprising a first mutated periplasmic binding protein (PBP) domain and a second PBP domain connected to the first PBP domain, wherein at least one of the first and second PBP domain comprises at least one amino acid substitution mutation selected from the positions functionally equivalent to K10, N11, Q15, T43, T68, T325, K330, D341, Y357, A360, E391, R395, V405, F436, H455, and D490 of the amino acid sequence of SEQ ID NO: 29

2.-4. (canceled)

5. An opioid biosensor, comprising a first mutated periplasmic binding protein (PBP) domain and a second PBP domain connected to the first PBP domain, wherein the opioid biosensor is capable of undergoing a detectable conformational change upon binding to an opioid,
wherein at least one of the first and second PBP domain comprises at least one amino acid substitution mutation selected from the positions functionally equivalent to K10, N11, Q15, T43, T68, T325, K330, D341, Y357, A360, E391, R395, V405, F436, H455, and D490 of the amino acid sequence of SEQ ID NO: 29.

6. The opioid sensor of claim 5, the opioid is an opioid specific to a kappa opioid receptor, delta opioid receptor, or mu opioid receptor.

7. The opioid sensor of claim 5, the opioid is an opioid selected from the group consisting of codeine, morphine, morphinan, fentanyl, phenyl piperazine, hydrocodone, oxycodone, oxymorphone, heroin, and benzenoid monoamine.

8. The opioid biosensor of claim 5, wherein the first PBP domain comprises an amino acid sequence at least 80% identical to position 1-75 of a sequence selected from SEQ ID NOs: 1-27 and the second PBP domain comprises an amino acid sequence at least 80% identical to positions 325-521 of a sequence selected from SEQ ID NOs: 1-27.

9. The opioid biosensor of claim 5, wherein the first PBP domain comprises an amino acid sequence at least 80% identical to position 1-75 of SEQ ID NO: 29 and the second PBP domain comprises an amino acid sequence at least 80% identical to positions 325-521 of SEQ ID NO: 29.

10. The opioid biosensor of claim 5, wherein the first PBP domain comprises an amino acid sequence at least 90% identical to position 1-75 of a sequence selected from SEQ ID NOs: 1-27 and the second PBP domain comprises an amino acid sequence at least 90% identical to positions 325-521 of a sequence selected from SEQ ID NOs: 1-27.

11. The opioid biosensor of claim 5, wherein the first PBP domain comprises an amino acid sequence at least 90% identical to position 1-75 of SEQ ID NO: 29 and the second PBP domain comprises an amino acid sequence at least 90% identical to positions 325-521 of SEQ ID NO: 29.

12. The opioid biosensor of claim 5, wherein the first PBP domain comprises an amino acid sequence of position 1-75 of a sequence selected from SEQ ID NOs: 1-25 and the second PBP domain comprises an amino acid sequence of positions 325-521 of a sequence selected from SEQ ID NOs: 1-25.

13. The opioid biosensor of claim 5, wherein the at least one amino acid substitution mutation comprises a substitution mutation at F436.

14. opioid biosensor of claim 5, wherein the at least one amino acid substitution mutation comprises a substitution mutation at N11.

15. The opioid biosensor of claim 5, wherein the at least one amino acid substitution mutation comprises a substitution mutation of N11V or a substitution mutation homologous to N11V.

16. The opioid biosensor of claim 5, wherein the polypeptide or the opioid biosensor comprises a substitution mutation of N11V and a Phe at position 436 and the opioid biosensor binds specifically to S-methadone.

17.-19. (canceled)

20. The opioid biosensor of claim 5, wherein the at least one amino acid substitution mutation comprises a substitution mutation of N11E or a substitution mutation homologous to N11E.

21.-23. (canceled)

24. The opioid biosensor of claim 5, wherein the polypeptide or the opioid biosensor comprises a Gly at position 357.

25.-29. (canceled)

30. The opioid biosensor of claim 5, further comprising a reporter connected to the first PBP domain and the second PBP domain on either end.

31.-36. (canceled)

37. A nucleic acid encoding the opioid biosensor of claim 5.

38.-43. (canceled)

44. A device, comprising the opioid biosensor of claim 5.

45. (canceled)

46. (canceled)

47. A method of detecting an opioid in a sample, the method comprising:
providing an opioid biosensor of claim 5;
contacting the polypeptide, opioid biosensor or the device with a sample suspected of containing an opioid; and
detecting the conformational change that can be trigged by the binding of the opioid, thereby determining the presence or absence of the opioid in the sample.

48. The method of claim 47, wherein detecting the conformational change of the opioid biosensor comprises detecting a signal emitted by the opioid biosensor, and optionally the signal is emitted by the reporter of the opioid biosensor.

49.-61. (canceled)


SEQUENCE LISTING (35 sequences)

SEQ ID NO: 1 - protein, 521 aa, Artificial Sequence
  Synthetic
ANDTVVVGSANFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPSTDPEGAYETVKKEYKRKWNIVWLKPLGFNNTGTLTVKDELAKQYNLKTFSDLAKISDKLILGATMFFLEAPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 2 - protein, 521 aa, Artificial Sequence
  Synthetic
ANDTVVVGSANFTEGIIVANMVAEMIEAHTDLKVVRKLNLGGGNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPISTDPEGAYETVKKEYKRKWNIVWLKPLGFNNTGTLTVKDELAKQYNLKTFSDLAKISDKLILGATMFFLEAPDGYPGLQKLYNFKFKHTKSMDMGIRYTAIDNNEVQVIDAWATDGLLVSHKLKILEDDKAFFPPYYAAPIIRQDVLDKHPELKDVLNKLANQISGEEMQKLNYKVDGEGQDPAKVAKEFLKEKGLI

SEQ ID NO: 3 - protein, 521 aa, Artificial Sequence
  Synthetic
ANDTVVVGSIEFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAAATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 4 - protein, 521 aa, Artificial Sequence
  Synthetic
ANDTVVVGSIVFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAFATDGLLVSHKLKILEDDKAFFPPYYAAPIIRQDVLDKHPELKDVLNKLANQISAEEMQKLNYKVDGEGQDPAKVAKEFLKEKGLI

SEQ ID NO: 5 - protein, 521 aa, Artificial Sequence
  Synthetic
ANDTVVVGSIIFTETIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKWFFPPYYAAPIIRQDVLDKHPELKDVLNKLANQISLEEMQKLNYKVDGEGQDPAKVAKEFLKEKGLI

SEQ ID NO: 6 - protein, 521 aa, Artificial Sequence
  Synthetic
ANDTVVVGSIEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPMTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 7 - protein, 521 aa, Artificial Sequence
  Synthetic
ANDTVVVGSIEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPVTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 8 - protein, 521 aa, Artificial Sequence
  Synthetic
ANDTVVVGSIEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPRTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 9 - protein, 521 aa, Artificial Sequence
  Synthetic
ANDTVVVGSIEFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDATATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 10 - protein, 521 aa, Artificial Sequence
  Synthetic
ANDTVVVGSIEFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDACATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 11 - protein, 521 aa, Artificial Sequence
  Synthetic
ANDTVVVGSIEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 12 - protein, 521 aa, Artificial Sequence
  Synthetic
ANDTVVVGSIEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLERPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 13 - protein, 521 aa, Artificial Sequence
  Synthetic
ANDTVVVGSINFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPSTDPEGAYETVKKEYKRKWNIVWLKPLGFNNTGTLTVKDELAKQYNLKTFSDLAKISDKLILGATMFFLEAPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 14 - protein, 521 aa, Artificial Sequence
  Synthetic
ANDTVVVGSIPFTEGIIVANMVAEMIEAHTDLKVVRKLNLGGINVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKGFFPPYYAAPIIRQDVLDKHPELKDVLNKLANQISLEEMQKLNYKVDGEGQDPAKVAKEFLKEKGLI

SEQ ID NO: 15 - protein, 521 aa, Artificial Sequence
  Synthetic
ANDTVVVGSIPFTEGIIVANMVAEMIEAHTDLKVVRKLNLGGINVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKQFFPPYYAAPIIRQDVLDKHPELKDVLNKLANQISLEEMQKLNYKVDGEGQDPAKVAKEFLKEKGLI

SEQ ID NO: 16 - protein, 521 aa, Artificial Sequence
  Synthetic
ANDTVVVGSIFFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 17 - protein, 521 aa, Artificial Sequence
  Synthetic
ANDTVVVGSILFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 18 - protein, 521 aa, Artificial Sequence
  Synthetic
ANDTVVVGSIPFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 19 - protein, 521 aa, Artificial Sequence
  Synthetic
ANDTVVVGSIDFTEGIIVANMVAEMIEAHTDLKVVRKLNLGGVNVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPATTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 20 - protein, 521 aa, Artificial Sequence
  Synthetic
ANDTVVVGSVEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 21 - protein, 521 aa, Artificial Sequence
  Synthetic
ANDTVVVGSAEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 22 - protein, 521 aa, Artificial Sequence
  Synthetic
ANDTVVVGSMEFTEGIIVANMVAEMIEAHTDLKVVRKLNLGGVNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 23 - protein, 521 aa, Artificial Sequence
  Synthetic
ANDTVVVGSANFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNFAPPPSTDPEGAYETVKKEYKRKWNIVWLKPLGFNNTGTLTVKDELAKQYNLKTFSDLAKISDKLILGATMFFLEAPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 24 - protein, 521 aa, Artificial Sequence
  Synthetic
ANDTVVVGSIEFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNFEPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAAATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 25 - protein, 521 aa, Artificial Sequence
  Synthetic
ANDTVVVGSIVFTEGIIVANMVAEMIEAHTDLKVVRKLNLGGENVNFEAIKRGGANNGIDIYVEYTGHGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLSWGVQCFARYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAFATDGLLVSHKLKILEDDKAFFPPYYAAPIIRQDVLDKHPELKDVLNKLANQISAEEMQKLNYKVDGEGQDPAKVAKEFLKEKGLI

SEQ ID NO: 26 - protein, 522 aa, Artificial Sequence
  Synthetic
ANDTVVVGSINFTEQIIVANMVAEMIEAHTDLKVVRKLNLGGTNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPSTDPEGAYETVKKEYKRKWNIVWLKPLGFNNTYTLAVKDELAKQYNLKTFSDLAKISDKLILGATMFFLEKPDGYPGLQKLYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLIL

SEQ ID NO: 27 - protein, 521 aa, Artificial Sequence
  Synthetic
ANDTVVVGSINFTEGIIVANMVAEMIEAHTDLKVVRKLNLGGENVNFEAIKRGGANNGIDIYVEYTGHGLVDILGFPEPNVYITADKQKNGIKANFKIRHNMEDGSVQLADHYQQNTPIGDGPVLLPDNHYLSTQSVLSKDPNEKRDHMVLLEFVTAAGITLGMDELYKGGTGGSMSKGEELFTGVVPILVELDGGVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPSSTDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI

SEQ ID NO: 28 - protein, 272 aa, Artificial Sequence
  Synthetic
ANDTVVVGSKNFTEQIIVANMVAEMIEAHTDLKVVRKLNLGGTNVNFEAIKRGGANNGIDIYVEYTGTGLVDILGTTDPEKAYETVKKEYKDKWNIVWLKPLGFNNTYTLAVKDELAKQYNLKTFSDLAKISDKLILGATMEFLERPDGYPGLQKVYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLI

SEQ ID NO: 29 - protein, 521 aa, Artificial Sequence
  Synthetic
ANDTVVVGSKNFTEQIIVANMVAEMIEAHTDLKVVRKLNLGGTNVNFEAIKRGGANNGIDIYVEYTGTGLVDILGFPEPNVYITADKQKNGIKANFKIRHNVEDGSVQLADHYQQNTPIGDGPVLLPDNHYLSTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFPPPPTTDPEKAYETVKKEYKDKWNIVWLKPLGFNNTYTLAVKDELAKQYNLKTFSDLAKISDKLILGATMEFLERPDGYPGLQKVYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLI

SEQ ID NO: 30 - protein, 75 aa, Artificial Sequence
  Synthetic
ANDTVVVGSKNFTEQIIVANMVAEMIEAHTDLKVVRKLNLGGTNVNFEAIKRGGANNGIDIYVEYTGTGLVDILG

SEQ ID NO: 31 - protein, 197 aa, Artificial Sequence
  Synthetic
TTDPEKAYETVKKEYKDKWNIVWLKPLGFNNTYTLAVKDELAKQYNLKTFSDLAKISDKLILGATMEFLERPDGYPGLQKVYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLI

SEQ ID NO: 32 - protein, 75 aa, Artificial Sequence
  Synthetic
ANDTVVVGSINFTEQIIVANMVAEMIEAHTDLKVVRKLNLGGTNVNFEAIKRGGANNGIDIYVEYTGTGLVDILG

SEQ ID NO: 33 - protein, 198 aa, Artificial Sequence
  Synthetic
STDPEGAYETVKKEYKRKWNIVWLKPLGFNNTYTLAVKDELAKQYNLKTFSDLAKISDKLILGATMFFLEKPDGYPGLQKLYNFKFKHTKSMDMGIRYTAIDNNEVQVIDAFATDGLLVSHKLKILEDDKHFFPPYYAAPIIRQDVLDKHPELKDVLNKLANQISDEEMQKLNYKVDGEGQDPAKVAKEFLKEKGLIL

SEQ ID NO: 34 - protein, 75 aa, Artificial Sequence
  Synthetic
ANDTVVVGSINFTEGIIVANMVAEMIEAHTDLKVVRKLNLGGENVNFEAIKRGGANNGIDIYVEYTGHGLVDILG

SEQ ID NO: 35 - protein, 197 aa, Artificial Sequence
  Synthetic
STDPEGAYETVKKEYKRKWNIVWLKPLGFNNTYTLTVKDELAKQYNLKTFSDLAKISDKLILGATMFFLEGPDGYPGLQKLYNFKFKHTKSMDMGIRYTAIDNNEVQVIDAWATDGLLVSHKLKILEDDKAFFPPYYAAPIIRQDVLDKHPELKDVLNKLANQISLEEMQKLNYKVDGEGQDPAKVAKEFLKEKGLI